SEQUENCE LISTING (92 sequences)

SEQ ID NO: 1 - protein, 533 aa, Oryza sativa
MASSNRHWPSMFRSKHATQPWQTQPDMAGSPPSLLSGSSAGSAGGGGYSLKSSPFSSVGEERVPDPKPRWNPRPEQIRILEAIFNSGMVNPPRDEIPRIRMQLQEYGQVGDANVFYWFQNRKSRSKNKLRSGGTGRAGLGLGGNRASAPAAAHREAVAPSFTPPPPILPAPQPVQPQQQLVSPVAAPTSSSSSSSDRSSGSSKPARATSTQAMSVTTAMDLLSPLAAACHQQMLYQGQPLESPPAPAPKVHGIVPHDEPVFLQWPQSPCLSAVDLGAAILGGQYMHLPVPAPQPPSSPGAAGMFWGLCNDVQAPNNTGHKSCAWSAGLGQHWCGSADQLGLGKSSAASIATVSRPEEAHDVDATKHGLLQYGFGITTPQVHVDVTSSAAGVLPPVPSSPSPPNAAVTVASVAATASLTDFAASAISAGAVANNQFQGLADFGLVAGACSGAGAAAAAAAPEAGSSVAAVVCVSVAGAAPPLFYPAAHFNVRHYGDEAELLRYRGGSRTEPVPVDESGVTVEPLQQGAVYIVVM

SEQ ID NO: 2 - protein, 515 aa, Oryza sativa
MASPNRHWPSMFRSNLACNIQQQQQPDMNGNGSSSSSFLLSPPTAATTGNGKPSLLSSGCEEGTRNPEPKPRWNPRPEQIRILEGIFNSGMVNPPRDEIRRIRLQLQEYGQVGDANVFYWFQNRKSRTKNKLRAAGHHHHHGRAAALPRASAPPSTNIVLPSAAAAAPLTPPRRHLLAATSSSSSSSDRSSGSSKSVKPAAAALLTSAAIDLFSPAPAPTTQLPACQLYYHSHPTPLARDDQLITSPESSSLLLQWPASQYMPATELGGVLGSSSHTQTPAAITTHPSTISPSVLLGLCNEALGQHQQETMDDMMITCSNPSKVFDHHSMDDMSCTDAVSAVNRDDEKARLGLLHYGIGVTAAANPAPHHHHHHHHLASPVHDAVSAADASTAAMILPFTTTAAATPSNVVATSSALADQLQGLLDAGLLQGGAAPPPPSATVVAVSRDDETMCTKTTSYSFPATMHLNVKMFGEAAVLVRYSGEPVLVDDSGVTVEPLQQGATYYVLVSEEAVH

SEQ ID NO: 3 - protein, 280 aa, Oryza sativa
MTLLAVIVFGGGGGGSRSSALPSLSTGVAGVEGSATGPWLPESLTLNAATHRIPSTSPLSIPQTLTITRDPPYPMLPRSHGHRTGGGGFSLKSSPFSSVGEERVPDPKPRRNPRPEQIRILEAIFNSGMVNPPRDEIPRIRMQLQEYGQVGDANVFYWFQNRKSRSKNKLRSGGTGRAGLGLGGNRASEPPAAATAHREAVAPSFTPPPILPPQPVQPQQQLVSPVAAPTSLSSSSSDRSSGSSKPARATLTQAMSVTAAMDLLSPLRRSARPRQEQRHV

SEQ ID NO: 4 - protein, 514 aa, Zea mays
MASSFNNKTSHWPSMFRSKHAAEPWQAQPDISSSPPSLLSGGGSSSTTTIGRCLKHPLSGYSGGEERTPDPKPRWNPRPEQIRILEAIFNSGMVNPPRDEIPRIRMRLQEYGQVGDANVFYWFQNRKSRSKNKQRTGQLGLGLARAPGCGAAAPPVTPQPLIQNQFQVLASPAQAPASSSSSSSDRSSGSSKPAPQPMSATAAAMNFPGPLGAACAQMYYQAHPVAPVSALPAHKVQDPVASDEPVFQPWLQGYFLSAAEVASILGGQYRHDVPVQQQPPATLPAGAFLGLYNEVTEPTVTGHRTCAWGPAGLGQFWPVGGADHHQHHKHNTTAATNTVARDAAHEHATTLGLLQYGFEASAAMETASAAVPLAASPGTAASVATAGLTSLPASTNAVVVNYDLLQGLAVPGSGSGAVGVSTGGAPPVAVAAAPTAAQEGVVVALCITDSVTGKSVAHNVAAARLDVRAQFGEAAVLLRAVGDRGGLDLVPVPVDALGCTVEPLQHGAFYYVLV

SEQ ID NO: 5 - protein, 506 aa, Zea mays
MASSNRHWPSMYRSSLACNFQQPQPQPDMNNGGKSSLMSSRCEENGGRNPEPRPRWNPRPEQIRILEGIFNSGMVNPPRDEIRRIRLQLQEYGPVGDANVFYWFQNRKSRTKHKLRAAGQLQPSGSGRSALQARACAPAPVTPPRNLQLAAAAPVAPPTSSSSSSSDRSSGSSSSKSVTVTPTTAVALASPAGAAPAAVFRQQGVMPTTAMDLLTPLPSSSAALAARQLYYQYHSQIMAPAAPPMPDTVIASPEQFLPQWQQGGQQHYYLPATELGGVLDGHSHHTHEPPAAIHRPVSLSPSVLFGLCNEALRQDYCADISVVPTKGLGHGHQFWNSTTCGSDMGNSNSKIDAVSAVIRDDEKSRLGLLHYYGLAGATTTAAAAVAPAPLAADAAAGTATLLPSSAASDQLQGLLDAAGLLMGETPPTPTATVVAVARDAVTCAATATAQFSVPASMRLDVRLAFGEAALLARHTGEAVPVDESGVTVEPLQQDTLYYVLMQATNN

SEQ ID NO: 6 - protein, 515 aa, Zea mays
MASSSFNNSHWPSMFRSKHAAEPCQTTQPDISSSPPSLLSAGGASTTTTTGRCLKHSISVGGEERAPDPKPRWNPRPEQIRILEAIFNSGMVNPPRDEIPRIRMRLQQYGQVGDANVFYWFQNRKSRSKNKLRSSTAGTGRLGLQGLARAPGRGAAAAPPPVEPPPLVQNQFHMLASPAQAPTSSSSSSSDRSSGSSKPAAEPAMPATAAPMDLLGPLAAACPQMYYQGSPVAPAHKVLDLVASVEPVFQPWPQGYCLSAAEVATILGGQYMHVPVQQQPPAPLPAGALLGLCNDVTEPTAVVTGHKTCAWGPAGLGQSWPCGGADHHQPGKNNNTAARELAHEDDATKLGLLQYGFGATTAMEAAPAVAPLAASPAGGAVTMASVSASTAGLTGFPASTNGVVANYDLLQGLAVPGGGAGAGRAPAAVAVAADAAPTAAQEGVVALCITDSITGKSVAHNVAAARLDVRAQFGEAAVLLRCGGERGLDLEPVPVDASGCTVEPLQRGAFYYVLL

SEQ ID NO: 7 - DNA, 1602 bp, unidentified
  source 1..1602: mol_type = unassigned DNA
atggcgtcgtcgaacaggcactggccgagcatgttcaggtcgaagcacgccacgcagccgtggcagacgcagcctgacatggccgggtcgccgccctccctcctctccggctcctccgccggcagcgccggcggcggcggctactccctcaagtcgtcgcccttctcgtcagtgggcgaggagagggttccggacccgaagccgcggtggaacccgcggccggagcagatccggatcctggaggcgatcttcaactccggcatggtcaacccgccgcgcgacgagatcccgcgcatccgcatgcagctgcaggagtacggccaggtcggcgacgccaacgtcttctactggttccagaaccgcaagtcccgctccaagaacaagctgcgctccggcgggacaggccgcgcggggctcggcctcggcggcaaccgggcctccgcgccggcggcggcgcaccgggaggccgtggcgccgtcgttcacgccgccgccaccaatcctcccggcgccccagccggtgcagccgcagcagcagcttgtctcgcctgtggcggcgcctacctcgtcgtcgtcttcctcctccgaccgttcgtccgggtccagcaagcctgcgagggctacgtcgacgcaggcgatgtccgtgacgacggccatggacctgctctcgccgctcgccgcggcgtgccaccagcagatgctctatcaaggccagccactggagtcgccgccggcgcctgctcccaaagtgcacggcatcgtgccacacgacgagccggtcttcctgcagtggccgcagagcccctgcctgtcggccgtcgacctcggcgccgccattcttggcggccagtacatgcacctgccggtgcccgctccgcagccaccgtcgtcgccgggcgcggcgggcatgttctgggggctctgcaacgacgtgcaagcgccaaacaacaccggccacaagagctgcgcctggagcgccgggctcggccagcactggtgcggctccgccgatcagctcggcctcggcaagagcagcgcggcgtcgatcgccaccgtgtctaggccggaggaggcgcacgacgtcgacgccacgaagcacggtctgctacagtacggctttggcatcaccacgccgcaagtgcacgtggacgttacctcctcggctgctggcgttctgcctcctgttccgtcctcgccgtcgccgccgaacgccgccgtcaccgtcgcgagcgtggccgccaccgctagcctgactgattttgctgcaagtgctatatctgctggcgccgtcgctaacaatcagtttcaaggtctcgcggatttcgggctcgtcgccggcgcctgctccggcgccggagccgccgccgccgccgccgcgcccgaggcgggcagttccgtggccgcggttgtgtgcgtcagcgtcgcgggcgccgcgccgccgctcttctacccggccgcgcacttcaacgtgaggcactacggcgacgaggccgagctgctccgctacagaggaggcagccgcacggagcctgtgcccgtcgacgagtcgggcgtcaccgtcgagccgctccagcagggcgccgtctacattgttgtcatgtaa

SEQ ID NO: 8 - DNA, 1548 bp, unidentified
  source 1..1548: mol_type = unassigned DNA
atggcctcaccgaacaggcactggccgagcatgttcaggtccaatcttgcctgcaacatccagcagcagcagcagcctgacatgaacggcaacggcagctcgtcctcttccttcctcctctcgccacctactgctgcgaccaccggcaacggcaagccctccttgctctcctcagggtgtgaggaggggacgaggaatccggagccgaagccgcggtggaacccgaggccggagcagataaggatactggaggggatcttcaactccgggatggtgaacccgccgcgcgacgagatccgccgcatccgcctgcagctgcaggagtacggccaggtcggcgacgccaacgtcttctactggttccagaaccgcaagtcccgcaccaagaacaagctgcgcgccgccggccaccaccaccaccacggccgcgccgccgccctgccgcgcgcgtcggcgccgccgtcgacgaacatcgtactcccctctgcagcggcggcggcgcccttgacgccgccgcggcgccatctcctcgccgcgacctcctcctcgtcctcctcctccgaccgctcctccgggtccagcaagtcggtgaaaccagctgctgccgcgctgctgacgtcagccgccatcgaccttttctcgccggcgccggcgccgacgacccagctgcccgcgtgccagctctactaccatagccatcccacgccgctggcacgtgatgatcagctcatcacctcgccggagtcgtcgtcgctcctcctgcagtggccggcgagccagtacatgccggcgacggagctcggcggcgtcctcggctcgtcgtcccacacgcaaaccccggcagcgatcaccacccacccatcgacgatctcacccagcgtgctcctcggcctatgcaacgaggcactagggcagcatcagcaagagaccatggacgacatgatgatcacctgctccaacccctccaaggtgttcgaccaccattccatggacgacatgagctgcaccgacgcggtgagcgccgtgaacagggacgacgagaaggcgaggctggggttactgcactacggcatcggcgtcactgctgctgcaaatccggcaccacatcatcatcatcatcatcatcatcttgcctctcctgtgcatgatgctgtctcggctgcagatgctagtacggcggccatgatccttccattcaccaccactgctgctgcgacgccgagcaacgtcgtcgctacaagctctgcactcgctgatcagttgcaagggctgttggatgctgggttgctgcagggaggggcggcgccgccgccgccctcggcgacggtggtggcggtgagccgcgacgacgagacgatgtgcaccaagaccacgagctacagcttcccggcgacgatgcacctcaacgtgaagatgttcggcgaggcggccgtgctggtgcgctacagcggcgagccggtgctcgtcgacgactccggcgtcaccgtcgagccgctgcagcagggcgcgacctactacgtgctggtatctgaggaagctgtgcattga

SEQ ID NO: 9 - DNA, 921 bp, Oryza sativa
  source 1..921: mol_type = genomic DNA
atgatggccttaggcgtgccaccgcctccctcgcgcgcctacgtgtccggcccgctacgcgacgatgacacttttggcggtgatcgtgttcggcggcggcggcggtggctcaaggagcagtgccctgccatcattgtccacgggggtggcaggcgtggaggggtcggccacagggccctggctgccggagtctctaaaatgcgtctcccagccctaaacgccgccacccaccggatcccctccacctcgcccttaagtatccctcagaccctcaccatcacccgcgatcctccctacccaatgctgcctcgaagtcacggccaccggaccggcggcggcggcttctccctcaagtcctcgcccttctcgtcagtgggcgaggagagggttccggacccgaagccgcggcggaacccgcggccggagcagatccggatcctggaggccatcttcaactccggcatggtcaacccgccgcgcgacgagatcccgcgcatccgcatgcagctgcaggagtacggccaggtcggcgacgccaacgtcttctactggttccagaaccgcaagtcccgctccaagaacaagctgcgctccggcgggacaggccgcgcggggctcgggctcggcggcaaccgggcctcagagccgccggcggcggcgacggcgcaccgggaggccgtggcaccgtcgttcacgccgccaccgatcctcccgccccagccggtgcagccgcagcagcagcttgtctcgccggtggcggcgcccacctcgttgtcgtcatcgtcctccgaccgctcgtccgggtccagcaagcccgcgagggctacgttgacgcaggcgatgtccgtgacggcggccatggacctgctctcgccgctccgccgatcagctcggccacggcaagagcagcgccatgtctag

SEQ ID NO: 10 - protein, 559 aa, Oryza sativa
MASITNWLGFSSSSFSGAGADPVLPHPPLQEWGSAYEGGGTVAAAGGEETAAPKLEDFLGMQVQQETAAAAAGHGRGGSSSVVGLSMIKNWLRSQPPPAVVGGEDAMMALAVSTSASPPVDATVPACISPDGMGSKAADGGGAAEAAAAAAAQRMKAAMDTFGQRTSIYRGVTKHRWTGRYEAHLWDNSCRREGQTRKGRQVNAGGYDKEEKAARAYDLAALKYWGTTTTTNFPVSNYEKELDEMKHMNRQEFVASLRRKSSGFSRGASIYRGVTRHHQHGRWQARIGRVAGNKDLYLGTFGTQEEAAEAYDIAAIKFRGLNAVTNFDMSRYDVKSIIESSNLPIGTGTTRRLKDSSDHTDNVMDINVNTEPNNVVSSHFTNGVGNYGSQHYGYNGWSPISMQPIPSQYANGQPRAWLKQEQDSSVVTAAQNLHNLHHFSSLGYTHNFFQQSDVPDVTGFVDAPSRSSDSYSFRYNGTNGFHGLPGGISYAMPVATAVDQGQGIHGYGEDGVAGIDTTHDLYGSRNVYYLSEGSLLADVEKEGDYGQSVGGNSWVLPTP

SEQ ID NO: 11 - protein, 584 aa, Arabidopsis thaliana
MNSMNNWLGFSLSPHDQNHHRTDVDSSTTRTAVDVAGGYCFDLAAPSDESSAVQTSFLSPFGVTLEAFTRDNNSHSRDWDINGGACNNINNNEQNGPKLENFLGRTTTIYNTNETVVDGNGDCGGGDGGGGGSLGLSMIKTWLSNHSVANANHQDNGNGARGLSLSMNSSTSDSNNYNNNDDVVQEKTIVDVVETTPKKTIESFGQRTSIYRGVTRHRWTGRYEAHLWDNSCKREGQTRKGRQVYLGGYDKEEKAARAYDLAALKYWGTTTTTNFPLSEYEKEVEEMKHMTRQEYVASLRRKSSGFSRGASIYRGVTRHHQHGRWQARIGRVAGNKDLYLGTFGTQEEAAEAYDIAAIKFRGLSAVTNFDMNRYNVKAILESPSLPIGSSAKRLKDVNNPVPAMMISNNVSESANNVSGWQNTAFQHHQGMDLSLLQQQQERYVGYYNGGNLSTESTRVCFKQEEEQQHFLRNSPSHMTNVDHHSSTSDDSVTVCGNVVSYGGYQGFAIPVGTSVNYDPFTAAEIAYNARNHYYYAQHQQQQQIQQSPGGDFPVAISNNHSSNMYFHGEGGGEGAPTFSVWNDT

SEQ ID NO: 12 - protein, 579 aa, Brassica napus
MNNNWLGFSLSPYEQNHHRKDVYSSTTTTVVDVAGEYCYDPTAASDESSAIQTSFPSPFGVVVDAFTRDNNSHSRDWDINGCACNNIHNDEQDGPKLENFLGRTTTIYNTNENVGDGSGSGCYGGGDGGGGSLGLSMIKTWLRNQPVDNVDNQENGNAAKGLSLSMNSSTSCDNNNDSNNNVVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEVEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENSASGWQNAAVQHHQGVDLSLLHQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQQSPGGDFPAAMTNNVGSNMYYHGEGGGEVAPTFTVWNDN

SEQ ID NO: 13 - protein, 579 aa, Brassica napus
MNNNWLGFSLSPYEQNHHRKDVCSSTTTTAVDVAGEYCYDPTAASDESSAIQTSFPSPFGVVLDAFTRDNNSHSRDWDINGSACNNIHNDEQDGPKLENFLGRTTTIYNTNENVGDIDGSGCYGGGDGGGGSLGLSMIKTWLRNQPVDNVDNQENGNGAKGLSLSMNSSTSCDNNNYSSNNLVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEIEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENNASGWQNAAVQHHQGVDLSLLQQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQHSPGGDFPAAMTNNVGSNMYYHGEGGGEVAPTFTVWNDN

SEQ ID NO: 14 - protein, 559 aa, Oryza sativa
MASITNWLGFSSSSFSGAGADPVLPHPPLQEWGSAYEGGGTVAAAGGEETAAPKLEDFLGMQVQQETAAAAAGHGRGGSSSVVGLSMIKNWLRSQPPPAVVGGEDAMMALAVSTSASPPVDATVPACISPDGMGSKAADGGGAAEAAAAAAAQRMKAAMDTFGQRTSIYRGVTKHRWTGRYEAHLWDNSCRREGQTRKGRQVYLGGYDKEEKAARAYDLAALKYWGTTTTTNFPVSNYEKELDEMKHMNRQEFVASLRRKSSGFSRGASIYRGVTRHHQHGRWQARIGRVAGNKDLYLGTFGTQEEAAEAYDIAAIKFRGLNAVTNFDMSRYDVKSIIESSNLPIGTGTTRRLKDSSDHTDNVMDINVNTEPNNVVSSHFTNGVGNYGSQHYGYNGWSPISMQPIPSQYANGQPRAWLKQEQDSSVVTAAQNLHNLHHFSSLGYTHNFFQQSDVPDVTGFVDAPSRSSDSYSFRYNGTNGFHGLPGGISYAMPVATAVDQGQGIHGYGEDGVAGIDTTHDLYGSRNVYYLSEGSLLADVEKEGDYGQSVGGNSWVLPTP

SEQ ID NO: 15 - protein, 695 aa, Oryza sativa
MATMNNWLAFSLSPQDQLPPSQTNSTLISAAATTTTAGDSSTGDVCFNIPQDWSMRGSELSALVAEPKLEDFLGGISFSEQQHHHGGKGGVIPSSAAACYASSGSSVGYLYPPPSSSSLQFADSVMVATSSPVVAHDGVSGGGMVSAAAAAAASGNGGIGLSMIKNWLRSQPAPQPAQALSLSMNMAGTTTAQGGGAMALLAGAGERGRTTPASESLSTSAHGATTATMAGGRKEINEEGSGSAGAVVAVGSESGGSGAVVEAGAAAAAARKSVDTFGQRTSIYRGVTRHRWTGRYEAHLWDNSCRREGQTRKGRQVYLGGYDKEEKAARAYDLAALKYWGPTTTTNFPVNNYEKELEEMKHMTRQEFVASLRRKSSGFSRGASIYRGVTRHHQHGRWQARIGRVAGNKDLYLGTFSTQEEAAEAYDIAAIKFRGLNAVTNFDMSRYDVKSILDSAALPVGTAAKRLKDAEAAAAYDVGRIASHLGGDGAYAAHYGHHHHSAAAAWPTIAFQAAAAPPPHAAGLYHPYAQPLRGWCKQEQDHAVIAAAHSLQDLHHLNLGAAAAAHDFFSQAMQQQHGLGSIDNASLEHSTGSNSVVYNGDNGGGGGGYIMAPMSAVSATATAVASSHDHGGDGGKQVQMGYDSYLVGADAYGGGGAGRMPSWAMTPASAPAATSSSDMTGVCHGAQLFSVWNDT

SEQ ID NO: 16 - protein, 679 aa, Zea mays
MASANNWLGFSLSGQDNPQPNQDSSPAAGIDISGASDFYGLPTQQGSDGHLGVPGLRDDHASYGIMEAYNRVPQETQDWNMRGLDYNGGGSELSMLVGSSGGGGGNGKRAVEDSEPKLEDFLGGNSFVSDQDQSGGYLFSGVPIASSANSNSGSNTMELSMIKTWLRNNQVAQPQPPAPHQPQPEEMSTDASGSSFGCSDSMGRNSMVAAGGSSQSLALSMSTGSHLPMVVPSGAASGAASESTSSENKRASGAMDSPGSAVEAVPRKSIDTFGQRTSIYRGVTRHRWTGRYEAHLWDNSCRREGQSRKGRQVYLGGYDKEDKAARAYDLAALKYWGTTTTTNFPISNYEKELEEMKHMTRQEYIAYLRRNSSGFSRGASKYRGVTRHHQHGRWQARIGRVAGNKDLYLGTFSTEEEAAEAYDIAAIKFRGLNAVTNFDMSRYDVKSILESSTLPVGGAARRLKDAVDHVEAGATIWRADMDGAVISQLAEAGMGGYASYGHHGWPTIAFQQPSPLSVHYPYGQPSRGWCKPEQDAAAAAAHSLQDLQQLHLGSAAHNFFQASSSSTVYNGGAGASGGYQGLGGGSSFLMPSSTVVAAADQGHSSTANQGSTCSYGDDHQEGKLIGYDAAMVATAAGGDPYAAARNGYQFSQGSGSTVSIARANGYANNWSSPFNNGMG

SEQ ID NO: 17 - protein, 707 aa, Glycine max
MGSMNLLGFSLSPHEEHPSSQDHSQTTPSRFSFNPDGSISSTDVAGGCFDLTSDSTPHLLNLPSYGIYEAFHRNNSINTTQDWKENYNSQNLLLGTSCNKQNMNQNQQQQPKLENFLGGHSFGEHEQTYGGNSASTDYMFPAQPVSAGGGGSGGGSNNNNNSNSIGLSMIKTWLRNQPPNSENINNNNESGGNIRSSVQQTLSLSMSTGSQSSTSLPLLTASVDNGESPSDNKQPNTSAALDSTQTGAIETAPRKSIDTFGQRTSIYRGVTRHRWTGRYEAHLWDNSCRREGQTRKGRQVYLGGYDKEEKAARAYDLAALKYWGTTTTTNFPISHYEKELEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKSILESTTLPIGGAAKRLKDMEQVELSVDNGHRADQVDHSIIMSSHLTQGINNNYAGGGTATHHNWHNAHAFHQPQPCTTMHYPYGQRINWCKQEQQDNSDAPHSLSYSDIHQLQLGNNGTHNFFHTNSGLHPMLSMDSASIDNSSSSNSVVYDGYGGGGGYNVMPMGTTTAVVASDGDQNPRSNHGFGDNEIKALGYESVYGSATDSYHAHARNLYYLTQQQSSSVDTVKASAYDQGSACNTWVPTAIPTHAPRSTTSMALCHGATTPFSLLHE

SEQ ID NO: 18 - protein, 649 aa, Capsicum annuum
MKMKSMNDDSSSSNNSNSNNNNHSSAATNSNNWLGFSLSPHMKMEVTNASETQQQQHPHQQQFAQSFYLSSSPTTMNVSTASALCYENNPFHSSLSVMPLKSDGSLCIMEALSRSHADAMVQSSSPKLEDFLGGASQYGSHEREAMALSLDSLYYHQNDEDIQVHSHHPYYSPMHCHGMYQESLLEETKPTQISNCDAQMTGNEMKSWGHYAIDQHINDTCSMVAAAAAVAAGGGGGTVGCNDLQSLSLSMNPGTQSSCVTPRQISPTGLECVAIESKKRASAKVAQKQPVHRKSIDTFGQRTSQYRGVTRHRWTGRYEAHLWDNSCKKEGQTRKGRQVYLGGYDMEDKAARAYDQAALKYWGPSTHINFPLENYQKELEEMKNMTRQEYVAHLRRKSSGFSRGASIYRGVTRHHQHGRWQARIGRVAGNKDLYLGTFSTQEEAAEAYDVAAIKFRGVNAVTNFDISRYDVEKIMASNNLPAGELARRTKEREPRESIEYNNISVHKNEECVQNNNNNGNITDWKMVLYQASNPSIGSNNYRNPSFSVALQDLIGIDSINNSTSHATILDHEQNKIGANHFSNASSLVTSLGSSREASPDKTAASLVFAKPTKFVVPTTNVNACIPSAQLRPIPVSMAHLPVFAALNDA

SEQ ID NO: 19 - protein, 689 aa, Medicago truncatula
MASMNLLGFSLSPQEQHPSTQDQTVASRFGFNPNEISGSDVQGDHCYDLSSHTTPHHSLNLSHPFSIYEAFHTNNNIHTTQDWKENYNNQNLLLGTSCMNQNVNNNNQQAQPKLENFLGGHSFTDHQEYGGSNSYSSLHLPPHQPEASCGGGDGSTSNNNSIGLSMIKTWLRNQPPPPENNNNNNNESGARVQTLSLSMSTGSQSSSSVPLLNANVMSGEISSSENKQPPTTAVVLDSNQTSVVESAVPRKSVDTFGQRTSIYRGVTRHRWTGRYEAHLWDNSCRREGQTRKGRQVYLGGYDKEEKAARAYDLAALKYWGTTTTTNFPISHYEKEVEEMKHMTRQEYVASLRRKSSGFSRGASIYRGVTRHHQHGRWQARIGRVAGNKDLYLGTFSTQEEAAEAYDVAAIKFRGLSAVTNFDMSRYDVKTILESSTLPIGGAAKRLKDMEQVELNHVNVDISHRTEQDHSIINNTSHLTEQAIYAATNASNWHALSFQHQQPHHHYNANNMQLQNYPYGTQTQKLWCKQEQDSDDHSTYTTATDIHQLQLGNNNNNTHNFFGLQNIMSMDSASMDNSSGSNSVVYGGGDHGGYGGNGGYMIPMAIANDGNQNPRSNNNFGESEIKGFGYENVFGTTTDPYHAQAARNLYYQPQQLSVDQGSNWVPTAIPTLAPRTTNVSLCPPFTLLHE

SEQ ID NO: 20 - protein, 545 aa, Cenchrus ciliaris
MGSTNNWLRFVSFSGGGGAKDAAALLPLPPSPRGDVDEAGAEPKLEDFLGLQEPSAAAVGAGRPFAVGGGASSIGLSMIKNWLRSQPAPAGPAAGVDSMVLAAAAASTEVAGDGAEGGGAVADAVQQRKAAAVDTFGQRTSIYRGVTKHRWTGRYEAHLWDNSCRREGQTRKGRQVYLGGYDKEEKAARAYDLAALKYRGTTTTTNFPMSNYEKELEEMKHMSRQEYVASLRRKSSGFSRGASIYRGVTRHHQHGRWQARIGSVAGNKDLYLGTFSTQEEAAEAYDIAAIKFRGLNAVTNFDMSRYDVKSIIESSSLPVGGAPKRLKEVPDQSDMGININGDSAGHMTAINLLTDGNDSYGAESYGYSGWCPTAMTPIPFQFSIGHDHSRLWCKPEQDNAVVAALHNLHHLQHLPAPVGTHNFFQPSPVQDMTGVADASSPPVESNSFLYNGDVGYHGAMGGSYAMPVATLVEGNSAGSGYGVEEGTGSEIFGGRNLYSLSQGSSGANTGKADAYESWDPSMLVISQKSANVTVCHGAPVFSVWK

SEQ ID NO: 21 - protein, 542 aa, unidentified
  source 1..542: note = Pennisetum squamulatum
MGSTNNWLRFASFSGGGGAKDAAALLPLPPSPRGDVDEAGAEPKLEDFLGLQEPSAAAVGAGRPFAVGGGASSIGLSMIRNWLRSQPAPAGPAAGVDSMVLAAAAASTEVAGDGAEGGGAVADAVQQRKAAAVDTFGQRTSIYRGVTKHRWTGRYEAHLWDNSCRREGQTRKGRQGGYDKEEKAARAYDLAALKYRGTTTTTNFPMSNYEKELEEMKHMSRQEYVASLRRKSSGFSRGASIYRGVTRHHQHGRWQARIGSVAGNKDLYLGTFSTQEEAAEAYDIAAIKFRGLNAVTNFDMSRYDVKSIIESSSLPVGGTPKRLKEVPDQSDMGININGDSAGHMTAINLLTDGNDSYGAESYGYSGWCPTAMTPIPFQFSNGHDHSRLWCKPEQDNAVVAALHNLHHLQHLPAPVGTHNFFQPSPVQDMTGVADASSPPVESNSFLYNGDVGYHGAMGGSYAMPVATLVEGNSAGSGYGVEEGTGSEIFGGRNLYSLSQGSSGANTGKADAYESWDPSMLVISQKSANVTVCHGAPVFSVWK

SEQ ID NO: 22 - protein, 545 aa, unidentified
  source 1..545: note = Pennisetum squamulatum
MGSTNNWLRFASFSGGGGAKDAAALLPLPPSPRGDVDEAGAEPKLEDFLGLQEPSAAAVGAGRPFAVGGGASSIGLSMIRNWLRSQPAPAGPAAGVDSMVLAAAAASTEVAGDGAEGGGAVADAVQQRKAAAVDTFGQRTSIYRGVTKHRWTGRYEAHLWDNSCRREGQTRKGRQVYLGGYDKEEKAARAYDLAALKYRGTTTTTNFPMSNYEKELEEMKHMSRQEYVASLRRKSSGFSRGASIYRGVTRHHQHGRWQARIGSVAGNKDLYLGTFSTQEEAAEAYDIAAIKFRGLNAVTNFDMSRYDVKSIIESSSLPVGGTPKRLKEVPDQSDMGININGDSAGHMTAINLLTDGNDSYGAESYGYSGWCPTAMTPIPFQFSNGHDHSRLWCKPEQDNAVVAALHNLHHLQHLPAPVGTHNFFQPSPVQDMTGVADASSPPVESNSFLYNGDVGYHGAMGGSYAMPVATLVEGNSAGSGYGVEEGTGSEIFGGRNLYSLSQGSSGANTGKADAYESWDPSMLVISQKSANVTVCHGAPVFSVWK

SEQ ID NO: 23 - protein, 832 aa, Rosa canina
MNMNWLGFSLSPQEDDHAPISHQLADQETLASRLGFNSNEIQGAGGGTDVSGGGSSECFDVTNSDSTASVNHHLTPSIFGIHEAAAFNRNNDHIHSQDWNMKGAGMNSSDSNNYKRTSSSDLSTMLMGSNTSTTSTSYSIISQQANLENHHQQLPKLENFLGRHSFADHDSSSAGHDYMFDMNNGPGPVSSNVMNTKTNSNNIGLSMIKTWLRNQPSQPRDNHHQLEQESKNSKESRNQPQSSLSLSMGTGSLQTVTTATAGGAATGETTSSSDKNTKQSPVVTATTTTGTDAQTQSTGGAIEAVPRKAIDTFGQRTSIYRGVTRHRWTGRYEAHLWDNSCRREGQTRKGRQVYLGGYDKEDKAARAYDLAALKYWGTTTTTNFPISSYEKEIDEMKPMTRQEYVASLRRKSSGFSRGASIYRGVTRHHQHGRWQARIGRVAGNKDLYLGTFSTQEEAAEAYDIAAIKFRGLNAVTNFDMSRYDVKSILESSALPITTGATAKRLKDVQQQQPPPPADHHHQIMLSSVLDHHGQVIRSSSSTEHDIMSNVYSAYGSYGAQQGCSWPTLAFNQAQAQAAAAPHQAPFAAGINGMQLHYSPYGYGYGNAHAQRVWCKQEQDTNSNQERSFHHQDDDHLRQQLQLGGTHNFFHDHDQQQQQQQQQTSGLMGLMDSSAASMEHSSGSNSVIYSGGDHHGNNNGYGSSTTGTGGGYIMPMVMSTVVANDDQNQADGNNNNINGFGDGDDQEANIKAQQLGYDHHPQNMFLGSSSTIDPAYQHHASNRNLYYHLPVQDDQHESSSVAVATSSTTCNMNWVPTAVPTLAHPTFTVWNDT

SEQ ID NO: 24 - protein, 828 aa, Rosa canina
MNMNWLGFSLSPQEDDHAPISHQLADQETLASRLGFNSNEIHGAGGGTDVSGGGSSECFDLTNSDSTASVNHHLTPSIFGIHEAAAFNRNNDHIHSQDWNMKGAGMNSSDSNNYKRTSSSDLSTMLMGSNTTTSTSCSIISQQANLENHHQQLPKLENFLGRHSFADHDRSSAGHDYMFDMNNGPGPVSSNVMNTKTNSNNIGLSMIKTWLRNQPSQPRDNHHQLEQESKNSKESRNQPQSSLSLSMGTGSLQTVTTATAGGAATAATGETTCSSDKNTKQSPVVTATTTTGTDAQTQSTGGAIEAVPRKAIDTFGQRTSIYRGVTRHRWTGRYEAHLWDNSCRREGQTRKGRQVYLGGYDKEDKAARAYDLAALKYWGTTTTTNFPISSYEKEIDEMKPMTRQEYVASLRRKSSGFSRGASIYRGVTRHHQHGRWQARIGRVAGNKDLYLGTFSTQEEAAEAYDIAAIKFRGLNAVTNFDMSRYDVKSILESSALPITTGATAKRLKEVQQQQPPPPADHHHQIMLSSVLDHHGQIIRSSSSTEHDIMSNVYSAYGSYGAQQGCSWPTLAFNQAQAQAAAAPHQAPFAAGINGMQLHYSPYGYGYGNAHAQRVWCKQEQDTNSNQESSFHHQDDDHLRQQLQLGGTHNFFHDHDQQQTSGLMGLMDSSAASMEHSSGSNSVIYSGGDHHGNNNGYGSSTTGTGGGYIMPMVMSTVVANDDQNQADGNNNNINGFEDGDDQEANIKAQQLGYDHHHQNMFLGSSSTIDPAYQHHASNRNLYYHLPVQDDQHESSSVAVATSSTTCNMNWVPTAVPTLAHPTFTVWNDT

SEQ ID NO: 25 - protein, 549 aa, Ricinus communis
  VARIANT 302: note = Any amino acid
MAPATTNWLSFSLSPMEMLRSSTESQFISYEGSSTATPSPHYFIDNFYANGWGNPKEAQGATTMAAETSILTSFIDPETHHQQVPKLEDFLGDSSSIVRYSDNSQTDTQDSSLTHIYDQGSAAYFSEQQDLKAIAGFQAFSTNSGSEVDDSASIARTHLGGEFMGHSIDSSGNDQLGGFSNCTAANNALSLAVNNNNNNNGNQSATNSKTIAPVIESDCPKKIADTFGQRTSIYRGVTRHRWTGRYEAHLWDNSCRREGQARKGRQGALFFLFSPSSSYHLSLFVACFFNYSSVKILGIYAXSAIPHGHVLYFFQVTNYTKELDEMKYVSKQEFIASLRRKSSGFSRGASIYRGVTRHHQQGRWQARIGRVAGNKDLYLGTFATEEEAAEAYDIAAIKFRGMNAVTNFEMSRYDVEAIMKSALPIGGAAKRLKLSLESEQKPNLNHEQQPQGSSSNSSSNNISFASMPPVTAIPCGIPFENTTAQLYHHHHHHHHHQHHNLFHHLQTTNNNLGGTTDISSGSTTSSMATTMSMLPQTAEFFLWPHHQSY

SEQ ID NO: 26 - protein, 478 aa, Elaeis guineensis
MDMDTSHSWLAFSLSYHQPYLLEALSSAPPHGGGGMTAEERGGSAEVAAMAVVGPKLEDFLGGCGEPMGRYAGGETGDAGGIYDSELKHIAAGYLQGLPATEQQDSEMAKVAAPAESRKAVETFGQRTSIYRGVTRHRWTGRYEAHLWDNSCRREGQSRKGRQVYLGGYDKEEKAARAYDLAALKYWGPTTTTNFPISNYEKELEEMKNMTRQEFVASLRRKSSGFSRGASIYRGVTRHHQHGRWQARIGRVAGNKDLYLGTFSTQEEAAEAYDIAAIKFRGLNAVTNFDISRYDVKSIANSNLPIGGMTGRPSKATESSPSSSSDAMTVEAKQLLDGRDPSASLGFAALPIKHDQDFWSLFALQQQQQQQQQQSNQASGFGLFSSGVTMDFSTASNGVISQGCGGSLVWNGGVVGQQQEQSQNNSCSSIPYATPIAFGGNYEGSSYVGSWVTPPPSYYHEPAKPNVAVFQTPIFGME

SEQ ID NO: 27 - protein, 719 aa, Populus trichocarpa
MASMNNWLGFSLSHQELPSSQSDHHQDHSQNTDSRLGFHSDEISGTNVSGECFDLTSDSTAPSLNLPATFGILEAFRNNQPQDWNMKSLGMNPDTNYKTASGLPIFMGTSCNSQTIDQNQEPKLENFLGGHSFGNHEHKLNGCNTMYDTTGDYVFQNCSLQLPSEATSNERTSNNGGGDNKNSSIGLSMIKTWLRNQPAPTQQDTNNKNNGGAQSLSLSMSTGSQSAASALPLLAVNGGVNNTGGDQSSSDNNKQQKSTTPSLDSQTGAIESVPRKSIDTFGQRTSIYRGVTRHRWTGRYEAHLWDNSCRREGQTRKGRQVYLGGYDKEEKAARAYDLAALKYWGTTTTTNFPITNYEKEIEEMKHMTRQEYVASLRRKSSGFSRGASIYRGVTRHHQHGRWQARIGRVAGNKDLYLGTFSTQEEAAEAYDIAAIKFRGLNAVTNFDMSRYDVNSILESSTLPIGGAAKRLKEAEHAEIAMDIAQRTDDHDNMGSQLTDGISSYGAVQHGWPTVAFQQAQPFSMHYPYGQRLWCKQEQDSDNRSFQELHQLQLGNTHNFFQPSVLHNLVSMDSSSMEHSSGSNSVVYSSGVNDGTSTGTNGGYQGIGYGSSAGYAVPMATVISNNDNNHNQGNGYGDGDQVKALGYENMFSPSDPYHARNLHYLSQQPSAGGIKASAYDQGSACYNWVPTAVPTIAAARSNNMAVCHGAQPFTVWNDGT

SEQ ID NO: 28 - protein, 718 aa, Populus trichocarpa
MASTNNWLGFSLSPQELPSSQSDHHDHPQNTDSRLRFHSDEISGTDVSGESFDLTSDSTAPSLNLPASFGILEAFRNNQSQDWNNMKRSGINEDTSYNTTSDVPIFMGSSCNSQNIDQNQEPKLENFLGGHSFGNHEHKLNVCSTMYGSTGHYMFHNCSLQLPSEDASNERTSSNGGADTSINNNNTNSSIGLSMIKTWLKNQPAPTQQDTNNKSNGGAQSLSLSMSTGSQSGSDLPLLAVNGGGNRTRGEQSSSDNNKQQKTTPSLDSQTGAIEVVPRKSIDTFGQRTSIYRGVTRHRWTGRYEAHLWDNSCRREGQTRKGRQGGYDKEDKAARAYDLAALKYWGTTTTTNFPMSNYEKEIEEMKHMTRQEHVASLRRKSSGFSRGASIYRGVTRHHQHGRWQARIGRVAGNKDLYLGTFSTQEEAAEAYDIAAIKFRGLNAVTNFDMNRYDVNSIMESSTLPIGGAAKRLKEAEHAEITTRVQRTDDHDSTSSQLTDGISNYGTAAHHGWPTIAFQQAQAFTMHYPYGQRLWCKQEQDSDNHSFQELHQLQLGNTQNFLQPSVLHNVMSMESSSMEHSSGSDSVMYSSGGHDGTGTGTNGSYQGIGYGSNTGYAIPMATVIANDVNTQDQGNGYGDGEVKALGYENMFSSSDPYHARNLYYLSQQSSAGVIKASAYDQGSTCNNWLPTAVPTIAARSNNMAVCHGAPTFTVWNEST

SEQ ID NO: 29 - protein, 789 aa, Larix gmelinii
MGSTSNWLAFSLSPHLTVDMPDSTQPRSTSAASNHSRHHNDFSNGTVHDCYELHPTDTMQMPLRPDGSLCILEALDRTQNNQDWQLKSLENPGSMDLESDVQSQQMMKSELSILAGGSSEQMSASIGRHKNVDQEGPKLEDFLGGASLRGHYNDARTDSIYGNDDAFDEKMMAPGLRDVVPNCLNGFDVTDTELSSGSKKTDQNQDSTRNINSIQNSLVQDSYDQNSNDQYMFQDCSLQLPPNSGANNMIGLSMIKTWLRSQPCPENKMNAATNSSTPTSAKDQSLGNLTNIQSLSLSMSPGSQSSSPLALPVQYQNTNADSPSSESKKRSLEKQSLVSVEATPRKSIDTFGQRTSIYRGVTRHRWTGRYEAHLWDNSCRREGQTRKGRQVYLGGYDKEEKAARAYDLAALKYWGPTTTTNFPTGNYEKELEEMKHMTRQEYVASLRRKSSGFSRGASIYRGVTRHHQHGRWQARIGRVAGNKDLYLGTFSSQEEAAEAYDIAAIKFRGLNAVTNFDMTRYDVNSILESSTLPIGGAAAKRIKDAEPSDPSVDGRRTDDEISSTISSQIADTLTSYGNAAYPNGHAGWPIIAFQQQTNPHAPAFYSQQRAAAGWCKQEHNNIQNHDLQLHFQSSTQNFLQPSMMTSNANTVLHNLMNLESSAQLDGTNTNSNSGLFSNISGNLAGNSLQMANSPIPSGITVCDSARTPFSTENDGSSTKNSSYNDNMLSNSDPFARGLYYLSQHSPSVVKANYENAAYNNWMTPAVQTLAPRPNLTVCHAPIFTVWNDT

SEQ ID NO: 30 - DNA, 1361 bp, Arabidopsis sp.
  source 1..1361: mol_type = genomic DNA
cctctttgtcaccgtcactcttctcctcgttctcaacgtctccagcagagcactcccgcccgtggcggattccaccaacatagcggctagactaaccggaggaggactgatgcagtgttgggatgcactctacgagctgaagtcatgtactaatgagatcgttctcttctttctcaacggtgagaccaaactcggctacggttgctgcaacgccgttgatgtcattaccactgattgttggccggcgatgcttacttctcttggctttacactggaggaaaccaatgtcctccgtggtttctgtcaatctccgaactccggcggttcttctccagctctttcccctgtcaaactttgataaatgttcctcgctgacgtaagaagacattagtaatggttataatatatagctttctatgaatgtatggtgagaaaatgtctgttcactgattttgagtttggaataaaagcatttgcgtttggtttatcattgcgtttatacaaggacagagatccactgagctggaatagcttaaaaccattatcagaacaaaataaaccattttttgttaagaatcagagcatagtaaacaacagaaacaacctaagagaggtaacttgtccaagaagatagctaattatatctattttataaaagttatcatagtttgtaagtcacaaaagatgcaaataacagagaaactaggagacttgagaatatacattcttgtatatttgtattcgagattgtgaaaatttgaccataagtttaaattcttaaaaagatatatctgatctagatgatggttatagactgtaattttaccacatgtttaatgatggatagtgacacacatgacacatcgacaacactatagcatcttatttagattacaacatgaaatttttctgtaatacatgtctttgtacataatttaaaagtaattcctaagaaatatatttatacaaggagtttaaagaaaacatagcataaagttcaatgagtagtaaaaaccatatacagtatatagcataaagttcaatgagtttattacaaaagcattggttcactttctgtaacacgacgttaaaccttcgtctccaataggagcgctactgattcaacatgccaatatatactaaatacgtttctacagtcaaatgctttaacgtttcatgattaagtgactatttaccgtcaatcctttcccattcctcccactaatccaactttttaattactcttaaatcaccactaagcttcgaatccatccaaaaccacaatataaaaacagaactctcgtaactcaatcatcgcaaaacaaaacaaaacaaaacaaaaaccccaaaaagaaagaata

SEQ ID NO: 31 - DNA, 678 bp, Oryza sativa
  source 1..678: mol_type = genomic DNA
atggaatgatggatgaatgttcacgttcttgagttcctaaatggtactaattttgcaaaaactttctatatgtgttttttgttaagaatgttgttttaaacccatcttttcactttataatatttaattaaatcgttcgtaccctcgaatagttattgcaaattatacttaactattcagtcattcagcacaaaagaacagggccatgaaattgtaatactagtacatttctgttcttttcttttctttttgaggttgtctgaaacacctgtatcttaaactatcgcagactagccaatgagtcgtactcacctgaaactgaaaccaagtgattaaccaagctggttcgacagtaattccatccataatgcagctccggagcccttcatatcctgcatgttactcaaacaacatccccacctcctcatttcctctcccctattgcattgcataattgcagaagattaagccgctaatgcataattacacattatttgtgtccactaattttccctttcccacacgctacgaaactcaaaagccggcctcctcgcctccttccctgaacgttactaatcgcgtcatgtataaatacagagcttgcccacgcaccggcacattgcatcgcactacgcacatctacacgatacccaagcagcaaagctagaaagaaaaacc

SEQ ID NO: 32 - DNA, 465 bp, Arabidopsis thaliana
  source 1..465: mol_type = genomic DNA
gttgccttatgatttcttcggtttcaagatgatcaaatagttatagatttcatgctcacacatgctcattagatgtgtacatactttacttacccaaatctattttctcgcaaagattttgatggtaaagctgatttggttctattgaactaaatcaaacgagtttcagactgagtgattctaatccggcccattagcccctaaacagacccactaattacgcagcttttaatagagtaattacacctagtttacccactaaaccactaagcactaattatctcacaatctaatgagcttccctcgtaattacttgggctttcactctaccatttatttgtaacagtcaagtctctactgtctctatataaactctctaaagttaacacacaattctcatcacaaacaaatcaaccaaagcaacttctactctttcttctttcgaccttatcaatctgttgagaa

SEQ ID NO: 33 - protein, 67 aa, Oryza sp.
PDPKPRWNPRPEQIRILEAIFNSGMVNPPRDEIPRIRMQLQEYGQVGDANVFYWFQNRKSRSKNKLR

SEQ ID NO: 34 - DNA, 705 bp, Oryza sativa
  source 1..705: mol_type = genomic DNA
atgcctgaagtgagaaattccggcggtagggcggcgctcgccgacccctcgggtggtgggttctttatcaggaggacgacgtcgccgccgggagccgtggcggtcaagccgctggctcggcgggccctgccgccgacgagcaacaaggagaacgtgccgccgtcctgggctgtgaccgtgagggctacacccaagaggaggagccccctgcccgagtggtacccgaggagcccactccgcgacatcacgtcagtcgtcaaggcagttgagaggaaaagtcgcctcggaaatgctgcggttcggcagcagatccagttgagtgaagattcttcacgatctgtggatccagcaactccagtacaaaaagaagaaggtgtccctcaaagcacaccaacaccaccaactcaaaaggccctggatgctgctgccccttgtcctggctcaacccaagctgttgcaagcacatcaacagcttacttggccgagggcaagccgaaggcatcatcttcttctccatctgactgctcctttcagacaccatccagaccaaatgatccagctcttgctgatctcatggagaaggaactgtccagctccatagagcagatagagaagatggtaaggaagaacctcaagagagctccgaaggctgctcagccttccaaggtgaccatccagaagcgcaccctgttgtccatgagatga

SEQ ID NO: 35 - protein, 234 aa, Oryza sativa
MPEVRNSGGRAALADPSGGGFFIRRTTSPPGAVAVKPLARRALPPTSNKENVPPSWAVTVRATPKRRSPLPEWYPRSPLRDITSVVKAVERKSRLGNAAVRQQIQLSEDSSRSVDPATPVQKEEGVPQSTPTPPTQKALDAAAPCPGSTQAVASTSTAYLAEGKPKASSSSPSDCSFQTPSRPNDPALADLMEKELSSSIEQIEKMVRKNLKRAPKAAQPSKVTIQKRTLLSMR

SEQ ID NO: 36 - DNA, 732 bp, Arabidopsis thaliana
  source 1..732: mol_type = genomic DNA
atgccagaagcaagagatcgaaccgagaggcctgtggattactcgactatatttgccaaccgacggagacatggtattttacttgacgagccagattcacggcttagtttgattgaatctccggtgaatccagatattgggtctattggtggaacgggcgggcttgtgagaggcaatttcactacatggaggcctggtaatggcagaggtggtcacactccatttagattgccacagggaagagagaatatgcccatagtgaccgctaggcgtggaagaggtggtggtttgttgccttcttggtatccaagaacacctctacgcgacataactcatattgtgcgggctattgagagaagaagaggagctgggactggaggagacgatggccgagttattgagatcccaactcatcgacaagttggtgttcttgaatctccagtaccactgtcaggagaacacaaatgctcgatggtcactcctggaccatctgtgggattcaagcgtagttgcccaccatcaactgctaaagttcaaaagatgttacttgacatcactaaagagatagctgaggaagaagctggcttcatcacacccgagaagaagctactcaattctattgacaaagttgagaaaattgtgatggcggagatccagaagttgaagagcactcctcaagctaaaagggaagagcgggagaagagggtgcggactttaatgactatgcgatga

SEQ ID NO: 37 - protein, 243 aa, Arabidopsis thaliana
MPEARDRTERPVDYSTIFANRRRHGILLDEPDSRLSLIESPVNPDIGSIGGTGGLVRGNFTTWRPGNGRGGHTPFRLPQGRENMPIVTARRGRGGGLLPSWYPRTPLRDITHIVRAIERRRGAGTGGDDGRVIEIPTHRQVGVLESPVPLSGEHKCSMVTPGPSVGFKRSCPPSTAKVQKMLLDITKEIAEEEAGFITPEKKLLNSIDKVEKIVMAEIQKLKSTPQAKREEREKRVRTLMTMR

SEQ ID NO: 38 - DNA, 1479 bp, Oryza sativa
  source 1..1479: mol_type = genomic DNA
atgaagcttaagatgaacaaggcctgcgacatcgcctccatctccgtcctccctccccggaggaccggagggagcagcggcgcgtcggcttccggttccgtggcggtggcggtggcgtctcagccgcggtcgcagccgctctcgcagtcgcagcagtccttctcgcagggcgcctccgcctcgctcttgcactcgcagtcgcagttctcgcaggtctccctcgacgacaacctcctcaccctcctcccttcccccacccgcgatcagagatttggcttgcatgatgactcatccaagaggatgtcctctttaccagccagttcagcttcttgcgcgcgagaagagtctcagctgcaactggcaaaattaccaagcaacccagtgcaccgctggaacccctccattgcagatactagatcaggtcaggttactaatgaggatgttgagcgcaaatttcagcatctggcaagctcagtacataagatggggatggtggtagactcagtccaaagtgacgtaatgcagttaaacagagccatgaaggaggcatcattagattctggtagcatacggcaaaagattgctgtccttgaaagctcacttcagcaaattcttaagggacaagacgatctcaaagcactctttggaagcagcacaaaacacaatcctgatcagacaagtgttctgaattctctaggcagcaaattgaatgagatatcctcgacccttgcaaccttgcagacacaaatgcaagcaagacaactgcagggtgatcagacaactgttctgaattctaatgccagcaaatcgaatgagatatcctcgactcttgcaaccctgcagacacaaatgcaagcagatataagacaactgcggtgtgacgtcttcagagtttttacaaaagagatggagggggttgttagagctatcaggtctgtcaatagtaggcctgctgcaatgcaaatgatggcagaccagagttaccaagtaccagtttcaaatggatggacccagattaaccagacaccagtagcagctggaaggtctccaatgaaccgagcaccagtagcagctggaaggtcccggatgaaccaattacctgaaacaaaagtgctttctgcacatttggtttatcctgcaaaggtgacagatctgaagccaaaggtggagcagggaaaggtaaaagcagctccacaaaagccgtttgcttcgagctactacagggtggcacctaaacaggaagaggtagcgattagaaaggtcaatatacaagtgccagcaaagaaggcaccagtcagcataatcatcgagtcggatgatgacagtgaaggacgtgcgtcctgcgtgattttgaagacagaaacaggtagcaaggagtggaaagtgacaaagcaaggcaccgaagagggcctggagatcctgcggagggcgaggaagaggaggaggagagagatgcagtccatcgtgctcgcatcctag

SEQ ID NO: 39 - protein, 492 aa, Oryza sativa
MKLKMNKACDIASISVLPPRRTGGSSGASASGSVAVAVASQPRSQPLSQSQQSFSQGASASLLHSQSQFSQVSLDDNLLTLLPSPTRDQRFGLHDDSSKRMSSLPASSASCAREESQLQLAKLPSNPVHRWNPSIADTRSGQVTNEDVERKFQHLASSVHKMGMVVDSVQSDVMQLNRAMKEASLDSGSIRQKIAVLESSLQQILKGQDDLKALFGSSTKHNPDQTSVLNSLGSKLNEISSTLATLQTQMQARQLQGDQTTVLNSNASKSNEISSTLATLQTQMQADIRQLRCDVFRVFTKEMEGVVRAIRSVNSRPAAMQMMADQSYQVPVSNGWTQINQTPVAAGRSPMNRAPVAAGRSRMNQLPETKVLSAHLVYPAKVTDLKPKVEQGKVKAAPQKPFASSYYRVAPKQEEVAIRKVNIQVPAKKAPVSIIIESDDDSEGRASCVILKTETGSKEWKVTKQGTEEGLEILRRARKRRRREMQSIVLAS

SEQ ID NO: 40 - DNA, 1152 bp, Arabidopsis thaliana
  source 1..1152: mol_type = genomic DNA
atggaggaaagttcaggactatcatcgatgaagttcttctccgatcaacacctctcttacgctgacattcttcttcctcacgaagctcgtgctagaatcgaagtttctgttctcaatctcctccgaattttgaactccccagatccagctatctccgatctctctctgatcaataggaagagaagcaatagttgtataaacaaagggatactcacagatgtttcatatatattcctctctacttcgtttactaagagttcattgacgaatgctaaaacagctaaagcttttgttcgtgtgtggaaagttatggaaatatgctttcagattctgcttcaagagaaacgagtcacacaaagagagctcttctataagttgctttgtgattcacctgattacttttcatctcagattgaagttaacagaagtgtccaagatgtggtagcacttctacgttgtagtagatacagtcttggtattatggcttcaagcagaggccttgttgctggaaggttatttctacaagaaccaggtaaggaagctgtagattgttcggcctgtggttcttcaggttttgctataactggagacttgaatttgctagacaataccatcatgagaactgatgctcgttatatcattattgtggaaaagcatgcgatctttcatcggctcgtggaagatcgtgtgttcaatcacattccttgcgtgttcatcacagcgaaagggtatccggatattgccacaaggtttttcctccaccggatgagcacaacttttcctgatctgccaattcttgttctagttgattggaatccagctgggttagctatactatgcaccttcaagtttggaagcatagggatgggacttgaagcatacagatacgcttgcaatgtgaagtggattggtctccgaggagatgatctgaatctgataccagaagagtctttggttcccttaaagccaaaagattcacagattgctaagagcttattgtcctccaaaatattgcaggaaaactacatagaggagttgtcactgatggttcaaactggtaaaagagcggaaattgaagctctctattgtcatggttataattatctcggtaaatatatagctaccaagatcgtgcaaggcaaatacatataa

SEQ ID NO: 41 - protein, 383 aa, Arabidopsis thaliana
MEESSGLSSMKFFSDQHLSYADILLPHEARARIEVSVLNLLRILNSPDPAISDLSLINRKRSNSCINKGILTDVSYIFLSTSFTKSSLTNAKTAKAFVRVWKVMEICFQILLQEKRVTQRELFYKLLCDSPDYFSSQIEVNRSVQDVVALLRCSRYSLGIMASSRGLVAGRLFLQEPGKEAVDCSACGSSGFAITGDLNLLDNTIMRTDARYIIIVEKHAIFHRLVEDRVFNHIPCVFITAKGYPDIATRFFLHRMSTTFPDLPILVLVDWNPAGLAILCTFKFGSIGMGLEAYRYACNVKWIGLRGDDLNLIPEESLVPLKPKDSQIAKSLLSSKILQENYIEELSLMVQTGKRAEIEALYCHGYNYLGKYIATKIVQGKYI

SEQ ID NO: 42 - DNA, 1827 bp, Oryza sativa
  source 1..1827: mol_type = genomic DNA
atgttctactcgcaccagctcctcgcgcggaaggctccgctcggccagatatggatggcggcgacgcttcactcgaagatcaaccggaagcggcttgacaagctcgacatcatcaaaatctgtgaggagattttgaacccgtcggtacccatggcactaaggctctccggaattctcatgggtggtgtggcgatcgtgtacgagaggaaggtgaaggctctgtatgatgatgtgtctcggtttctgattgagatcaacgaggcatggcgggtcaagccagtcgcagaccccaccgtacttcccaagggcaaaacccaagccaagtatgaagcagtaacactgccagagaatatcatggatatggatgtggagcagcccatgcttttctcagaggctgatactacaaggttccggggaatgcgtttggaggatttggatgaccaatacattaatgtcaacctagacgatgatgacttctcgcgcgctgagaatcatcaccaagctgatgcagaaaatatcaccctggctgataatttcgggtctgggcttggagagactgatgtgttcaatcgttttgagagattcgacataacagatgatgatgcaactttcaatgtcactcctgatggacacccacaggttccaagtaatctggttccttctccacctaggcaggaagactctcctcagcaacaagaaaaccatcatgctgcctcatcccctcttcacgaagaagctcaacaagggggggcatctgtaaaaaatgagcaagagcagcagaagatgaagggtcagcaacctgctaaatcatcaaagagaaaaaaacgtaggaaagatgatgaggtgatgatggataacgaccagataatgatcccaggaaatgtatatcaaacatggctgaaggatccatcaagcctcattaccaaaaggcacagaatcaacagtaaagttaatcttattcggtcaatcaagataagagacctcatggacttgcccctcgtttctctaatatcttccttggagaagtcacccttagaattttattatcctaaggaacttatgcagctttggaaggaatgtactgaagtcaagtccccaaaagctccatcttcaggagggcagcagtcatcatcaccagaacaacagcaaagaaacttgcctcctcaggcatttccaacccagcctcaggttgataatgacagggaaatgggatttcacccagtggactttgcagatgacatcgaaaaactccgaggaaacactagtggggaatatggaagagattatgatgcttttcacagtgatcatagtgttactcctggaagtcctgggctaagtcgcaggtctgcttcaagctctggtggctctggacggggatttacgcagttggatccagaagtacagttgccatccggaaggtccaagaggcagcattcatctggaaaaagctttgggaacctcgatccagttgaagaagaattcccattcgagcaagaacttagagatttcaagatgagaaggctttcagatgttgggccaactccagacctgctggaagaaatcgaacctactcaaaccccatatgaaaagaaatccaatcctatcgaccaggtcacacaatcaatccactcgtacctcaagctacactttgacaccccaggggcctcacagtctgaatcattaagtcagctagcacatgggatgactacagcaaaggctgcccgactcttctatcaagcatgcgttttagcaactcatgattttatcaaggttaaccagctggaaccatacggagacatcttgatctcgaggggaccaaagatgtga

SEQ ID NO: 43 - protein, 608 aa, Oryza sativa
MFYSHQLLARKAPLGQIWMAATLHSKINRKRLDKLDIIKICEEILNPSVPMALRLSGILMGGVAIVYERKVKALYDDVSRFLIEINEAWRVKPVADPTVLPKGKTQAKYEAVTLPENIMDMDVEQPMLFSEADTTRFRGMRLEDLDDQYINVNLDDDDFSRAENHHQADAENITLADNFGSGLGETDVFNRFERFDITDDDATFNVTPDGHPQVPSNLVPSPPRQEDSPQQQENHHAASSPLHEEAQQGGASVKNEQEQQKMKGQQPAKSSKRKKRRKDDEVMMDNDQIMIPGNVYQTWLKDPSSLITKRHRINSKVNLIRSIKIRDLMDLPLVSLISSLEKSPLEFYYPKELMQLWKECTEVKSPKAPSSGGQQSSSPEQQQRNLPPQAFPTQPQVDNDREMGFHPVDFADDIEKLRGNTSGEYGRDYDAFHSDHSVTPGSPGLSRRSASSSGGSGRGFTQLDPEVQLPSGRSKRQHSSGKSFGNLDPVEEEFPFEQELRDFKMRRLSDVGPTPDLLEEIEPTQTPYEKKSNPIDQVTQSIHSYLKLHFDTPGASQSESLSQLAHGMTTAKAARLFYQACVLATHDFIKVNQLEPYGDILISRGPKM

SEQ ID NO: 44 - DNA, 1884 bp, Arabidopsis thaliana
  source 1..1884: mol_type = genomic DNA
atgttgagactggagagtttgatagtaacagtgtggggaccagcgacgcttctagctcgaaaagctccgttgggtcagatatggatggccgctacattgcacgcgaagatcaaccggaagaaactagataagctcgatatcattcaaatctgcgaagagattttgaatccgtcggttccgatggctcttagactctccgggattcttatgggtggtgttgtgattgtttatgagaggaaagtgaagctcctattcgatgatgttaatcgctttctggttgaaattaatggagcttggcgcacaaaatctgttccggatcccactttactacctaaaggaaaaacccatgccaggaaagaggctgttacattgcctgagaacgaagaagctgattttggagattttgaacagactcgtaatgttcctaaatttggcaattacatggattttcagcagacttttatttccatgcggttagatgaatcccatgttaacaataaccccgagccagaagatcttggacagcagttccatcaagctgatgccgagaatatcacactctttgagtatcatggttcattccagaccaacaatgaaacatatgatcgttttgaaagatttgacatcgaaggagatgatgaaacacagatgaactccaatccaagagaaggcgctgaaatacctacaactctcatcccatcaccacctcgtcatcatgacattcccgaaggagtcaaccccacaagccctcagcgccaggagcaacaggagaatcgtagggacggatttgctgagcagatggaggaacaaaacataccggacaaagaggaacacgatagaccacaaccagcgaaaaagagagcaagaaagacagctacttcagcgatggattatgagcaaactattatcgctggtcatgtttaccagtcatggctccaggatacttctgacattctctgtaggggggaaaagagaaaggttcgaggaactatccggccagacatggaaagtttcaaacgtgcgaatatgccacctacacaactctttgaaaaggacagttcttacccgcctcagctttaccagctttggtcaaagaatactcaagttcttcaaacctcatcatctgaatctcgacatcctgatctccgtgcggaacaatctccagggtttgttcaggagagaatgcataaccaccatcaaacagaccatcatgagcgcagtgacacaagctcccaaaatcttgatagtcccgcagaaatactccggacagttcgtactgggaaaggtgcttcagtagaaagcatgatggctggatctcgagcaagccctgaaactattaaccgccaggctgctgatattaatgtcacgccattctattctggagatgatgtgagatccatgcctagtacaccatccgcacgtggagcagcttcaattaacaacatagagatcagctctaaaagtcgcatgcccaatagaaaaagaccaaattcctcaccaagaagaggactcgaaccagtggcggaagagagaccgtgggagcaccgtgaatatgagtttgagttttcaatgttacctgaaaaacgcttcacagccgataaagaaatactatttgaaactgcatctacacagactcaaaagccagtgtgcaatcaatcagacgagatgataacagatagcatcaaaagtcacctgaagacacactttgaaacacctggagctcctcaagtggaatctcttaacaagctcgctgttggaatggacagaaacgctgcagctaaactcttcttccaatcctgtgtgttagctactcgcggagtcatcaaggtaaaccaagcagagccttatggggacattctcattgcaagaggacccaacatgtaa

SEQ ID NO: 45 - protein, 627 aa, Arabidopsis thaliana
MLRLESLIVTVWGPATLLARKAPLGQIWMAATLHAKINRKKLDKLDIIQICEEILNPSVPMALRLSGILMGGVVIVYERKVKLLFDDVNRFLVEINGAWRTKSVPDPTLLPKGKTHARKEAVTLPENEEADFGDFEQTRNVPKFGNYMDFQQTFISMRLDESHVNNNPEPEDLGQQFHQADAENITLFEYHGSFQTNNETYDRFERFDIEGDDETQMNSNPREGAEIPTTLIPSPPRHHDIPEGVNPTSPQRQEQQENRRDGFAEQMEEQNIPDKEEHDRPQPAKKRARKTATSAMDYEQTIIAGHVYQSWLQDTSDILCRGEKRKVRGTIRPDMESFKRANMPPTQLFEKDSSYPPQLYQLWSKNTQVLQTSSSESRHPDLRAEQSPGFVQERMHNHHQTDHHERSDTSSQNLDSPAEILRTVRTGKGASVESMMAGSRASPETINRQAADINVTPFYSGDDVRSMPSTPSARGAASINNIEISSKSRMPNRKRPNSSPRRGLEPVAEERPWEHREYEFEFSMLPEKRFTADKEILFETASTQTQKPVCNQSDEMITDSIKSHLKTHFETPGAPQVESLNKLAVGMDRNAAAKLFFQSCVLATRGVIKVNQAEPYGDILIARGPNM

SEQ ID NO: 46 - DNA, 1329 bp, Arabidopsis thaliana
  source 1..1329: mol_type = genomic DNA
atgtcttcttcgtcgagaaatctatctcaggagaatccgattcctcgtccgaacttagccaagactcgaacctcactccgcgatgttggaaaccgtcgtgctcccctcggcgacatcacaaatcagaagaatggatctagaaatccttcaccgtcgtctactctggtgaattgttcaaataagatcggccaatctaagaaagcaccaaaacctgctttatctcgtaattggaatttgggaattctcgattccggtttacctcccaagccaaatgcgaaatcaaacataatcgttccttacgaagacaccgaattgctccaaagcgatgatagtcttctatgttcttcacctgcattatccttggatgcctctcctactcaatctgacccgtcaatttccactcatgactctttgacgaaccacgttgtagattacatggtcgagagcactactgatgatggaaatgatgatgatgatgatgagattgttaacattgatagtgacttgatggatccacagctttgtgcttcttttgcttgtgatatctacgagcatttgcgtgtatctgaggtgaacaaaagaccggctctagattacatggaaagaactcagtcaagcatcaatgctagcatgcgttctatactgattgactggcttgtggaggttgctgaagagtataggctttcgcccgagacgttgtatttggcagtaaactacgttgatcggtatcttacaggaaatgcaatcaacaagcaaaatctgcagctacttggtgttacctgcatgatgatagcagcaaaatatgaagaagtgtgtgtgccgcaagtggaggatttctgttacatcactgataacacatacttaagaaatgagcttttggagatggagtcttctgttctgaactacttgaagttcgaattaacaactccaacagcaaaatgtttcttgaggcgctttcttcgtgctgctcaaggcagaaaggaggtaccatcactgctgtctgagtgtctggcctgctatctcaccgaattatcgctgttagattacgctatgcttcgatacgctccatcacttgttgcagcctctgcagttttcttggcacaatacactctacacccttcaagaaaaccatggaatgctacgctagagcattacacatcgtacagggctaaacatatggaagcatgcgttaagaatcttcttcagctgtgtaatgagaaactctcatctgatgtggttgcaatcagaaagaagtacagtcaacacaaatacaagtttgcagcaaagaagctttgtcccacgtcactaccgcaagagcttttcctctga

SEQ ID NO: 47 - protein, 442 aa, Arabidopsis thaliana
MSSSSRNLSQENPIPRPNLAKTRTSLRDVGNRRAPLGDITNQKNGSRNPSPSSTLVNCSNKIGQSKKAPKPALSRNWNLGILDSGLPPKPNAKSNIIVPYEDTELLQSDDSLLCSSPALSLDASPTQSDPSISTHDSLTNHVVDYMVESTTDDGNDDDDDEIVNIDSDLMDPQLCASFACDIYEHLRVSEVNKRPALDYMERTQSSINASMRSILIDWLVEVAEEYRLSPETLYLAVNYVDRYLTGNAINKQNLQLLGVTCMMIAAKYEEVCVPQVEDFCYITDNTYLRNELLEMESSVLNYLKFELTTPTAKCFLRRFLRAAQGRKEVPSLLSECLACYLTELSLLDYAMLRYAPSLVAASAVFLAQYTLHPSRKPWNATLEHYTSYRAKHMEACVKNLLQLCNEKLSSDVVAIRKKYSQHKYKFAAKKLCPTSLPQELFL

SEQ ID NO: 48 - DNA, 1038 bp, Oryza sativa
  source 1..1038: mol_type = genomic DNA
atgtcgacgtgcgactcaatgaaaagcccagactttgagtatattgataatggggattcctcctcagttctaggttccttgcagcgaagagcaaacgagaacctgcgtatctcagaggatagagatgttgaagaaactaagtggaagaaggatgctccttccccaatggaaatcgaccaaatttgtgatgttgacaataactacgaggatccgcagttgtgtgctactcttgcttctgatatctacatgcacttgcgcgaggccgagaccaggaaacatccatcaaccgattttatggaaacactccaaaaggatgtaaacccaagcatgagagcgatcctgatagactggcttgtggaagtcgctgaagaatatcgtcttgttcctgatacattatacctgacagttaactacattgaccgttatctttctggcaatgagatcaatcgtcaaagactgcaattacttggagttgcttgtatgcttattgctgcaaaatacaaggagatatgtgcacctcaagtagaagaattctgctatataactgacaacacatacttcagagatgaggttttggaaatggaagcttctgtcctgaattacctgaagtttgaaatgactgcacctacagcaaaatgctttttgaggagatttgtccgtgttgcacaagtatctgatgaggatccagcattgcatcttgagttcctagccaattatgttgctgagctatcactgctggagtacaatctactttcttaccctccttcactagtagcggcatcagctattttcctggccaaattcatactgcagccagcaaagcacccttggaattccacccttgctcactacacacaatacaagtcgtcagagttaagcgactgcgttaaggcattgcaccgccttttctgtgttggtcctgggagtaaccttcctgcaatcagggagaagtatacccaacataagtacaaatttgtggcgaagaagccctgcccaccctcaataccgaccgaattctttcgcgactcaacatgctga

SEQ ID NO: 49 - protein, 345 aa, Oryza sativa
MSTCDSMKSPDFEYIDNGDSSSVLGSLQRRANENLRISEDRDVEETKWKKDAPSPMEIDQICDVDNNYEDPQLCATLASDIYMHLREAETRKHPSTDFMETLQKDVNPSMRAILIDWLVEVAEEYRLVPDTLYLTVNYIDRYLSGNEINRQRLQLLGVACMLIAAKYKEICAPQVEEFCYITDNTYFRDEVLEMEASVLNYLKFEMTAPTAKCFLRRFVRVAQVSDEDPALHLEFLANYVAELSLLEYNLLSYPPSLVAASAIFLAKFILQPAKHPWNSTLAHYTQYKSSELSDCVKALHRLFCVGPGSNLPAIREKYTQHKYKFVAKKPCPPSIPTEFFRDSTC

SEQ ID NO: 50 - DNA, 1071 bp, Oryza sativa
  source 1..1071: mol_type = genomic DNA
atgtctaaggaagatgctatgtcaactggtgattcaacggaaagccttgatattgattgccttgatgatggggactccgaagtggtatcttccttgcaacatttggcagatgataagcttcatatttctgacaacagggatgttgcaggtgtggcatccaaatggacgaagcatggttgtaattcagtagaaattgattatatcgtcgacattgacaacaaccatgaggatccacagctgtgtgcaactcttgcttttgacatttacaagcacttgcgagtggctgagaccaagaaaaggccttcaacagattttgtggaaaccattcagaagaacattgacacaagcatgagggcagtgttaatagactggcttgtggaagtcacagaagaatatcggcttgtacctgaaaccttatacctcacagtcaattacattgaccggtatctctcgagcaaggtgatcaatcggcggaaaatgcaattacttggtgtcgcttgcctgcttatagcttctaagtatgaagagatatgcccaccccaagtagaagagctctgctatatttctgacaatacatacactaaggatgaggttttgaaaatggaagcttctgtcctgaaatacttgaagtttgagatgactgcacctacaacaaaatgctttttgaggagatttctacgagctgctcaagtatgccatgaggctccagttttgcatcttgagttcctagctaattacattgcggagctatcacttctggagtacagcttaatttgctatgtaccgtcacttatagctgcgtcttctattttcttggcgaagtttatccttaagccaacagagaatccttggaattcaacactttcattctacacacaatacaaaccatccgacctatgcaattgtgcaaaaggactacaccggcttttcttggttggccctggaggcaaccttcgagcagttagagaaaaatacagtcaacacaagtacaaattcgtagcaaagaagtactctccaccatcaattccagcagagtttttcgaagatccaagcagctacaagcctgattaa

SEQ ID NO: 51 - protein, 356 aa, Oryza sativa
MSKEDAMSTGDSTESLDIDCLDDGDSEVVSSLQHLADDKLHISDNRDVAGVASKWTKHGCNSVEIDYIVDIDNNHEDPQLCATLAFDIYKHLRVAETKKRPSTDFVETIQKNIDTSMRAVLIDWLVEVTEEYRLVPETLYLTVNYIDRYLSSKVINRRKMQLLGVACLLIASKYEEICPPQVEELCYISDNTYTKDEVLKMEASVLKYLKFEMTAPTTKCFLRRFLRAAQVCHEAPVLHLEFLANYIAELSLLEYSLICYVPSLIAASSIFLAKFILKPTENPWNSTLSFYTQYKPSDLCNCAKGLHRLFLVGPGGNLRAVREKYSQHKYKFVAKKYSPPSIPAEFFEDPSSYKPD

SEQ ID NO: 52 - DNA, 2046 bp, Oryza sativa
  source 1..2046: mol_type = genomic DNA
atggccttggtgtgtgcggaatgccgtcttgttgacatcgtccgggtgcatgcgaacctgatggtacccgaaatggagatccagcttggagaagaagtgggcgtcgcaaagttcatcaagcaattcgtcgagaaccaaaatgggaaacatgtccttggcagtcaccgtgttgagggctcggtagtccacgtagaagcgccatggcttgtggaccaacagaaccgacgacgagaatggcgacgtgttggggcagatggagagcttgtcagcgcattatttctccaattcgtcgttttgaagctgtgggtagcgatacggtcgaaccactaccgggggcttgccgggcttgaggtggatgtgatgattgatggatcgggtcggtgggaggccggttggagcgcgaagaggtcgtcaaattccgccagcaaggacggaaggagatcgtcggcgtgcagtgcagcgcagctgaggtcgagggcgaagcagtcgacgtcgaacacctcggtggccacctacaggcgaagcccgtgcacgacacccgcgccgcggatgcgatcgttgtcagccaccaccacgcggagaccttggcgtaccggttggagaggtaggccgatgcgctgggccagcttggtggtgatgaaattgtgtgttgaagccggagtcaacaagggccatcacctgctcggccgcgatacgcgccacgaggcacatcgtgctagtgccattgacaccgaaaatcgcgttgagcgagatgtgtggttcatcggtgtcgtctgtgtcgtcccattcgctgaaattagccgtggtgtcgtcgtactcaagaggccttgtttacagcgctctggtaactctgctggcgagagacaacgaaattggtgtcctggcacgagccaaggccatggcctcctggaaatcctcgggctgttggagctcgacatcgatgcagatgtcgtcggtgagccccgctgtgaagagctgaacttgttggcggtcggtgaggaggtcggacgtgcggctacccaacgccaaaaggcgctgctggtacgtcgtcaccgtgccaacctgagtgagatgcttgagttcacccagggaattgttgcggatcggcggtccatactgaccgtaacagagctgcttgaacgtatcccaatccggaggacccatgtgctgctcataatggaagtaccattcctgtgcagctcaagtgagatgaccaggaaacgtccatcaactgattttatggaaacaatccaaaaggatgtaaacccaagcatgagagcgatcctgatagactggcttgtggaagtcgctgaagaatatcgtcttgttcctgatacattatacctgacagttaactacattgatcgttatctttctggcaatgagatcaatcgtcaaagactgcaattacttggagttgcttgtatgcttattgctgcaaaatacgaggagatatgtgcacctcaagtagaagaattctgctatataactgacaacacatacttcagagatgagtgctggaatgaatcgaactctaataactctcttattgcctacaacaggagatttgtccgtgttgcacaagtatcggatgagcttttcatcgtgcaggatccagcattgcatcttgagttcctagccaattatgttgctgagctatcactgctggagtacaatctactttcttaccctccttcactagtagcggcatcggctattttcttggccaaattcatactgcagccaacaaagcacccttggaattccacccttgctcactacacacaatacaagtcgtcagagttaagcgactgtgtaaaggcattgcaccgcctttttagcgttggtcccgggagtaaccttcctgcaatcagggagaagtatacccaacataagatactgcatgcagctgatgtgatcgacttgaacatggcaaatgcatttaagaatgtgaaaatattatgtcaatgtccctgtcaatgcaaccttcttgaagaagtcatgctcaagctatttccatactggaagctaagcacagctgtttag

SEQ ID NO: 53 - protein, 681 aa, Oryza sativa
MALVCAECRLVDIVRVHANLMVPEMEIQLGEEVGVAKFIKQFVENQNGKHVLGSHRVEGSVVHVEAPWLVDQQNRRREWRRVGADGELVSALFLQFVVLKLWVAIRSNHYRGLAGLEVDVMIDGSGRWEAGWSAKRSSNSASKDGRRSSACSAAQLRSRAKQSTSNTSVATYRRSPCTTPAPRMRSLSATTTRRPWRTGWRGRPMRWASLVVMKLCVEAGVNKGHHLLGRDTRHEAHRASAIDTENRVERDVWFIGVVCVVPFAEISRGVVVLKRPCLQRSGNSAGERQRNWCPGTSQGHGLLEILGLLELDIDADVVGEPRCEELNLLAVGEEVGRAATQRQKALLVRRHRANLSEMLEFTQGIVADRRSILTVTELLERIPIRRTHVLLIMEVPFLCSSSEMTRKRPSTDFMETIQKDVNPSMRAILIDWLVEVAEEYRLVPDTLYLTVNYIDRYLSGNEINRQRLQLLGVACMLIAAKYEEICAPQVEEFCYITDNTYFRDECWNESNSNNSLIAYNRRFVRVAQVSDELFIVQDPALHLEFLANYVAELSLLEYNLLSYPPSLVAASAIFLAKFILQPTKHPWNSTLAHYTQYKSSELSDCVKALHRLFSVGPGSNLPAIREKYTQHKILHAADVIDLNMANAFKNVKILCQCPCQCNLLEEVMLKLFPYWKLSTAV

SEQ ID NO: 54 - DNA, 1473 bp, Oryza sativa
  source 1..1473: mol_type = genomic DNA
atggctggaaggaaggaaaatccggtgcttactgcttgccaagcacccagtggtcgaatcacacgagctcaagctgctgcaaatcgtggacggtttgggtttgctccctccgtatcactacccgcaagaactgaacgaaagcagacagcaaaaggaaagacaaaaaggggagctttggatgaaatcactagtgcaagtactgcaacttcagctcctcagcctaaacggcgcacagtgctcaaggatgtaaccaacatcggctgtgccaactcatccaaaaattgcaccaccacgagcaagctgcagcaaaagtcaaagcccacccaaagggtgaaacaaatcccgagcaaaaagcagtgtgcaaagaaggttcctaagctaccccctccggctgttgctggaacttcatttgtgattgattctaaaagttctgaagaaactcaaaaggtggagcttttggcaaaagcagaggaacccacaaatttgtttgaaaacgaggggttactgtcattgcagaatattgagcgaaacagggacagtaattgccatgaggcattctttgaggcaagaaacgccatggataaacatgaactcgctgactccaagcctggtgactctagtggtttaggttttatagatattgacaatgataatggaaatcctcaaatgtgtgcttcctatgcttcagagatatacacaaatctgatggcctctgagcttatcagaagacccaggtcaaattacatggaggctttgcaacgtgacatcacaaagggcatgcgaggcattctcattgattggcttgttgaggtttctgaagaatataagcttgtgccagacacactctacctaaccattaatcttattgaccgatttctttctcaacattatattgaaagacagaaactccaacttcttggaataacaagcatgctgattgcctcgaaatatgaagagatatgtgctcctcgtgttgaagaattttgtttcataactgacaatacatacacaaaagctgaggtgctgaaaatggagggcctggtgcttaatgatatggggtttcatctatctgttccaacaacaaaaacatttctcaggagattccttagagccgcacaggcttctcgtaatgttccttcaattaccttgggatatctggccaattatcttgcagagctgaccctgatcgattacagtttcctcaaatttcttccttcagtggtggcagcatctgcagtctttcttgcaagatggacacttgaccaatctgacattccatggaatcatactcttgagcactacacttcttacaaaagctctgatattcaaatatgtgtctgtgctctacgggaactgcagcataacaccagtaattgccctctcaatgctatacgtgaaaagtataggcaacaaaagtttgagtgtgtagccaacctgacatcaccggagctggggcagtcactcttcagctga

SEQ ID NO: 55 - protein, 490 aa, Oryza sativa
MAGRKENPVLTACQAPSGRITRAQAAANRGRFGFAPSVSLPARTERKQTAKGKTKRGALDEITSASTATSAPQPKRRTVLKDVTNIGCANSSKNCTTTSKLQQKSKPTQRVKQIPSKKQCAKKVPKLPPPAVAGTSFVIDSKSSEETQKVELLAKAEEPTNLFENEGLLSLQNIERNRDSNCHEAFFEARNAMDKHELADSKPGDSSGLGFIDIDNDNGNPQMCASYASEIYTNLMASELIRRPRSNYMEALQRDITKGMRGILIDWLVEVSEEYKLVPDTLYLTINLIDRFLSQHYIERQKLQLLGITSMLIASKYEEICAPRVEEFCFITDNTYTKAEVLKMEGLVLNDMGFHLSVPTTKTFLRRFLRAAQASRNVPSITLGYLANYLAELTLIDYSFLKFLPSVVAASAVFLARWTLDQSDIPWNHTLEHYTSYKSSDIQICVCALRELQHNTSNCPLNAIREKYRQQKFECVANLTSPELGQSLFS

SEQ ID NO: 56 - DNA, 1527 bp, Oryza sativa
  source 1..1527: mol_type = genomic DNA
atgtcgagcaacctagcagcctcccgccgctcgtcgtcgtcgtcctcggtggcggcggcggcggcggcgaagcgacccgcggtgggggagggaggaggaggaggaggagggaaggcggcagcgggcgccgccgcggcaaagaagcgcgtggcgcttagcaacatcagcaacgtcgccgctggtggtggcgccccagggaaggccggcaatgcgaagttgaatttagctgcctcagctgcaccagtgaagaagggatctttggccagtggccgcaatgtgggcacgaatcgggcctcggcggtgaaatcggcttccgccaagccggctccggccatatcccgccatgagagcgccacacagaaggagtctgttcttcctcctaaagtgcctagcattgtgccgactgctgcactggcacctgtcactgtaccctgcagcagcttcgtctcccctatgcattcaggagattcagtttcggttgacgagacgatgtcgacgtgtgactcaatgaaaagcccagaatttgagtacattgataatggggattcctcctcagttctaggttccttgcagcgaagagcaaacgaaaacctgcgtatctcagaggatagagatgtcgaagaaactaagtggaagaaggatgctccttccccaatggaaatcgaccaaatttgtgatgttgacaataactacgaggatccgcagttgtgtgctactcttgcttctgatatctacatgcacttgcgcgaggctgagaccaggaaacgtccatcaactgattttatggaaacaatccaaaaggatgtaaacccaagcatgagagcgatcctgatagactggcttgtggaagtcgctgaagaatatcgtcttgttcctgatacattatacctgacagttaactacattgatcgttatctttctggcaatgagatcaatcgtcaaagactgcaattacttggagttgcttgtatgcttattgctgcaaaatacgaggagatatgtgcacctcaagtagaagaattctgctatataactgacaacacatacttcagagatgaggttttggaaatggaagcttctgtcctgaattacctgaagtttgaagtgactgcacctacagcaaaatgctttttgaggagatttgtccgtgttgcacaagtatcggatgaggatccagcattgcatcttgagttcctagccaattatgttgctgagctatcactgctggagtacaatctactttcttaccctccttcactagtagcggcatcggctattttcttggccaaattcatactgcagccaacaaagcacccttggaattccacccttgctcactacacacaatacaagtcgtcagagttaagcgactgtgtaaaggcattgcaccgcctttttagcgttggtcccgggagtaaccttcctgcaatcagggagaagtatacccaacataagtacaaatttgtggcgaagaagccctgcccaccctcaataccgaccgaattctttcgcgacgcaacatgctga

SEQ ID NO: 57 - protein, 508 aa, Oryza sativa
MSSNLAASRRSSSSSSVAAAAAAKRPAVGEGGGGGGGKAAAGAAAAKKRVALSNISNVAAGGGAPGKAGNAKLNLAASAAPVKKGSLASGRNVGTNRASAVKSASAKPAPAISRHESATQKESVLPPKVPSIVPTAALAPVTVPCSSFVSPMHSGDSVSVDETMSTCDSMKSPEFEYIDNGDSSSVLGSLQRRANENLRISEDRDVEETKWKKDAPSPMEIDQICDVDNNYEDPQLCATLASDIYMHLREAETRKRPSTDFMETIQKDVNPSMRAILIDWLVEVAEEYRLVPDTLYLTVNYIDRYLSGNEINRQRLQLLGVACMLIAAKYEEICAPQVEEFCYITDNTYFRDEVLEMEASVLNYLKFEVTAPTAKCFLRRFVRVAQVSDEDPALHLEFLANYVAELSLLEYNLLSYPPSLVAASAIFLAKFILQPTKHPWNSTLAHYTQYKSSELSDCVKALHRLFSVGPGSNLPAIREKYTQHKYKFVAKKPCPPSIPTEFFRDATC

SEQ ID NO: 58 - DNA, 1158 bp, Oryza sativa
  source 1..1158: mol_type = genomic DNA
atggctgacaaggagaactccaccccggcctccgcggcgcggctcacccgctcgtctgcggcggctggggcgcaggccaagcgttcggccgccgcgggcgtcgccgacggtggcgcgccgccggcgaagaggaagcgcgtcgcgctcagcgacctcccgaccctctccaacgccgtcgtcgtcgccccccgccagccgcaccaccccgtcgtcatcaagccgtcgtccaagcagcccgagcccgccgcggaggcggcggcgcccagcggcggcggcggcggctcccccgtgtcatccgcgtcgacgtcgacggcgtcgccctcctccggttgggacccgcagtacgcctccgacatctacacctacctccgatccatggaggtggaggcgcggaggcagtcggcggcggactacatcgaggcggtgcaggtggacgtgacggcgaacatgcgggccatcctcgtggactggctggtggaggtcgccgacgagtacaagctcgtcgccgacacgctctacctcgccgtctcctacctcgaccgctacctctccgcccacccgctcaggcgcaacaggctgcagctcctcggcgtcggcgccatgctcatcgctgcgaagtacgaggagattagccctcctcatgtggaggatttctgctacatcactgataatacgtacactaggcaggaggttgtcaagatggagagcgacatactcaagcttctcgagttcgagatgggcaatcctaccatcaagacattcctcaggcggttcacgagatcttgccaggaagacaaaaagcgctccagcttgttattggagttcatggggagttatcttgctgagcttagtctacttgactacggctgtctccggttcttgccatcggtggttgctgcctcagtggtgtttgttgctaaactgaacattgatccgtacaccaatccttggagcaagaagatgcagaagttgacaggatacaaggtgtctgaactgaaggattgcatcttggccattcatgacttgcagctcagaaaaaaatgttcaaacttaactgcaattcgcgacaagtacaagcaacacaagttcaagtgtgtctcaacattgcttccccctgttgatatccctgcgtcatacctccaagatttaacagagtag

SEQ ID NO: 59 - protein, 385 aa, Oryza sativa
MADKENSTPASAARLTRSSAAAGAQAKRSAAAGVADGGAPPAKRKRVALSDLPTLSNAVVVAPRQPHHPVVIKPSSKQPEPAAEAAAPSGGGGGSPVSSASTSTASPSSGWDPQYASDIYTYLRSMEVEARRQSAADYIEAVQVDVTANMRAILVDWLVEVADEYKLVADTLYLAVSYLDRYLSAHPLRRNRLQLLGVGAMLIAAKYEEISPPHVEDFCYITDNTYTRQEVVKMESDILKLLEFEMGNPTIKTFLRRFTRSCQEDKKRSSLLLEFMGSYLAELSLLDYGCLRFLPSVVAASVVFVAKLNIDPYTNPWSKKMQKLTGYKVSELKDCILAIHDLQLRKKCSNLTAIRDKYKQHKFKCVSTLLPPVDIPASYLQDLTE

SEQ ID NO: 60 - DNA, 1122 bp, Oryza sativa
  source 1..1122: mol_type = genomic DNA
atggccggcaaggagaacgccgcggcggcgcagccccgcctcacccgcgccgccgccaagcgcgcggccgccgtcaccgccgtggccgtcgccgccaagcgcaagcgcgtcgcgctcagcgagctccccacgctgtccaacaacaacgccgtggtgctcaagccgcagccggcgcccaggggcggcaagagggccgcctcccacgccgccgagcccaagaagccagctccgccgccggcgccggcggtggtggtcgtggtcgacgacgacgaggagggggagggggatccgcagctctgcgcgccctacgcctccgacatcaactcctacctccgctccatggaggtgcaagcgaagcggcggccggcggcggactacatcgagacggtgcaggtggacgtgacggccaacatgcgaggcatcctggtcgactggctcgtcgaggtcgccgaggagtacaagctcgtctccgacacgctctacctcaccgtctcctacatcgaccgcttcctctccgccaaatccatcaaccgccagaagctgcagctcctcggcgtctccgccatgctcatcgcctcgaagtatgaggagatcagccccccaaatgtggaggatttctgctatataaccgacaatacctatatgaaacaggaggttgtcaagatggagcgcgatatactgaatgttctcaagtttgagatgggcaatcctacaaccaagacgttcctgaggatgttcatcagatctagccaagaagacgataagtatcctagccttcccttggagttcatgtgtagctatcttgccgagctgagcctgctggagtacggctgtgttcggctcttgccatccgttgttgcagcctcagtggtgtttgttgcaaggctaacccttgattcagacaccaatccttggagcaagaagttgcaagaggtgaccggctacagggcatctgagttgaaggattgcattacctgcatacatgacttgcagctaaacaggaaagggtcatctctaatggctatccgggacaagtacaagcaacataggttcaagggcgtatcaacattgttaccccctgttgagatccctgcatcatacttcgaagacctaaacgagtag

SEQ ID NO: 61 - protein, 373 aa, Oryza sativa
MAGKENAAAAQPRLTRAAAKRAAAVTAVAVAAKRKRVALSELPTLSNNNAVVLKPQPAPRGGKRAASHAAEPKKPAPPPAPAVVVVVDDDEEGEGDPQLCAPYASDINSYLRSMEVQAKRRPAADYIETVQVDVTANMRGILVDWLVEVAEEYKLVSDTLYLTVSYIDRFLSAKSINRQKLQLLGVSAMLIASKYEEISPPNVEDFCYITDNTYMKQEVVKMERDILNVLKFEMGNPTTKTFLRMFIRSSQEDDKYPSLPLEFMCSYLAELSLLEYGCVRLLPSVVAASVVFVARLTLDSDTNPWSKKLQEVTGYRASELKDCITCIHDLQLNRKGSSLMAIRDKYKQHRFKGVSTLLPPVEIPASYFEDLNE

SEQ ID NO: 62 - DNA, 1920 bp, Arabidopsis thaliana
  source 1..1920: mol_type = genomic DNA
atgagtagtacgatgttcgtgaaacggaatccgattagagaaaccaccgccgggaaaatctcttcgccgtcgtcaccgactttgaatgttgcagtcgcgcatataagagctggatcttattacgaaatcgatgcttcgattcttcctcagagatcgccggaaaatcttaaatcgattagagtcgtcatggtgagcaaaatcacggcgagtgacgtgtctctccggtacccaagcatgttttcactccgatcgcatttcgattacagtaggatgaaccggaataaaccgatgaagaagaggagtggtggtggtcttcttcctgttttcgacgagagtcatgtgatggcttcggagctagctggagacttgctttacagaagaatcgcacctcatgaactttctatgaatagaaattcctggggtttctgggtttctagttcttctcgcaggaacaaatttccaagaagggaggtggtttctcaaccggcgtacaatactcgtctctgtcgcgctgcttcaccggagggaaagtgctcgtctgagctgaaatcgggagggatgatcaagtggggaaggagattgcgtgtgcagtatcagagtcggcatattgatactaggaagaataaggaaggtgaggagagttctagagtgaaggatgaagtttacaaagaagaagagatggagaaagaagaggatgatgatgatgggaatgaaataggaggcactaaacaagaggcaaaggagataactaatggaaatcgtaagagaaagctgattgaatcaagtactgagagactcgctcagaaagctaaggtttatgatcagaagaaggaaactcaaattgtggtttataagaggaaatcagagaggaagttcattgatagatggtctgttgagaggtacaaactagctgagaggaacatgttaaaagtgatgaaggagaagaatgcagtgtttggcaactccatactcaggccagagttgaggtcagaagcaaggaagctgattggtgacacaggtctattggatcatctgcttaagcacatggctggtaaggtggctcctggaggtcaagataggtttatgagaaagcacaatgcagatggggcaatggagtattggttggagagttctgatttgattcacataaggaaagaagcaggagttaaagatccttactggactcctccacctggttggaagcttggtgacaacccttctcaagatcctgtctgcgctggagaaatccgtgacatcagagaagaattagctagcctgaaaagagaattgaagaaacttgcgtcaaagaaggaagaggaggagcttgttatcatgactacgcctaattcttgtgttactagtcagaatgataatctgatgactccagcaaaggaaatctacgctgatctgctgaaaaagaaatacaaaattgaggaccagctagtgattattggagaaaccttgcgtaaaatggaggaagacatgggatggcttaagaaaacagtggacgagaactatcctaaaaagccagactcaacagagacacctttgctactagaggattcaccaccaatacagacactagaaggagaagtgaaggtggtgaacaagggtaaccaaatcacagagtcacctcaaaacagagaaaaaggaaggaagcatgatcaacaagaaagatcaccactttcactaataagcaacactggtttcagaatctgcaggcctgtggggatgttcgcatggccccaattgcctgctcttgctgctgctactgatactaatgcttcttcgccaagtcacagacaagcctacccatccccttttccagtcaagccacttgcagctaagcgtcctcttggcttgacgtttcccttcaccatcatacccgaagaagctcccaagaatctcttcaacgtttga

SEQ ID NO: 63 - protein, 639 aa, Arabidopsis thaliana
MSSTMFVKRNPIRETTAGKISSPSSPTLNVAVAHIRAGSYYEIDASILPQRSPENLKSIRVVMVSKITASDVSLRYPSMFSLRSHFDYSRMNRNKPMKKRSGGGLLPVFDESHVMASELAGDLLYRRIAPHELSMNRNSWGFWVSSSSRRNKFPRREVVSQPAYNTRLCRAASPEGKCSSELKSGGMIKWGRRLRVQYQSRHIDTRKNKEGEESSRVKDEVYKEEEMEKEEDDDDGNEIGGTKQEAKEITNGNRKRKLIESSTERLAQKAKVYDQKKETQIVVYKRKSERKFIDRWSVERYKLAERNMLKVMKEKNAVFGNSILRPELRSEARKLIGDTGLLDHLLKHMAGKVAPGGQDRFMRKHNADGAMEYWLESSDLIHIRKEAGVKDPYWTPPPGWKLGDNPSQDPVCAGEIRDIREELASLKRELKKLASKKEEEELVIMTTPNSCVTSQNDNLMTPAKEIYADLLKKKYKIEDQLVIIGETLRKMEEDMGWLKKTVDENYPKKPDSTETPLLLEDSPPIQTLEGEVKVVNKGNQITESPQNREKGRKHDQQERSPLSLISNTGFRICRPVGMFAWPQLPALAAATDTNASSPSHRQAYPSPFPVKPLAAKRPLGLTFPFTIIPEEAPKNLFNV

SEQ ID NO: 64 - DNA, 1254 bp, Oryza sativa
  source 1..1254: mol_type = genomic DNA
atgaccgccgccgccgtcaacgcctcgcgcgtcatgcgccgccgcgccgccggcgaggacctgggcgacggcgacttctgggccggtggcgccccgcgcctctacgacttctcccagcaggagcagaagccgttcttgccggcgccgccgtcgcccgcgcccgtcccggcgtcgccgccgtcccccgcggcggagtcggtggcgccgtgcctcctcacgctgcagtgcagcggcgtcgggtggggggtcaggaagcgggtcaggtacgtcgggaggcaccaccacctcgcgcgccaccacgctcccgaacgcgccgtggacgccgcgcgggacgacgacgaggcgagctccgccaaggccaagaacgagagcccgaaggaggaggcggcggcggcagaagaagacgacgacgtcgaacacaaggtggcggtgcgcaccacgtcggaggagaagaagaagaagaggaggaggaagcgcggccgtggccgtgtccgtggccatggcgtcgccaagcgtcccaagaaggaggatgaggaggggacgaagctctcggctcccaaggccgagcagctcgaggaggaggaggagggcgccgcggtggcggcgccgagcggcatgatcgaccggtggaaggcgacccggtacgccaccgcggaggcgtcgctgctcgccatcatgcgcgcccacggcgcgcgcgccgggaagcccgtcccgcgcgcggcgctgcgggaggaggcgcgcgcccacatcggcgacacgggcctcctcgaccacctcctcaggcacatcgccgacaaggtggcgcccggcggcgccgagcggttccggcggcggcacaacgccggcggcgggctggagtactggctggagcccgccgagctcgccgccgtgcggcggaaggccggcgtggccgacccgtactgggtgcctcctcccggatggaagccaggggaccccgtgtcgccggagggctacttgctggaggtgaggaagcaggtggagcagctcgccgttgagctcgccggcgtcagaaggcacatggatcacctcacttccaatgtgagtcaagtgggcaaggaaatcaaatctgaggctgagaagtcctacaatacatgtcagggtggggacccaccctaccttgaccggatctcgatccgtgccttcgcccggaagctaagccgcagctcgtccttgagcgggacccagcgccgtcccccgcctgacacgggggacagccctgtcattccctcataa

SEQ ID NO: 65 - protein, 417 aa, Oryza sativa
MTAAAVNASRVMRRRAAGEDLGDGDFWAGGAPRLYDFSQQEQKPFLPAPPSPAPVPASPPSPAAESVAPCLLTLQCSGVGWGVRKRVRYVGRHHHLARHHAPERAVDAARDDDEASSAKAKNESPKEEAAAAEEDDDVEHKVAVRTTSEEKKKKRRRKRGRGRVRGHGVAKRPKKEDEEGTKLSAPKAEQLEEEEEGAAVAAPSGMIDRWKATRYATAEASLLAIMRAHGARAGKPVPRAALREEARAHIGDTGLLDHLLRHIADKVAPGGAERFRRRHNAGGGLEYWLEPAELAAVRRKAGVADPYWVPPPGWKPGDPVSPEGYLLEVRKQVEQLAVELAGVRRHMDHLTSNVSQVGKEIKSEAEKSYNTCQGGDPPYLDRISIRAFARKLSRSSSLSGTQRRPPPDTGDSPVIPS

SEQ ID NO: 66 - DNA, 1914 bp, Oryza sativa
  source 1..1914: mol_type = genomic DNA
atgaccgccgccgccgtcaacgcctcgcgcgtcatgcgccgccgcgccgcaggcgaggacctgggcgacggcggcgatggcgacggcgacttctgggccggtggcgccccccgcctctacgacttctcccagcaggagcagaagccgttcttgcccgcgcccgcgcccgcgccgccgtcgcccgcgcccgtcccggcgtcgccgccgtcccccgcggcggagtcggtggcgccgtgcctcctcacgctgcagtgcagcggcgtcgggtggggtgtcaggaagcgggtccggtacgtcgggaggcaccaccacctcgcgcgccaccacgctcccgagcgcgccgtggacgccgcgcgggacgacgacgaggcgagctccgccaaggccaagaacgagagcccgaaggaggaggcagcggcggcagaagaagacgacgacaacgtcgaacacaaggtggcggtgcccaccacgtcggaggagaagaagaggaggaggaggaggaagcgtggccgtggccgtgtcggtggccatggcgtcgccaagcgtcccaagaaggaggaggaggaggaggagacgaagctctcggctcccaaggccgagcagctcgaggaggaggagggcgccgcggtggcggcgccgagcggcatgatcgaccggtggaaggcgacccggtacgccaccgcggaggcgtcgctgctcgccatcatgcgcgcccgcggcgcgcgcgccgggaagcccgtcccgcgcggggcgctgcgggaggaggcgcgcgcccacattggcgacacgggcctcctcgaccacctcctcaggcacatcgccgacaaggtggcgcccggcggcgccgagcggttccggcggcggcacaacgccggcggcgggctggagtactggctcgagcccgccgagctcgccgccgtgcggcggaacgccggcgtggccgacccgtactgggtgcctcctcccggatggaagccaggggaccccgtctcgccggagggctacttgctggaggtgaggaagcaggtggagaagctcgccgtggagctcgccggcgtcagaaggcacatggatcacctctcttccaatgtgagtcaagtgggcaaggaaatcaaatctgaggctgagaaatcctacaatacatgccaggagaagtatgcctgtatggagaaagccaatggcaatctggaaaagcagcttctgtccttggaggagaagtatgagaatgcaacacacgcaaatggcgagctgaaggaggagttgttgtttctcaaggagaagtttgtgagtgtggtcgagaacaacaccagactggagcaccagctgactgctttatccacttctttcctgtctctaaaggaggaactgctctggctggaaaaagaagaagctgatctgtatgtcaaggaaccatgggaagacgacgatgaaaagcaagaacacgatgccgggaaagaggcgaaggacgacgatgtcgccggcgtcagtgcagccaacgaccagccggacgtcgacggcgatggcaccaccaccaccaccaccaccagcagcaatggtggcagcgggaagagaacatcgaggaagtgcagcgtgcgcatctccaagccgcagggcgcgttccagtggccgacgccgagcctgccgttctcgccggagctcgccgcgccgccgtcgccgccgctgaccccgacggcgcccgtcgtcgccggcgccgccaacttcgccaccatggacgagctctacgagtacatgatggccggcggcctccccacgccaccgtccaccaccagcaacgccgggaagctcccctcgctgcccgccgccacggcctgcgccacgacgccgccggtgaagacggcggacgccgccggcgacgtgggcaccgagctggcactggccactcccgcctactga

SEQ ID NO: 67 - protein, 637 aa, Oryza sativa
MTAAAVNASRVMRRRAAGEDLGDGGDGDGDFWAGGAPRLYDFSQQEQKPFLPAPAPAPPSPAPVPASPPSPAAESVAPCLLTLQCSGVGWGVRKRVRYVGRHHHLARHHAPERAVDAARDDDEASSAKAKNESPKEEAAAAEEDDDNVEHKVAVPTTSEEKKRRRRRKRGRGRVGGHGVAKRPKKEEEEEETKLSAPKAEQLEEEEGAAVAAPSGMIDRWKATRYATAEASLLAIMRARGARAGKPVPRGALREEARAHIGDTGLLDHLLRHIADKVAPGGAERFRRRHNAGGGLEYWLEPAELAAVRRNAGVADPYWVPPPGWKPGDPVSPEGYLLEVRKQVEKLAVELAGVRRHMDHLSSNVSQVGKEIKSEAEKSYNTCQEKYACMEKANGNLEKQLLSLEEKYENATHANGELKEELLFLKEKFVSVVENNTRLEHQLTALSTSFLSLKEELLWLEKEEADLYVKEPWEDDDEKQEHDAGKEAKDDDVAGVSAANDQPDVDGDGTTTTTTTSSNGGSGKRTSRKCSVRISKPQGAFQWPTPSLPFSPELAAPPSPPLTPTAPVVAGAANFATMDELYEYMMAGGLPTPPSTTSNAGKLPSLPAATACATTPPVKTADAAGDVGTELALATPAY

SEQ ID NO: 68 - DNA, 2412 bp, Oryza sativa
  source 1..2412: mol_type = genomic DNA
atggacgcggagatggcggctcctgcgcttgcggcagctcatctgctggactcgcccatgaggccacaggtgagcagatactactccaagaagaggggtagcagccacagcagaaatggcaaggatgatgccaaccatgacgagtccaagaaccaatcacccggcttgcccctgagcagacagagcctgtcctcatctgccacccacacctaccacaccggagggttctacgagatcgaccacgagaagcttccccccaaatccccaattcatctcaagtccatacgcgtggtaaaggtgagcggctacacaagcctggacgtcacagtgagcttcccgtccctcctggcgctgcgaagcttcttctcctcctccccacggtcgtgcactgggccggagctcgacgagcgcttcgtcatgagcagcaaccacgcggcccgcatcctgcgccgtcgggtggccgaggaggagctcgcgggcgacgtgatgcaccaggacagcttctggctcgtcaagccctgcctctatgacttctccgcgtcgtcaccacatgatgtgctgaccccgtcgccgccgcctgccacagcgcaggcgaaggcgccggcagccagttcctgccttctcgacaccttgaagtgcgacggcgccgggtggggcgtgaggcgccgtgtcaggtacattggtcgccaccacgatgcttccaaggaggccagcgctgccagcctcgatggctacaacacagaggtcagcgtccaggaggagcagcagcagcgactgcggcttcgactgcggttgcgacaacgccgggagcaggaagacaacaagagcactagcaatggcaagaggaagcgggaggaggcagagagcagcatggacaagagcagagccgccaggaagaagaaagccaagacttacaagagtcccaagaaggtggagaagaggcgcgtcgtggaggctaaagacggcgaccctcggcgcggcaaggaccggtggtcggccgagcggtacgcagcggcggagaggagcctgctggatataatgcgctcccatggtgcctgcttcggtgcgccggtgatgcggcaggctctgcgggaggaagcccgcaagcatatcggtgacaccggcctccttgaccacctgctcaagcacatggccggcagggtaccggaaggcagcgcggaccggttccgtcgccggcacaatgcggatggtgccatggagtactggctggagccggcggagcttgccgaggtacggcggctggctggagtgtctgatccatactgggtgccgccacctgggtggaagccaggtgatgacgtgtccgcagtcgccggtgacctcctggtcaagaagaaggtggaagagctcgctgaggaggttgatggtgtaaaaaggcacatcgagcagctcagttctaatttggtgcagctggagaaggaaacaaaatctgaggcagagcgatcttacagctctaggaaggagaagtatcagaagttgatgaaggcaaatgaaaagctcgagaaacaggtgttatctatgaaggacatgtatgagcatctggttcagaaaaagggtaagctgaagaaggaggtgctgtccttgaaggataaatacaagcttgtgctggagaagaatgataaactggaggaacagatggctagtctctccagctccttcctttctttgaaggaacaattgctgctgccaagaaatggagataatctgaacatggaaagggaaagggtggaagtgactttgggcaagcaagaaggccttgttcccggcgaaccactgtatgttgatggtggtgaccggatcagccagcaagcagatgccaccgtcgtccaagtcggcgagaagaggacggcgaggaagagcagcttccgcatctgcaagccacagggaacgttcatgtggccacacatggcgtctggcacgagcatggccatcagtgggggaggcagcagcagctgccctgtcgcctccgggccagagcagctccctcgcagcagcagctgccccagcattgggcctggtggcctcccgccgtcgtcacgagccccagccgaggtggtggtcgcgtcgccactggacgagcacgtggcgttccgcgggggcttcaacacgccgccctcggcatcgtccaccaacgccgccgctgccgccaagctgcctcccctgcccagcccgacgtcacctctccagacacgggccctgttcgccgctggcttcactgtcccggcattacacaacttctccggcctcaccttacgccatgtggactcctcgtcgccgtcgtccgcgccatgcggtgctagggagaagatggtgaccctgttcgatggagactgccgggggatcagcgtcgtgggcaccgagctggcactggccactccgtcctactgctga

SEQ ID NO: 69 - protein, 803 aa, Oryza sativa
MDAEMAAPALAAAHLLDSPMRPQVSRYYSKKRGSSHSRNGKDDANHDESKNQSPGLPLSRQSLSSSATHTYHTGGFYEIDHEKLPPKSPIHLKSIRVVKVSGYTSLDVTVSFPSLLALRSFFSSSPRSCTGPELDERFVMSSNHAARILRRRVAEEELAGDVMHQDSFWLVKPCLYDFSASSPHDVLTPSPPPATAQAKAPAASSCLLDTLKCDGAGWGVRRRVRYIGRHHDASKEASAASLDGYNTEVSVQEEQQQRLRLRLRLRQRREQEDNKSTSNGKRKREEAESSMDKSRAARKKKAKTYKSPKKVEKRRVVEAKDGDPRRGKDRWSAERYAAAERSLLDIMRSHGACFGAPVMRQALREEARKHIGDTGLLDHLLKHMAGRVPEGSADRFRRRHNADGAMEYWLEPAELAEVRRLAGVSDPYWVPPPGWKPGDDVSAVAGDLLVKKKVEELAEEVDGVKRHIEQLSSNLVQLEKETKSEAERSYSSRKEKYQKLMKANEKLEKQVLSMKDMYEHLVQKKGKLKKEVLSLKDKYKLVLEKNDKLEEQMASLSSSFLSLKEQLLLPRNGDNLNMERERVEVTLGKQEGLVPGEPLYVDGGDRISQQADATVVQVGEKRTARKSSFRICKPQGTFMWPHMASGTSMAISGGGSSSCPVASGPEQLPRSSSCPSIGPGGLPPSSRAPAEVVVASPLDEHVAFRGGFNTPPSASSTNAAAAAKLPPLPSPTSPLQTRALFAAGFTVPALHNFSGLTLRHVDSSSPSSAPCGAREKMVTLFDGDCRGISVVGTELALATPSYC

SEQ ID NO: 70 - DNA, 495 bp, synthetic construct
  source 1..495: mol_type = other DNA
gagatttgggaaatgtgcaatttgggtttatctggttttgttgttttggttatttagttttgagtccggtttgaagaaatgcttcatagatatataaatgtaaccagaaatataaataaatcataactaagtagtattatatttttgttaagcttaattatttcaattccaagtctttcctaagaatttgttgaaaatttataatttacgttacactttgtaaaatcagaacgatccaattcacaagattaatgctacgactctgtttttttctttaaaaataaatcaataatcatctccactaaacctctcaataacttagcagtcttaatgaaatttaaagctaatctatcaatcacattctcaccacgtcgccaaactcgttgccgtttcaatcttttcaagttccctttcatgctgtttataaacccttgcactctttcactcacagactcactacaagtctacaccacaaacttaccaaatcatccaaaa

SEQ ID NO: 71 - DNA, 1890 bp, synthetic construct
  source 1..1890: mol_type = other DNA
acacgttatggaaagcaaagaataacaaaagtaatattcttacctatcttttttagttggaaaacttgcattgtgtaacgtattcaaacattttcgaaatggtttattggtttttgtatataattaatttgggttaaactgatatattttatgagataaatatagaatctcatgtgcttatacaaagcaactatattaattttgttaaccgtaagttacaaaaacagtggtcggaggaaatcaggaaaataaaaagagagaaaagagtctacacaatgggccaattattataagtaaatgatagtcatgaaagcccatttcagaagaagatcttttggaaatgagaatagtgctctaggctcactggtcctttttactattggtatagaaactgtcaaagcccaacaggtttaaactagcatttcaggcgctgtaattcttctgcagtttgtttgtataaacttggaatatggatgggttaaacactgatatttcttcactcgttttgtctcacatactgttcgatgcttaacacaggtcttaaaaagaaactgggtttgatgtctcaaatctactcaagaaagaaagatatcttgagtttgcatcgagacagaaaagagtacgactatacatagctgctggggtagtagcctgcagagaatacagattttgaaccacgtacaaggaaccaaatcagtgtatgtatctaactattaaccttgtggtgtgatcttgtcctcttaggtattgtggaatccttgtaggaaatgtcatggcaactcattagtcatcttgaaccaaatgagatgatacatgatggtctcaaattggacatggtggcaccttttgtttcgtgagtggctttcaatttatctccatagaaattgtttaattttcgttattggtgcctgtcaataaaaattacaaacatatgcagagcgttggattcgtggatcgttgaacatcctattgagagacagggccagcctcctaattgtatgacatcgtctctttcacaatatactcattaaatgagaggttgagatttgacttatttgctttatacagcctgcacagtgtggaagacccctctaaagactgaactggggacagcaacaatgggaatctgaccatcctcatgacagtacctggaaagagtctcagaagcttcaagttcagtacgcagcttgaccagtctttcagtcatatagccataggggttgaattagtgtccatcttcccattgtgattaacgttctgatttagcatgcaccttcgaattaagtgaatctattaccatgtgaccaagccattgcattactaatataagcatatcacatttcccttttctccgtgccaactgaatttgaattattttccctcaacttaatcacatgttttcctcacggccaaaagtactctcagtgttacatgattaccacaacaaatgatttaaactttgaacttttgaagttatcgagcaacatggcaaatcctggtcctatatgacataacatgagttcctctgcctattgtaaaattaggaaacacaaaaccaaaatgattatatctggtattatagtgtggtgtataacatatactcacacaagatatgctcttaagatgataaatgtctaatcttccaagtcccaattttgaaaacgttgatattaatttcccctcaaccccactagcctcaaattaaattagcagccttagtgtgaaattaaaagatagctaatgaattgcatttcagactttcacctccccactcacgtagctataactccttaccgtttcaaatctcttcacttccccaattttgttgtgtataaaaacctcttctccacttcactctttccaccacaaactttctaaaactaatcaaca

SEQ ID NO: 72 - protein, 311 aa, unidentified
MASSNRHWPSMFKSKPCNQWQHDINSPLICQKPPFTAEERSPEPKPRWNPKPEQIRILEAIFNSGMVNPPREEIRRIRAQLQEYGQVGDANVFYWFQNRKSRSKHKHKQLHQSSAKPATPSPPTVPNQNYQPTPQSSQTPNSSSSSSEKSEASPVQLGSIKPGATVNVMEGLNAANSPTCSVNQVAYLGSQPEPSPLFFQTESGCEMSAFSELANMLQQQEKMKMGHIAMNDILNGVGEGTANSNGCSGGGGRVTVFINEMAFEVGAGGRVNVREAFGEAMLIHSSGHPVPTNEWGFTLQPLQHGHFYYLV

SEQ ID NO: 73 - protein, 67 aa, unidentified
PEPKPRWNPKPEQIRILEAIFNSGMVNPPREEIRRIRAQLQEYGQVGDANVFYWFQNRKSRSKHKHK

SEQ ID NO: 74 - protein, 388 aa, Glycine max
MSSSNRHWPNMFKSKPCNNPHNQWQHDINSSIVSTGGYQRSPYASGGEERTPEPKPRWNPKPEQIRILEAIFNSGMVNPPRDEIRKIRVQLQEYGQVGDANVFYWFQNRKSRSKHKLRHFQNSMNQNHNAEAQQQQKVDASSLSQTTPPSSSSSSSDKSSSKELAYPIGFSFGFSNVNDVAVPNSPAASVNQTYFQPHNHIDNNLLPQATEPFSFTMHNNNVQGVVDKNTITTLGFSVPQFSSNMMQSQLQCQQNVGPCTSLLLNEIMNYGTLSKKDQDEDKALKITHPQLSFPLTSTPPTTTIAPSISTVPCPITQLQGVGEVAGDRAKCTVFINGVEFEVVMGPFNVHQAFGDEAVLIHSSGNPVPTDKRGITLHPLHHGAYYYLV

SEQ ID NO: 75 - protein, 391 aa, Glycine max
MSSSNRHWPSMFKSKPCNNPHNQWHHDINTSIVSTGCQRSPYANSGGDERTPEPKPRWNPKPEQIRILEAIFNSGMVNPPRDEIRKIRVQLQEYGQVGDANVFYWFQNRKSRSKHKLRHFQNTKNQNNAEAQQQHRVDASSSLSQTTPLSSSSSSSDKSSSKELAYNPNGFSFGFSNVNDVAVPNSPTASVNQTYFHPHNHSDNNLLPQEPFSFTMHNNNGQGFVDNNTITTLGFSVPQFSSNMMQSQLQCQQNVGPCTSLLLSEIMSHGTFSKKDQDQDKALKIMHPQLSNFPLTSTPTTIIAPPISTVLDPSPITQLEGVGEVAAGDRAKCITVFINDVVFEIVMGPFNVRQAFGDEAVLIHSSGNPVPTDEWGITLHPLHHGACYYLV

SEQ ID NO: 76 - protein, 358 aa, Solanum sp.
MASSNRHWPSMFKSKPCNSHHHQWQHDINSSIIQQRPPCNPEERSPEPKPRWNPRPEQIRILEAIFNSGMVNPPRDEIRKIRAKLQEYGQVGDANVFYWFQNRKSRSKHKQRHLQAKAQQQHHNNNNNSSHQPIITSSSSSSDKSSPNSLTFSIGTSNVMDLLNSPTSSVNQQNYNEFLSNEQPFFFTVQPPPVVPTHDHSAGFCFQDSSTFTPHSSSSGLLLNEWMGGISTQAPNNSKKDENDKINLQSQLMSYTVTSTVSPLATTTIPTISHIQGVTVDPNDAGPTRSTVFINDVAFEVGIGPFNVREVFGEDAVLIHSSGEPLITNEWGITIQPLQHGAFYYLLRTSSIASTHHI

SEQ ID NO: 77 - protein, 515 aa, Zea mays; identical to SEQ ID NO: 6

SEQ ID NO: 78 - protein, 306 aa, Oryza sativa
MMALGVPPPPSRAYVSGPLRDDDTFGGDRVRRRRRWLKEQCPAIIVHGGGRRGGVGHRALAAGVSKMRLPALNAATHRIPSTSPLSIPQTLTITRDPPYPMLPRSHGHRTGGGGFSLKSSPFSSVGEERVPDPKPRRNPRPEQIRILEAIFNSGMVNPPRDEIPRIRMQLQEYGQVGDANVFYWFQNRKSRSKNKLRSGGTGRAGLGLGGNRASEPPAAATAHREAVAPSFTPPPILPPQPVQPQQQLVSPVAAPTSLSSSSSDRSSGSSKPARATLTQAMSVTAAMDLLSPLRRSARPRQEQRHV

SEQ ID NO: 79 - protein, 506 aa, Zea mays; identical to SEQ ID NO: 5

SEQ ID NO: 80 - DNA, 278 bp, synthetic construct
  source 1..278: mol_type = other DNA
ccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattgatcccccctcgacag

SEQ ID NO: 81 - DNA, 24 bp, synthetic construct
  source 1..24: mol_type = other DNA
atggaatgatggatgaatgttcac

SEQ ID NO: 82 - DNA, 23 bp, synthetic construct
  source 1..23: mol_type = other DNA
ggtttttctttctagctttgctg

SEQ ID NO: 83 - DNA, 24 bp, synthetic construct
  source 1..24: mol_type = other DNA
gttgccttatgatttcttcggttt

SEQ ID NO: 84 - DNA, 26 bp, synthetic construct
  source 1..26: mol_type = other DNA
ttctcaacagattgataaggtcgaaa

SEQ ID NO: 85 - DNA, 26 bp, synthetic construct
  source 1..26: mol_type = other DNA
ctgcagaaatgttcctcgctgacgta

SEQ ID NO: 86 - DNA, 30 bp, synthetic construct
  source 1..30: mol_type = other DNA
gtcgactattctttctttttggggtttttg

SEQ ID NO: 87 - DNA, 21 bp, synthetic construct
  source 1..21: mol_type = other DNA
atggcgtcgtcgaacaggcac

SEQ ID NO: 88 - DNA, 19 bp, synthetic construct
  source 1..19: mol_type = other DNA
gtccatggccgtcgtcacg

SEQ ID NO: 89 - DNA, 19 bp, synthetic construct
  source 1..19: mol_type = other DNA
cgtgacgacggccatggac

SEQ ID NO: 90 - DNA, 19 bp, synthetic construct
  source 1..19: mol_type = other DNA
cagtcaggctagcggtggc

SEQ ID NO: 91 - DNA, 19 bp, synthetic construct
  source 1..19: mol_type = other DNA
gccaccgctagcctgactg

SEQ ID NO: 92 - DNA, 24 bp, synthetic construct
  source 1..24: mol_type = other DNA
ttacatgacaacaatgtagacggc

(19) United States
(12) Patent Application Publication
(10) Pub. No.: US 2026/0125696 A1
(43) Pub. Date: May 7, 2026

(54) EFFICIENT INDUCTION OF PARTHENOGENESIS IN CROP PLANTS

(71) Applicant: The Regents of the University of California, Oakland, CA (US)

(72) Inventors: Imtiyaz KHANDAY, Davis, CA (US); Hui REN, Davis, CA (US); Venkatesan SUNDARESAN, Davis, CA (US)

(21) Appl. No.: 19/117,511

(22) PCT Filed: Sep. 29, 2023
(86) PCT No.: PCT/US2023/034142

§ 371 Date: Apr. 1, 2025

Related U.S. Application Data
(60) Provisional application No. 63/412,666, filed on Oct. 3, 2022.

(51) Int. Cl. C12N 15/82; C07K 14/415

(52) U.S. Cl. CPC C12N 15/8234; C07K 14/415; C12N 15/8262

(57) ABSTRACT

Methods for improving parthenogenesis efficiency by DWT1 and BABY BOOM transcription factors in plants are provided. A rice embryo trigger transcription factor BABY BOOM1 can initiate embryogenesis when expressed in the unfertilized egg cell through a process called parthenogenesis (Khanday et al., 2019. Nature 565: 91-95). The parthenogenesis efficiency by BABY BBOM1 itself is 10-29%. This invention describes methods of high frequency of parthenogenesis by simultaneous expression of BABY BOOM and DWT1 transcription factors. When BABY BOOM1 and DWT1 are expressed together through egg cell-specific promoters, parthenogenesis efficiencies of up to 90% are achieved. These high parthenogenesis efficiencies are a prerequisite for field applications of synthetic apomixis in crop plants.

### CROSS-REFERENCE TO RELATED PATENT APPLICATIONS

The present patent application is a US National Phase Application Under 371 of International Application PCT/US2023/034142 filed Sep. 29, 2023, which claims benefit of priority to U.S. Provisional Patent Application No. 63/412,666, filed Oct. 3, 2022, which are incorporated by reference for all purposes.

### REFERENCE TO A SEQUENCE LISTING

The instant application contains a Sequence Listing which has been submitted electronically in XML format and is hereby incorporated by reference in its entirety. Said XML copy, created on Nov. 21, 2023, is named 081906-1409451-251010PC_SL.xml and is 147,326 bytes in size.

### BACKGROUND OF THE INVENTION

Fusion of haploid gametes—an egg and a sperm during fertilization initiates embryogenesis in sexually reproducing plants. The molecular basis of embryo initiation after fertilization is still obscure. Previous transcriptome analysis of rice gametes and zygotes identified two transcription factors OsBBM1 and OsDWT1, belonging to the PLETHORA/BABY BOOM clade of APETALA2-family and WUSCHEL-Homebox or WOX family, respectively that are only expressed from the male alleles in the zygote after fertilization (Anderson et al., Developmental Cell 2017, 43:349-358.e4 (2017)). It was further shown that OsBBM1 functions to initiate embryogenesis after fertilization in rice plants. In transgenic rice with OsBBM1 expressed under an egg cell-specific promoter, the result is parthenogenesis (embryo development without fertilization) and the production of haploid progeny (Khanday et al., Nature 565:91-95 (2019)). The parthenogenesis frequencies arising from ectopic expression of OsBBM1 in the egg cell were found to be in the range of 10-29% (Khanday et al., Nature 565:91-95 (2019)).

### BRIEF SUMMARY OF THE INVENTION

In some embodiments, a plant is provided comprising: a first expression cassette comprising a first plant egg-specific promoter operably linked to a polynucleotide encoding a Dwarf Tiller 1 (DWT1) polypeptide; and a second expression cassette comprising a second plant egg-specific promoter operably linked to polynucleotide encoding a Baby boom polypeptide, wherein the plant has more efficient parthenogenesis than a control plant lacking at least the first, and optionally the second, expression cassette.

In some embodiments, the plant is diploid and progeny from the plant resulting from parthenogenesis are haploid.

In some embodiments, the plant further comprises sufficient mitosis instead of meiosis (MiME) expression cassettes comprising a promoter operably linked to gRNAs to induce a MiME phenotype such that the plant produces clonal seed. In some embodiments, the MiMe expression cassettes comprise: an expression cassette comprising a promoter operably linked to a gRNA that targets OSD1 or an ortholog thereof: an expression cassette comprising a promoter operably linked to a gRNA that targets ATREC8 or an ortholog thereof: an expression cassette comprising a promoter operably linked to a gRNA that targets SPO11, or PRD1, or PRD2 or PRD3/PAIR1 or an ortholog thereof.

In some embodiments, the DWT1 polypeptide comprises an amino acid sequence at least 80%, 85%, 90%, 95%, or 98% identical to SEQ ID NO:33. In some embodiments, the DWT1 polypeptide comprises an amino acid sequence at least 65%, 70%, 75%, 80%, 85%, 90%, 95%, or 98% identical to SEQ ID NO: 1, 2, 3, 4, 5, 6 or 72.

In some embodiments, the Baby boom polypeptide comprises an amino acid sequence at least 80%, 85%, 90%, 95%, or 98% identical to SEQ ID NO:10-29.

In some embodiments, the first egg-specific promoter and the second egg-specific promoter are the same. In some embodiments, the first egg-specific promoter and the second egg-specific promoter are the different. In some embodiments, the first egg-specific promoter and the second egg-specific promoter or both comprise SEQ ID NO:30, SEQ ID NO: 31 or SEQ ID NO:32.

In some embodiments, the plant is a rice plant.

Also provided is a method of making the plant as described above or elsewhere herein. In some embodiments, the method comprises, introducing the first expression cassette and the second expression cassette into the plant. In some embodiments, the method further comprises selecting plant cells, tissues, or plants with the introduced expression cassettes, and optionally regenerating plants from the selected plant cells, tissues, or plants.

In some embodiments, the introducing comprises transformation of the plant with the first or second or both expression cassettes, introducing the first or second or both expression cassettes into the plant with a sexual cross, or introducing one of the first and second expression cassettes into the plant via transformation and introducing one of the first and second expression cassettes into the plant via a sexual cross.

Also provided is a method of generating haploid progeny (or progeny having half the ploidy of the parent plant(s)). In some embodiments, the method comprises cultivating a plant as described above or elsewhere herein (e.g., but not having the MiMe phenotype); and collecting haploid (or progeny having half the ploidy of the parent plant(s)) seed from the plant.

Also provided is a method of generating clonal progeny. In some embodiments, the method comprises growing a plant as described above or elsewhere herein and having the MiMe phenotype, and collecting clonal seed from the plant.

Also provided is a nucleic acid comprising an expression cassette comprising a plant egg-specific promoter operably linked to a polynucleotide encoding a DWT1 polypeptide. In some embodiments, the promoter comprises SEQ ID NO:30, SEQ ID NO:31 or SEQ ID NO:32. In some embodiments, the DWT1 polypeptide comprises an amino acid sequence at least 80%, 85%, 90%, 95%, or 98% identical to SEQ ID NO:33. In some embodiments, the DWT1 polypeptide comprises an amino acid sequence at least 65%, 70%. 75%, 80%, 85%, 90%, 95%, or 98% identical to SEQ ID NO: 1, 2, 3, 4, 5, 6 or 72.

### Definitions

An “endogenous” or “native” gene or protein sequence, as used with reference to an organism, refers to a gene or protein sequence that is naturally occurring in the genome of the organism.

A polynucleotide or polypeptide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence, e.g., from a different gene in the same species, or an allele from a different ecotype or variety).

The term “promoter,” as used herein, refers to a polynucleotide sequence capable of driving transcription of a coding sequence in a cell. Thus, promoters can include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. It includes plants of a variety of ploidy levels, including aneuploid, polyploid, diploid, haploid, and hemizygous.

A “transgene” is used as the term is understood in the art and refers to a heterologous nucleic acid introduced into a cell by human molecular manipulation of the cell's genome (e.g., by molecular transformation). Thus, a “transgenic plant” is a plant that carries a transgene, i.e., is a genetically-modified plant. The transgenic plant can be the initial plant into which the transgene was introduced as well as progeny thereof whose genomes contain the transgene.

The phrase “nucleic acid” or “polynucleotide sequence” refers to a single or double-stranded polymer of deoxyribonucleotide or ribonucleotide bases read from the 5′ to the 3′ end. Nucleic acids may also include modified nucleotides that permit correct read through by a polymerase, and/or formation of double-stranded duplexes, and do not significantly alter expression of a polypeptide encoded by that nucleic acid.

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full-length nucleic acid sequences as well as non-full length sequences derived from the full length sequences. It should be further understood that the sequence includes the degenerate codons of the native sequence or sequences which may be introduced to provide codon preference in a specific host cell.

The terms “identical” or percent “identity.” in the context of two or more nucleic acids or polypeptide sequences, refer to two or more sequences or subsequences that are the same or have a specified percentage of nucleotides or amino acid residues that are the same, when compared and aligned for maximum correspondence over a comparison window, as measured using one of the following sequence comparison algorithms or by manual alignment and visual inspection. Two nucleic acid sequences or polypeptides are said to be “identical” if the sequence of nucleotides or amino acid residues, respectively, in the two sequences is the same when aligned for maximum correspondence as described below. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, California, USA).

The phrase “substantially identical,” used in the context of two nucleic acids or polypeptides, refers to a sequence that has at least 50% sequence identity with a reference sequence (e.g., any of SEQ ID NOs: 1-69). Alternatively, percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein; preferably BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction is halted when: the cumulative alignment score falls off by the quantity X from its maximum achieved value: the cumulative score goes to zero or below; due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see. e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

An “expression cassette” refers to a nucleic acid construct that, when introduced into a host cell, results in transcription and/or translation of an RNA or polypeptide, respectively.

### BRIEF DESCRIPTION OF THE DRAWINGS

FIG. 1A-B: Schematic drawings of T-DNA vectors used for inducing parthenogenesis in Rice. FIG. 1A: pEC1.2::OsDWT1 transgene in a binary vector (pCAMBIA2300) for egg cell expression of OsDWT1. FIG. 1B: pEC1.2::OsBBM1 in a binary vector (pCAMBIA1300) for egg cell expression of OsBBM1.

FIG. 2A-B: Characterization of parthenogenetic haploids. FIG. 2A. Left, a haploid plant from transgenic line #12b from T2 generation. Right, T2 diploid progeny sibling plant from the same line #12b. Haploids are dwarf with narrow leaves and sterile due to meiotic defects. FIG. 2B. Left, a haploid panicle showing complete sterility due to meiotic defects. Right, a fertile control diploid panicle from the same line #12b.

FIG. 3A-C: Flow-cytometric DNA histograms for ploidy determination. FIG. 3A. Sorted nuclei from leaves of a diploid plant showing a 2n peak at 160. FIG. 3B. Parthenogenetic haploid showing a 1n peak at 80. FIG. 3C. A mixed sample of haploid and diploid nuclei showing 1n and 2n peaks.

### DETAILED DESCRIPTION OF THE INVENTION

The inventors have discovered that expressing a Dwarf Tiller 1 (DWT1) polypeptide and Baby boom polypeptide in the egg of a plant greatly improves efficiency of parthenogenesis from the resulting plant compared to expression of a Baby boom polypeptide alone. Expression of DWT1 with Baby boom in egg cells (i.e., plant egg cells) allows for agronomically useful levels of parthenogenesis.

Thus in some embodiments, targeting expression of BABYBOOM and DWT1 to egg cells of a plant will result in production of progeny that have half the number of chromosomes compared to the parent. In addition, in embodiments in which the Meiosis-to-mitosis (MiMe) phenotype has been induced, synthetic apomixis will be induced in the plant at high efficiencies, resulting in clonal seed production.

Accordingly, the disclosure provides for egg-expression of DWT1 and Baby boom polypeptides in plants and optionally further genetic manipulation to result in the Mime phenotype.

DWT1 polypeptides are homeobox transcription factors naturally-expressed in plant reproductive structures (see, e.g., Wang W, et al. (2014) PLOS Genet 10 (3): e1004154; Fang et al, (2020). New Phytologist 225:1234-1246: Anderson et al., 2017. Developmental Cell 43:349-358) and are characterized by a highly conserved (>85% identity) portion of 67 amino acids (SEQ ID NO:33). See, for example, the following alignment with the 67 amino acid portion bolded:

| | |
|---|---|
| SoybeanDWT1 | ------------------------------------------------------------ 0 |
| SoybeanDWL1 | ------------------------------------------------------------ 0 |
| AmborellaDWT1 | ------------------------------------------------------------ 0 |
| TomatoDWT1 | ------------------------------------------------------------ 0 |
| MaizeDWT1 | ---------------------------------------------------------MAS 3 |
| MaizeDWL1 | ----------------------------------------------------------MA 2 |
| OsDWT1 | ------------------------------------------------------------ 0 |
| OsDWL1 | MMALGVPPPPSRAYVSGPLRDDDTFGGDRVRRRRRWLKEQCPAIIVHGGGRRGGVGHRAL 60 |
| OsDWL2 | ------------------------------------------------------------ 0 |
| MaizeDWL2 | ------------------------------------------------------------ 0 |
| SoybeanDWT1 | -MSSSNRHWPNMFKSKPCNNPHNQWQHDINSSIVSTGG---------------------- 37 |
| SoybeanDWL1 | -MSSSNRHWPSMFKSKPCNNPHNQWHHDINTSIVSTGC---------------------- 37 |
| AmborellaDWT1 | -MASSNRHWPSMFKSKPCN----QWQHDINSPLICQ------------------------ 31 |
| TomatoDWT1 | -MASSNRHWPSMEKSKPCNSHHHQWQHDINSSIIQQ------------------------ 35 |
| MaizeDWT1 | SFNNKTSHWPSMFRSKHAA---EPWQ---AQPDISSS----PPSLLSGGGSSSTTTIGRC 53 |
| MaizeDWL1 | SSSFNNSHWPSMFRSKHAA---EPCQ--TTQPDISSS----PESLLSAGGASTTTTTGRC 53 |
| OsDWT1 | -MASSNRHWPSMERSKHA----TQPW---QTQPDMAGS---PPSLLSGSSAGSAGGGGYS 49 |
| OsDWL1 | AAGVSKMRLPALNAATHRIPSTSPLSIPQTLTITRDP----PYPMLPRSHGHRTGGGGFS 116 |
| OsDWL2 | -MASPNRHWPSMFRSNLACN----IQQQQ-QPDMNGNGSSSSSFLLSPPTAATTGNGKPS 54 |
| MaizeDWL2 | -MASSNRHWPSMYRSSLACN----FQQPQPQPDMNNG-------------------GKSS 36 |
| | . : * : :. |
| SoybeanDWT1 | YQRSP-YASGGEERTPEPKPRWNPKPEQIRILEAIFNSGMVNPPRDEIRKIRVQLQEYGQ 96 |
| SoybeanDWL1 | QRSPY-ANSGGDERTPEPKPRWNPKPEQIRILEAIFNSGMVNPPRDEIRKIRVQLQEYGQ 96 |
| AmborellaDWT1 | --KPP---FTAEERSPEPKPRWNPKPEQIRILEAIFNSGMVNPPREEIRRIRAQLQEYGQ 86 |
| TomatoDWT1 | --RPP---CNPEERSPEPKPRWNPRPEQIRILEAIFNSGMVNPPRDEIRKIRAKLQEYGQ 90 |
| MaizeDWT1 | LKHPLSGYSGGEERTPDPKPRWNPRPEQIRILEAIFNSGMVNPPRDEIPRIRMRLQEYGQ 113 |
| MaizeDWL1 | LKHSIS--VGGEERAPDPKPRWNPRPEQIRILEAIFNSGMVNPPRDEIPRIRMRLQQYGQ 111 |
| OSDWT1 | LKSSPF-SSVGEERVPDPKPRWNPRPEQIRILEAIFNSGMVNPPRDEIPRIRMQLQEYGQ 108 |
| OsDWL1 | LKSSPF-SSVGEERVPDPKPRRNPRPEQIRILEAIFNSGMVNPPRDEIPRIRMQLQEYGQ 175 |
| OSDWL2 | LLSSGC--E-EGTRNPEPKPRWNPRPEQIRILEGIFNSGMVNPPRDEIRRIRLQLQEYGQ 111 |
| MaizeDWL2 | LMSSRC--EENGGRNPEPRPRWNPRPEQIRILEGIFNSGMVNPPRDEIRRIRLQLQEYGP 94 |
| | * *:*:** **:*******.**.*********:** :** :**:** |
| SoybeanDWT1 | VGDANVFYWFQNRKSRSKHKLRHFQNSMNQNH------------------------NAEA 132 |
| SoybeanDWL1 | VGDANVFYWEQNRKSRSKHKLRHFQNTKNQNN------------------------AEAQ 132 |
| AmborellaDWT1 | VGDANVFYWFQNRKSRSKHKHKQLHQSSAKPA------------------------TPSP 122 |
| TomatoDWT1 | VGDANVFYWFQNRKSRSKHKQRHLQAKAQQQH------------------------HNNN 126 |
| MaizeDWT1 | VGDANVFYWFQNRKSRSKNKQRIGQLGL------GLARAPGCG-------------AAAP 154 |
| MaizeDWL1 | VGDANVFYWFQNRKSRSKNELR STAGTGRLGLQGLARAPGRGA-----A------AAPP 160 |
| OsDWT1 | VGDANVFYWFQNRKSRSKNKLRSGGIGRAGLGLGGNRASAPA---AAHREAVAPSFTPPP 165 |
| OsDWL1 | VGDANVFYWFQNRKSRSKNKLRSGGTGRAGLGLGGNRASEPPAAATAHREAVAPSETPPP 235 |
| OsDWL2 | VGDANVPYWFQNRKSRTKNKLRAAGHHHHHGR----AAALPRASAPPSTNIVLPSAAAAA 167 |
| MaizeDWL2 | VGDANVFYWFQNRKSRTKHKLRAAGQLQPSGS----GRSALQA-----------RACAPA 139 |
| | ****************:*:* : |
| SoybeanDWT1 | QQQ------QKVDASSLSQTTPPSSSSSSSDKSSSKELAY-PIGF--------------- 170 |
| SoybeanDWL1 | QQH------RVDASSSLSQTTPLSSSSSSSDKSSSKELAYNPNGF--------------- 171 |
| AmborellaDWT1 | PTVPNQ---NYQPTPQSSQTPNSSSSSSEKSEASPVQLGSIKP----------------- 162 |
| TomatoDWT1 | N--------------NSSHQPIITSSSSSSDKSSPNSLTF--S----------------- 153 |
| MaizeDWT1 | PVTPQPLIQNQFQVLASPAQAPASSSSSSSDRSSGSSKPAPQP-M--------------- 198 |
| MaizeDWL1 | PVEPPPLVQNQFHMLASPAQAPTSSSSSSSDRSSGSSKPAABPAM--------------- 205 |
| OsDWT1 | PILPAPQPVQPQQQLVSPVAAPTSSSSSSSDRSSGSSKPARATST--------------- 210 |
| OsDWLT1 | L--PPQPVQPQQQLVSPVAAPTSLSSSSSDRSSGSSKPARATLT---------------- 278 |
| OsDWL2 | PLTPPR---RHLLA------ATSSSSSSSDRSSGSSKSV----KPA-------------- 200 |
| MaizeDWL2 | PVTPPR---NLQLAAAAPVAPPTSSSSSSSDRSSGSSSSKSVTVTPTTAVALASPAGAAP 196 |
| | : ***....:* . |
| SoybeanDWT1 | ----SFGFSN-VNDVAVPNSPAAS-----VNQTYFQEHNHIDNNLLPQ---------ATE 211 |
| SoybeanDWL1 | ----SFGFSN-VNDVAVPNSPTAS-----VNQTYFHPHNHSDNNLLPQ-----------E 210 |
| AmborellaDWT1 | -----GATVNVMEGLNAANSPTCS-----VNQVAYLGSQPEP-----------------S 195 |
| TomatoDWT1 | -----IGTSNV---MQLINSPISS-----VNQQNYNEFLSNE-----------------Q 183 |
| MaizeDWT1 | ------S--ATAAAMNFPGPLGAA-----CAQMYYQAHPVAPVSALP-AHKVQDPVASDE 244 |
| MaizeDWL1 | ------P--ATAAPMDLLGPLAAA-----CPQMYYQGSPVAP------AHKVLDLVASVE 246 |
| OsDWT1 | -----QAMSVITAMDLLSPLAAA------CHQQMLYQGQPLESPPAP-APKVHGIVPHDE 256 |
| OsDWL1 | -----QAMSVTAAMDLISPLRRS------ARPRQBQRHV--------------------- 306 |
| OsDWL2 | ----AAALLTSAAIDLESPAPAPTTQLPACQLYYHSHPTPLARD------DQLITSPESS 250 |
| MaizeDWL2 | AAVFRQQGVMPTTAMDLLTPLESSSAALAARQLYYQYHSQIMAPAAPPMEDTVIASPE-- 254 |
| | : |
| SoybeanDWT1 | PFSFTMHNNNVQG----VVDKNTI-------------------------TTLGFSVPQFS 242 |
| SoybeanDWL1 | PFSFTMHNNNGQG----FVDNNTI-------------------------TTLGFSVPQFS 241 |
| AmborellaDWT1 | PLFFQTESGCEMS----AFS-----------------------------ELA-------- 214 |
| TomatoDWT1 | PFFFTVQPPPVVP----THD-----------------------------HSAGFCFQDSS 210 |
| MaizeDWT1 | PVFQEWLQ----GYELSAAEV-ASILGGQYRHD--VPVQQQPPATLPAGAFLGLYNEV-- 295 |
| MaizeDWL1 | PVFQPWPQ----GYCLSAAEV-ATILGGQYMH---VPVQQQPPAPLPAGALLGLCNDV-- 296 |
| OsDWT1 | PVFLQWPQ----SPCLSAVDLGAAILGGQYMHL-PVPAPQPPSSPGAAGMFWGLQNDV-- 311 |
| OsDWL1 | ------------------------------------------------------------ 306 |
| OsDWL2 | SLLLQWPA----SQYMPATELGGV-LGSSS-HTQTPAAITTHPSTISPSVLLGLCNEA-- 302 |
| MaizeDWL2 | QFLPQWQQGGQQHYYLPATELGGV-LDGHSHHTHEPPAAIHRPVSLSPSVLFGLQNEA-- 311 |
| SoybeanDWT1 | SNMMQSQLQCQQNV----------------GPCTSLLENEIMNYGTL---SKKDQDEDKA 283 |
| SoybeanDWL1 | SNMMQSQLQCQQNV----------------GPCTSLLLSEIMSHGTF---SKKDQDQDKA 282 |
| AmborellaDWT1 | ----------------------------------NML------------------QQQEK 222 |
| TomatoDWT1 | T---------FTPH----------------SSSSGLLINEWMGGISTQAPNNSKKDENDK 245 |
| MaizeDWT1 | ----TEPT--VTGHRTCAWGPAGLGQFWPVGGADHHQHHKHNIT-AATNTVARDAAHEHA 348 |
| MaizeDWL1 | ----TEPTAVVTGHKTCAWGPAGLGQSWPCGGADHHQPGKNNNT-AARELA----HEDDA 347 |
| OsDWT1 | ----QAPN--NTGHKSCAWS-AGLGQHW-CGSADQLGLGKSSAASIATVSRPEEAHDVDA 363 |
| OsDWL1 | ------------------------------------------------------------ 306 |
| OsDWL2 | ----LGQHQQETMDDMMITCSNPSKV-FD-----HHSMQDMSCT----DAVSAVNRDDEK 348 |
| MaizeDWL2 | ----LRQDYCADISVVPTKGLGHGHQFWNSTTCGSDMGNSNSKI----DAVSAVIRDDEK 363 |
| SoybeanDWT1 | LKITHPQLSFPLTSTPPTT---------------------------------TIAPSIS- 309 |
| SoybeanDWL1 | LKIMHPQLSNFPLTSTPTT---------------------------------IIAPPIS- 308 |
| AmborellaDWT1 | MKMGH---------------------------------------------------IAM- 230 |
| TomatoDWT1 | INLQSQLMSYTVIST--------------------------------------VSPLAT- 266 |
| MaizeDWT1 | TTLGLLQYGFEASAAMETASA-------A--VPLAASPGTA------AS--VATAGLT-- 389 |
| MaizeDWL1 | TKLGLLQYGFGATTAMEAAPA-------V--APLAASPAGGAVIMASVS--ASTAGLT-- 394 |
| OsDWT1 | TKHGLLQYGFGITTPQVHVDVISSAAGVLPPVPSSPSPPNAAVTVASV---AATASLT-- 418 |
| OsDWL1 | ------------------------------------------------------------ 306 |
| OsDWL2 | ARLGLLHYGIGVTAAANPAPHHHHHHHHL------ASPVHDAVSAADASTAAMILPETIT 402 |
| MaizeDWL2 | SRLGLLHYYGLAGATTTAAA-----------------AV-------------APAPLAAD 393 |
| SoybeanDWT1 | -------TV---PCPITQLQGVGEVA-GD---------------------------RAKC 331 |
| SoybeanDWL1 | -------TVL-DPSPITQLEGVGEVAAGD---------------------------RAKC 333 |
| AmborellaDWT1 | -------ND---ILNGVGEGTANSNGCSG---------------------------GGGR 253 |
| TomatoDWT1 | -------TT---IPTISHICGVTVDENDA---------------------------GPTR 289 |
| MaizeDWT1 | --SLPASTNA-VVVNYDLLQGLAVPGSGSGAVGVSTGGAPPVAVAAAPTAAQEGVVVALC 446 |
| MaizeDWL1 | --GFPASTNG-VVANYDLLQGLAVPGGGAGAGRAPA--AVAVAADAAPTAAQEG-VVALC 448 |
| OsDWT1 | --DFAASAISAGAVANNQFQGLADFGLVAGACS----GAGAAAAAAAP---EAGSSVAAV 469 |
| OsDWL1 | ------------------------------------------------------------ 306 |
| OsDWL2 | AAATPSNVVATSSALADQLQGLLDAGLLQGGAAPPPPSATVVAVSRD--------DETMC 454 |
| MaizeDWL2 | -AAAGTATLLPSSAASDQLQGLLDAAGLLMGETPPTPTATVVAVARD--------AVTCA 444 |
| SoybeanDWT1 | -TVFI-NGVEFEVVMG-PFNVHQAFGDEAVLIHSSGN-------PVPTDKRGITLHPLHH 381 |
| SoybeanDWL1 | ITVFI-NDVVFEIVMG-PENVRQAFGDEAVLIHSSGN-------PVPTDEWGITLHPLHH 384 |
| AmborellaDWT1 | VTVFI-NEMAFEVGAGGRVNVREAFGE-AMLIHSSGH-------PVPTNEWGFTLQPLQH 304 |
| TomatoDWT1 | STVFI-NDVAFEVGIG-PFNVREVFGEDAVLIHSSGE-------PLITNEWGITIQPLQH 340 |
| MaizeDWT1 | ITDSVTGKSVAHNVAAARLDVRAQFGEAAVLLRAVGDRGGLDLVPVPVDALGCTVEPLQH 506 |
| MaizeDWL1 | ITDSITGKSVAHNVAAARLQVRAQFGEAAVLLRCGGE-RGLDLEPVPVDASGCTVEPLQR 507 |
| OsDWT1 | VCVSVAGAAPPLEYPAAHENVR-HYGDEAELLRY---RGGSRTEPVPVDESGVTVEPLQQ 525 |
| OsDWL1 | ------------------------------------------------------------ 306 |
| OsDWL2 | -----TKITSYSEPATMHLNVK-MFGEAAVIVRYSGE-------PVINDDSGVIVEPLQQ 501 |
| MaizeDWL2 | ----ATATAQFSVPASMRLDVRLAFGEAALLARHTGE-------AVPVDESGVTVEPLQQ 493 |
| SoybeanDWT1 | GAYYYLV----------- 388 (SEQ ID NO: 74) |
| SoybeanDWL1 | GACYYLV----------- 391 (SEQ ID NO: 75) |
| AmborellaDWT1 | GHEYYLV----------- 311 (SEQ ID NO: 72) |
| TomatoDWT1 | GAFYYLLRTSSIASTHHI 358 (SEQ ID NO: 76) |
| MaizeDWT1 | GAFYYVLV---------- 514 (SEQ ID NO: 4) |
| MaizeDWL1 | GAFYYVLL---------- 515 (SEQ ID NO: 77) |
| OsDWT1 | GAVYIVVM*--------- 533 (SEQ ID NO: 1) |
| OsDWL1 | ------------------ 306 (SEQ ID NO: 78) |
| OsDWL2 | GATYYVLVSEEAVH*--- 515 (SEQ ID NO: 2) |
| MaizeDWL2 | DTLYYVLMQATNN----- 506 (SEQ ID NO: 79) |

DWT1 protein sequences include the Amborella trichopoda DWT1 protein sequence (SEQ ID NO:72). Even though Amborella is a basal angiosperm, i.e., its lineage diverged from those of monocots and dicots early in the evolution of flowering plants, the corresponding portion of the Amborella DWT1 protein (SEQ ID NO:73) has 88% identity to rice DWT1 in the highly conserved 67 amino acid domain.

Moreover, the 67 amino acid sequence has comparatively low conservation (˜40%-70% identity) with WUS, WOX2 and other Wuschel-related protein families that are not DWT orthologs. The sequence of the 67 amino acid portion is therefore DWT/DWL-specific.

Accordingly, DWT1 polypeptides can comprise an amino acid sequence at least 80, 85, 90, 95, 98, 99 or 100% identical to PDPKPRWNPRPEQIRILEAIFNSGMVNPPRDEIPRIRMQLQEYGQVGDANVFYWFQNRKSRSKNKLR (SEQ ID NO: 33). In some embodiments, the DWT1 polypeptide is from a species of plant of the genus Abelmoschus, Allium, Apium, Amaranthus, Arachis, Arabidopsis, Asparagus, Atropa, Avena, Benincasa, Beta, Brassica, Cannabis, Capsella, Cica, Cichorium, Citrus, Citrullus, Capsicum, Carthamus, Cocos, Coffea, Cucumis, Cucurbita, Cynasa, Daucus, Diplotaxis, Dioscorea, Elais, Eruca, Foeniculum, Fragaria, Glycine, Gossypium, Helianthus, Heterocallis, Hordeum, Hyoscyamus, Ipomea, Lactuca, Lagenaria, Lepidium, Linum, Lolium, Luffa, Luzula, Lycopersicon, Malus, Manihot, Majorana, Medicago, Momodica, Musa, Nicotiana, Olea, Oryza, Panicum, Pastinaca, Pennisetum, Persea, Petroselinium, Phaseolus, Physalis, Pinus, Pisum, Populus, Pyrus, Prunus, Raphanus, Saccharum, Secale, Senecio, Sesamum, Sinapis, Solanum, Sorghum, Spinacia, Theobroma, Trichosantes, Trigonella, Triticum, Turritis, Valerianelle, Vitis, Vigna, or Zea. Exemplary DWT1 polypeptides can comprise, for example, an amino acid sequence at least 65, 70, 75, 80, 85, 90, 95, 98, 99 or 100% identical to SEQ ID NO: 1, 2, 3, 4, 5, 6 or 72.

Any naturally- or non-naturally-occurring active BABYBOOM polypeptide from a sexually reproducing plant can be expressed as described herein so long as the polypeptide (and/or RNA encoding the polypeptide) is expressed in egg cells in the plant. BABY BOOM polypeptides contain two conserved AP2 domains. The corresponding transcripts lack a miR172 binding site, thereby distinguishing BABY BOOM polypeptides from many other AP2 domain proteins that contain a miR172 binding site. In some embodiments, the BABYBOOM polypeptide is from a species of plant of the genus Abelmoschus, Allium, Apium, Amaranthus, Arachis, Arabidopsis, Asparagus, Atropa, Avena, Benincasa, Beta, Brassica, Cannabis, Capsella, Cica, Cichorium, Citrus, Citrullus, Capsicum, Carthamus, Cocos, Coffea, Cucumis, Cucurbita, Cynasa, Daucus, Diplotaxis, Dioscorea, Elais, Eruca, Foeniculum, Fragaria, Glycine, Gossypium, Helianthus, Heterocallis, Hordeum, Hyoscyamus, Ipomea, Lactuca, Lagenaria, Lepidium, Linum, Lolium, Luffa, Luzula, Lycopersicon, Malus, Manihot, Majorana, Medicago, Momodica, Musa, Nicotiana, Olea, Oryza, Panicum, Pastinaca, Pennisetum, Persea, Petroselinium, Phaseolus, Physalis, Pinus, Pisum, Populus, Pyrus, Prunus, Raphanus, Saccharum, Secale, Senecio, Sesamum, Sinapis, Solanum, Sorghum, Spinacia, Theobroma, Trichosantes, Trigonella, Triticum, Turritis, Valerianelle, Vitis, Vigna, or Zea. In some embodiments the BABYBOOM polypeptide is identical or substantially identical to any of SEQ ID NOs: 10, 11, 12, 13, 14, 15, 16, 17, 18, 19, 20, 21, 22, 23, 24, 25, 26, 27, 28, or 29. See, also, Chahal, et al., Front. Plant Sci., 14 Jul. 2022.

As noted above, both the DWT1 polypeptide and the Baby boom polypeptide will be expressed in the egg cell of the plant. In some embodiments, the plant comprises (i) a heterologous expression cassette comprising a promoter that at least directs expression to egg cells operably linked to a DWT1 polypeptide as described herein and (ii) a heterologous expression cassette comprising a promoter that at least directs expression to egg cells operably linked to a BABYBOOM polypeptide as described herein. In some embodiments, the promoter is egg cell-specific, meaning the promoter drives expression only or primarily in egg cells. “Primarily” means that if there is expression in other tissue the levels are no more than 1/10 of the expression levels in egg cells as measured by quantitative RT-PCR.

Exemplary promoters that drive expression in at least egg cells of a plant include, but are not limited to, the promoter of the egg-cell specific gene EC1.1 (e.g., SEQ ID NO:32), EC1.2, EC1.3, EC1.4, or EC1.5. See, e.g. Sprunck et al. Science, 338:1093-1097 (2012); AT2G21740; Steffen et al., Plant Journal 51:281-292 (2007). In some embodiments, the rice-specific promoter comprises SEQ ID NO:31, i.e., the rice egg cell-specific promoter sequence from the LOC_Os03g18530 OsECA1 gene. In some embodiments, the Arabidopsis DD45 promoter is used to express in rice egg cell (Ohnishi et al. Plant Physiology 165:1533-1543 (2014). An exemplary DD45 promoter sequence can comprise, for example, SEQ ID NO:30. Other promoters that can be used for egg cell expression include promoters of the egg cell-specific ECS1 (SEQ ID NO:69) and ECS2 (SEQ ID NO: 70) genes (Yu et al. 2021, Nature 592:433-437) and the RWD2 gene (Köszegi et al. 2011 The Plant Journal 67:280-291).

Other promoters that are expressed in egg cells, but are not necessarily egg-cell specific, are described in, e.g., Anderson et al., The Plant Journal 76:729-741 (2013). In some embodiments, the expression cassette further comprises a transcriptional terminator. Exemplary terminators can include, but are not limited to, the rbcS E9 or nos terminators. In some embodiments, the expression cassette will include an egg cell enhancer. Exemplary egg cell enhancers include, but are not limited to, the EC1.2 enhancer or EASE enhancer (Yang et al., Plant Physiol. 139:1421-32 (2005). In some embodiments, a different egg-specific promoter is operably linked to the coding sequence for DWT1 and Baby boom to avoid possible recombination events.

In other embodiments, mutations can be introduced into one or both of the native BABYBOOM promoter and the native DWT1 promoter such that BABYBOOM and/or DWT1 is expressed in egg cells based from the modified native promoter. In such embodiments, one or more nucleotide of the BABYBOOM and/or DWT1 promoter is modified by non-natural substitution, deletion or insertion.

Manipulation of the native promoter can be achieved via site-directed or random mutagenesis. Methods for introducing genetic mutations into plant genes and selecting plants with desired traits are well known and can be used to introduce mutations into the BABYBOOM and/or DWT1 promoter to cause the promoter to drive expression in plant egg cells. For instance, seeds or other plant material can be treated with a mutagenic insertional polynucleotide (e.g., transposon, T-DNA, etc.) or chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used. Plants having a mutated BABYBOOM promoter can then be identified, for example, by phenotype or by molecular techniques, including but not limited to TILLING methods. See, e.g., Comai, L. & Henikoff, S. The Plant Journal 45, 684-694 (2006).

Other mutation induction systems, such as genome editing methods, can be used to target mutations in the BABYBOOM and/or DWT1 promoter, having the advantages of increasing the frequency of single and multiple mutations at a defined target site (Lozano-Juste, J., and Cutler, S. R. (2014) Trends in Plant Science 19, 284-287). The sequence-specific introduction of a double stranded DNA break (DSB) in a genome leads to the recruitment of DNA repair factors at the breakage site, which then repair lesion by either the error-prone non-homologous end joining (NHEJ) or homologous recombination (HR) pathways. NHEJ repairs the breaks, but is imprecise and often creates diverse mutations at and around the DSB. In cells in which the HR machinery repairs the DSB, sequences with homology flanking the DSB, including exogenously supplied sequences, can be incorporated at the region of the DSB. DSBs can therefore be leveraged by geneticists to increase the frequency of mutations at defined sites, however intrinsic differences between the relative roles of HR and NHEJ can affect the mutation types at a targets locus. A number of technologies have been developed to create DSBs at specific sites including synthetic zinc finger nucleases (ZFNs), transcription activator-like endonucleases (TALENs) and most recently the clustered regularly interspaced short palindromic repeats (CRISPR)/CRISPR-associated protein 9 (Cas9) system. This system is based on a bacterial immune system against invading bacteriophages in which a complex of 2 small RNAs, the CRISPR-RNA (crRNA) and the trans-activating crRNA (tracrRNA) directs a nuclease (Cas9) to a specific DNA sequence complementary to the crRNA. Using any of these systems, one can create DSBs at pre-determined sites in cells expressing the genome editing constructs. In order for homologous recombination to occur, a DNA cassette homologous to the targeted site must be provided, preferably at a high concentration so that HR is favored or NHEJ.

The present disclosure also provides for nucleic acids, including isolated nucleic acids, nucleic acid expression cassettes, and expression vectors, that encode a heterologous egg cell-expressing promoter operably linked to a DWT1 polypeptide coding sequence, and optionally further comprising an expression cassette comprising a heterologous egg cell-expressing promoter operably linked to a Baby boom polypeptide coding sequence as described herein. Also provided are host cells comprising the nucleic acids.

In some embodiments, recombinant DNA vectors suitable for transformation of plant cells and comprising the expression cassette are prepared. Techniques for transforming a wide variety of higher plant species are well known and described in the technical and scientific literature. See, e.g., Weising et al. Ann. Rev. Genet. 22:421-477 (1988). In some embodiments, the vector comprising the sequences (e.g., promoters or CENH3 coding regions) comprises a marker gene that confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or Basta.

In some embodiments, any of a variety of different expression constructs, such as expression cassettes and vectors suitable for transformation of plant cells, can be prepared. Techniques for transforming a wide variety of higher plant species are well known and described in the technical and scientific literature. See. e.g., Weising et al. Ann. Rev. Genet. 22:421-477 (1988). A DNA sequence coding for a protein can be combined with cis-acting (promoter) and trans-acting (enhancer) transcriptional regulatory sequences to direct the timing, tissue type and levels of transcription in the intended tissues of the transformed plant. Translational control elements can also be used. In some embodiments, a terminator sequence is included in the expression construct. An exemplary NOS terminator sequence is CCGATCGTTCAAACATTTGGCAATAAAGTTTCTTAAGATTGAATCCTGTTGCCGG TCTTGCGATGATTATCATATAATTTCTGTTGAATTACGTTAAGCATGTAATAATTA ACATGTAATGCATGACGTTATTTATGAGATGGGTTTTTATGATTAGAGTCCCGCA ATTATACATTTAATACGCGATAGAAAACAAAATATAGCGCGCAAACTAGGATAA ATTATCGCGCGCGGTGTCATCTATGTTACTAGATCGGGAATTGATCCCCCCTCGA CAG (SEQ ID NO: 80).

Also provided are host cell(s) comprising a heterologous egg cell-expressing promoter operably linked to a DWT1 polypeptide coding sequence, and optionally further comprising an expression cassette comprising a heterologous egg cell-expressing promoter operably linked to a Baby boom polypeptide coding sequence, as described herein. Exemplary host cells include, for example, prokaryotic (e.g., including but not limited to E. coli) cells or eukaryotic cells, and can for example plant, fungal, yeast, mammalian, insect, or other cells. Also provided as discussed above are plants comprising a heterologous egg cell-expressing promoter operably linked to a DWT1 polypeptide coding sequence, and optionally further comprising an expression cassette comprising a heterologous egg cell-expressing promoter operably linked to a Baby boom polypeptide coding sequence, as described herein.

Any method of introducing a first expression cassette comprising a heterologous egg cell-expressing promoter operably linked to a DWT1 polypeptide coding sequence, and a second expression cassette comprising a heterologous egg cell-expressing promoter operably linked to a Baby boom polypeptide coding sequence can be used. In some embodiments, both expression cassettes are introduced in one transformation. In other embodiments, a first expression cassette is introduced into a plant and then the resulting transformant is further transformed with the second expression caseate. See, e.g., the Example. In some embodiments, the expression cassettes as described herein are combined with suitable T-DNA flanking regions and introduced into a conventional Agrobacterium tumefaciens host vector. The virulence functions of the Agrobacterium tumefaciens host will direct the transfer of the T-DNA into plant cells when the cell is infected by the bacteria. Agrobacterium tumefaciens-mediated transformation techniques, including disarming and use of binary vectors, are well described in the scientific literature. See, for example, Horsch et al. Science 233:496-498 (1984), and Fraley et al. Proc. Natl. Acad. Sci. USA 80:4803 (1983). Alternatively other transformation methods can be used.

In some embodiments, transformation will be performed on embryonic plant tissue. For example, Agrobacterium tumefaciens can be co-cultivated with seed embryo-derived secondary calluses (see, e.g., Sallaud, C. et al., Theor. Appl. Genet. 106, 1396-1408 (2003); U.S. Pat. No. 10,584,345: EP0290395; and US2011/0212525). In some embodiments, transformation will be performed on somatic tissue. In some embodiments, transformation will be performed on plant protoplasts. Transformed cells can subsequently be selected (e.g., selecting for antibiotic resistance or other selectable marker introduced with the T-DNA or as otherwise known in the art). Primary transformed cells can subsequently be regenerated into plants.

The plant manipulated as described herein can be any plant species. In some embodiments, the plant is a dicot plant. In some embodiments the plant is a monocot plant. In some embodiments, the plant is a grass. In some embodiments, the plant is a cereal (e.g., including but not limited to Poaceae, e.g., rice, barley, wheat, maize). In some embodiments, the plant is a species of plant of the genus Abelmoschus, Allium, Apium, Amaranthus, Arachis, Arabidopsis, Asparagus, Atropa, Avena, Benincasa, Beta, Brassica, Cannabis, Capsella, Cica, Cichorium, Citrus, Citrullus, Capsicum, Carthamus, Cocos, Coffea, Cucumis, Cucurbita, Cynasa, Daucus, Diplotaxis, Dioscorea, Elais, Eruca, Foeniculum, Fragaria, Glycine, Gossypium, Helianthus, Heterocallis, Hordeum, Hyoscyamus, Ipomea, Lactuca, Lagenaria, Lepidium, Linum, Lolium, Luffa, Luzula, Lycopersicon, Malus, Manihot, Majorana, Medicago, Momodica, Musa, Nicotiana, Olea, Oryza, Panicum, Pastinaca, Pennisetum, Persea, Petroselinium, Phaseolus, Physalis, Pinus, Pisum, Populus, Pyrus, Prunus, Raphanus, Saccharum, Secale, Senecio, Sesamum, Sinapis, Solanum, Sorghum, Spinacia, Theobroma, Trichosantes, Trigonella, Triticum, Turritis, Valerianelle, Vitis, Vigna, or Zea.

As noted above, by introducing the two expression cassettes or otherwise inducing expression of Baby boom and DWT1 in egg cells, one can induce a high rate of parthenogenesis. For example, expression of both Baby boom and DWT1 in egg cells results in greater rates of parthenogenesis than expression of Baby boom alone, DWT1 alone, or in the absence of expression of either in egg cells. In some embodiments, the rate of parthenogenesis is at least, e.g., 40%, 50%, 60%, 70%, 80%, 85%, 90%, or 95%. Parthenogenesis results in a reduction (halving) of the number of chromosomes by delivering only one parent's chromosomes into the egg. In the absence of MiMe (discussed below), this means for example that a diploid parent plant will produce seed that is haploid, or for example a tetraploid plan would produce diploid seed. The percent of seed having only the chromosomes of one parent represents the rate of rate of parthenogenesis.

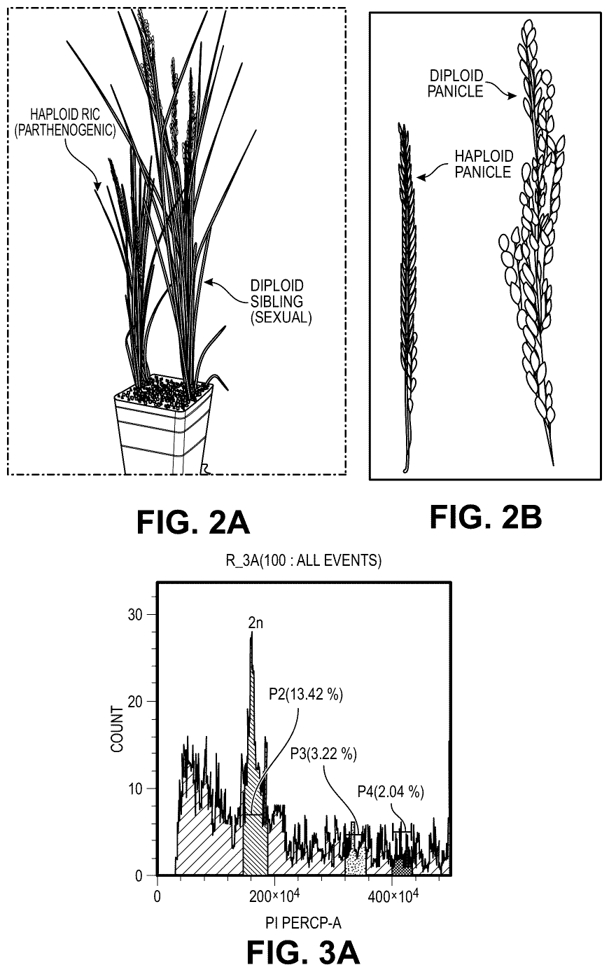

In some embodiments, a portion of the seed or seed coat is removed and a genetic test is performed to determine whether the seed is haploid prior to germination. In other embodiments, the seeds are germinated and the resulting progeny plants are screened for those that are haploid, either by testing their genotype or by observation (haploid plants in many cases are smaller than diploid progeny, see FIG. 2). Optionally, one can physically separate progeny into groups of only haploid plants, optionally discarding diploid progeny or otherwise physically separating diploid progeny from haploid progeny.

Once generated, haploid plants can be used for a variety of useful endeavors, including but not limited to the generation of doubled haploid plants, which comprise an exact duplicate copy of chromosomes. Such doubled haploid plants are of particular use to speed plant breeding, for example. A wide variety of methods are known for generating doubled haploid organisms from haploid organisms.

Somatic haploid cells, haploid embryos, haploid seeds, or haploid plants produced from haploid seeds can be treated with a chromosome doubling agent. Homozygous double haploid plants can be regenerated from haploid cells by contacting the haploid cells, including but not limited to haploid callus, with chromosome doubling agents, such as colchicine, anti-microtubule herbicides, or nitrous oxide to create homozygous doubled haploid cells.

Methods of chromosome doubling are disclosed in, for example, U.S. Pat. No. 5,770,788; 7,135,615, and US Patent Publication No. 2004/0210959 and 2005/0289673; Antoine-Michard. S. et al., Plant Cell. Tissue Organ Cult., Dordrecht, the Netherlands, Kluwer Academic Publishers 48(3): 203-207 (1997); Kato, A., Maize Genetics Cooperation Newsletter 1997, 36-37; and Wan, Y. et al., Trends Genetics 77:889-892 (1989). Wan, Y. et al., Trends Genetics 81:205-211 (1991), the disclosures of which are incorporated herein by reference. Methods can involve, for example, contacting the haploid cell with nitrous oxide, anti-microtubule herbicides, or colchicine. Optionally, the haploids can be transformed with a heterologous gene of interest, if desired.

Double haploid plants can be further crossed to other plants to generate F1, F2, or subsequent generations of plants with desired traits.

In some embodiments, one can make clonal plants from a parent plant expressing BABYBOOM and DWT1 in egg cells as described herein. This can be achieved, for example, when the parent plant, which is parthenogenic as described above, produces gametes (e.g., egg or pollen cells) having the same number of chromosomes as somatic cells in the plant. Thus for example, if the plant is diploid (the somatic tissue is diploid) then the gametes are also diploid. This can be achieved in various ways, for example by inducing a “mitosis instead of meiosis” (MiME) phenotype in the parent plant (in addition to the expression of BABYBOOM). See. e.g., US Patent Publication No. 2012/0042408 and PCT Publication No. WO 2012/075195. Seed from a plant expressing BABYBOOM and DWT1 in egg cells, and having mutations that induce the MiMe phenotype, will be clonal to the parent plant. Mutations that induce MiMe phenotype are known and can be introduced into the plant as desired. In some embodiments, a RNA-guided nuclease and sufficient guide RNAs are expressed in the plant to induce mutations that cause the MiMe phenotype.

As noted above, in some embodiments the plant also comprises an expression cassette comprising a promoter operably linked to an RNA-guided nuclease. The RNA-guided nuclease can recognize a sequence of a target nucleic acid (e.g., via an RNA guide), bind to the target nucleic acid, and modify the target nucleic acid. The RNA-guided nuclease has nuclease activity. For example, the RNA-guided nuclease can modify the target nucleic acid by cleaving the target nucleic acid. After the action of the nuclease at the beginning of a coding sequence (as targeted by a gRNA), the introduction of inserts or deletions by the error-prone non-homologous end joining repair of double-strand breaks (DSBs) introduces frame-shift mutations and for example subsequent premature stop codons, leading to mRNA elimination by nonsense-mediated mRNA decay. For example, the Cas nuclease can direct cleavage of one or both strands at a location in a target nucleic acid. Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, variants thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015: 40(1): 58-66). Type II Cas nucleases include Cas1, Cas2, Csn2, Cas9, and Cfp1. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI No. WP_011681470.

Cas nucleases, e.g., Cas9 nucleases, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai. Streptococcus mutans. Listeria innocua. Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp, Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp, Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp, Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp, Multocida, Sutterella wadsworthensis, proteobacterium, Legionella pneumophila, Parasutterella excrementihominis, Wolinella succinogenes, and Francisella novicida.

Cas9 protein refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas9 can be a fusion protein, e.g., the two catalytic domains are derived from different bacteria species.

In some embodiments, a Cas protein can be a Cas protein variant. For example, useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single strand break or nick. In some embodiments, the Cas9 nuclease can be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). Non-limiting examples of Cas9 nucleases or nickases are described in, for example, U.S. Pat. No. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9 (1.0)), and SpCas9 (K848A/K1003A/R1060A) (also referred to as eSpCas9 (1.1)) variants described in Slaymaker et al., Science, 351 (6268): 84-8 (2016), and the SpCas9 variants described in Kleinstiver et al., Nature, 529 (7587): 490-5 (2016) containing one, two, three, or four of the following mutations: N497A, R661A, Q695A, and Q926A (e.g., SpCas9-HF1 contains all four mutations).

The promoter operably linked to the sequence encoding the RNA guided nuclease can be a constitutive promoter or an egg-specific promoter or be otherwise selected such that the RNA guided nuclease is expressed in egg cells. Examples of constitutive promoters include the cauliflower mosaic virus (CaMV) 35S transcription initiation region, the 1′- or 2′-promoter derived from T-DNA of Agrobacterium tumefaciens, the parsley UBI promoter (Kawalleck et al., Plant Mol Biol. (1993 February) 21 (4): 673-84), RPS5 (Hiroki Tsutsui et al. Plant and Cell Physiology (2016)): 2X35SΩ (Belhaj, Khaoula, et al. Plant methods 9.1 (2013): 39): AtUBI10 (Callis J, et al. Genetics 139:921-939 (1995)); SIUBI10 (Dahan-Meir. Tal, et al. The Plant Journal (2018)); G10-90 (Ishige, Fumiharu, et al. The Plant Journal 18.4 (1999): 443-448) and other transcription initiation regions from various plant genes known to those of skill. In some embodiments, each expression cassette in the single construct uses a different promoter.

The RNA-guided nuclease will be expressed with a sufficient set of expression cassettes directing expression of guide RNAs (gRNAs) to induce a meiosis-to-mitosis phenotype. Plant genes to be targeted to obtain a MiMe phenotype are known and are also described below. In general, expression of a single guide RNA per gene can be sufficient to reduce expression of each target gene, but if desired, two or more guide RNA can be targeted to one of more of the genes to further reduce its expression.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the DNA targeting sequence is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence. The guide sequence can be used in a single-guide RNA (sgRNA) as described below, or in a split crRNA+tracrRNA construct.

In some embodiments, the targeted nuclease (e.g., a Cas protein) is guided to its target DNA by a single-guide RNA (sgRNA). An sgRNA is a version of the naturally occurring two-piece guide RNA (crRNA and tracrRNA) engineered into a single, continuous sequence. An sgRNA typically contains (1) a guide sequence (e.g., the crRNA equivalent portion of the sgRNA) that targets the Cas protein to the target DNA, and (2) a scaffold sequence that interacts with a nuclease such as a Cas protein (e.g., the tracrRNAs equivalent portion of the sgRNA). An sgRNA may be selected using a software. As a non-limiting example, considerations for selecting an sgRNA can include, e.g., the PAM sequence for the Cas9 protein to be used, and strategies for minimizing off-target modifications. Tools such as NUPACK® and the CRISPR Design Tool can provide sequences for preparing the sgRNA, for assessing target modification efficiency, and/or assessing cleavage at off-target sites.

The guide sequence in the sgRNA may be complementary to a specific sequence within a target DNA. The 3′ end of the target DNA sequence can be followed by a PAM sequence. Approximately 20 nucleotides upstream of the PAM sequence is the target DNA. In general, a Cas9 protein or a variant thereof cleaves about three nucleotides upstream of the PAM sequence. The guide sequence in the sgRNA can be complementary to either strand of the target DNA.

The promoter operably linked to the sequence encoding the guide RNA can be a constitutive promoter or an egg-specific promoter or be otherwise selected such that the guide RNA is expressed in egg cells. Examples of constitutive promoters include the cauliflower mosaic virus (CaMV) 35S transcription initiation region, the 1′- or 2′-promoter derived from T-DNA of Agrobacterium tumefaciens, the parsley UBI promoter (Kawalleck et al., Plant Mol Biol. (1993 February) 21(4): 673-84), RPS5 (Hiroki Tsutsui et al. Plant and Cell Physiology (2016)): 2X35SΩ (Belhaj, Khaoula, et al. Plant methods 9.1 (2013): 39); AtUBI10 (Callis J, et al. Genetics 139:921-939 (1995)); SIUBI10 (Dahan-Meir, Tal, et al. The Plant Journal (2018)); G10-90 (Ishige, Fumiharu, et al. The Plant Journal 18.4 (1999): 443-448) and other transcription initiation regions from various plant genes known to those of skill.

Genes necessary to knock out for generation of plants having the MiME phenotype are known. See. e.g., US Patent Publication No. 2012/0042408; US Patent Publication No. 2014/0298507, and PCT Publication No. WO 2012/075195. A plant having the MiMe (mitosis instead of meiosis) genotype is a plant in which a deregulation of meiosis results in a mitotic-like division and in which meiosis is replaced by mitosis. Plants having the MiMe genotype produce functional (e.g., diploid) gametes that are genetically identical to their parent. Exemplary MiMe plants combine phenotypes of (1) no second meiotic division, (2) no recombination and (3) modified chromatid segregation. MiMe plants are exemplified by MiMe-1 plants as described by d'Erfurth, I. et al. PLOS Biol 7, e1000124 (2009) and WO2001/079432) and MiMe-2 plants as described by d'Erfurth, I. et al. PLOS Genet 6, e1000989 (2010). In some embodiments, the MiMe phenotype is induced by inhibiting or mutating OSD1 or an ortholog thereof, REC8 or an ortholog thereof, and at least one of SPO11 or PRD1, or PRD2 or PRD3/PAIR1 (see, e.g., Mieulet D., Cell Res. 2016 November; 26(11): 1242-1254).

Exemplary MiMe-1 plants combine inactivation of the OSD1 gene, with the inactivation of two or more other genes, one which encodes a protein necessary for efficient meiotic recombination in plants (e.g., SPO11-1, SPO11-2, PRD1, PRD2, or PAIR1), and whose inhibition eliminates recombination and pairing (see, e.g., Grelon M, et al. EMBO Journal 20, 589-600 (2001)), and another which encodes a protein necessary for the monopolar orientation of the kinetochores during meiosis, e.g., REC8, and whose inhibition modifies chromatid segregation (see, e.g., Chelysheva L, et al., Journal of Cell Science 118, 4621-4632. (2005)]. Exemplary MiMe-2 plants combine inactivation of the TAM gene with the inactivation of two or more other genes, one which encodes a protein necessary for efficient meiotic recombination in plants (e.g., SPO11-1, SPO11-2, PRD1, PRD2, or PAIR1), and whose inhibition eliminates recombination and pairing, and another which encodes a protein necessary for the monopolar orientation of the kinetochores during meiosis, e.g., REC8, and whose inhibition modifies chromatid segregation.

Exemplary OSD1 gene sequences include, e.g., those described in US Patent Publication No. 2014/0298507 and rice and Arabidopsis OSD1 protein sequences as provided in SEQ ID NOS: 34 and 36, respectively.

Exemplary TAM gene sequences are described in, e.g., US Patent Publication No. 2014/0298507. Arabidopsis TAMI protein sequence is provided as SEQ ID NO:46. Illustrative rice Cyclin-A1 protein sequences are provided as SEQ ID NOS: 48, 50, 52, 54, and 56. Illustrative Cyclin-A3 protein sequences are provided as SEQ ID NOS: 58 and 60.

Exemplary Arabidopsis DYAD cDNA coding sequence and the sequence of the protein encoded by the nucleic acid are provided as SEQ ID NOS: 70 and 71, respectively. Exemplary rice DYAD homolog (SWITCH1) protein sequences are provide as SEQ ID NOS: 64, 66, and 68.

Examples of SPO11-1 and SPO11-2 proteins are provided in US Patent Publication No. 2014/0298507. An illustrative Arabidopsis SPO11-2 protein sequence is provided as SEQ ID NO:40.

Arabidopsis PAIR1 is described in, e.g., US Patent Publication No. 2014/0298507. An exemplary rice PAIR1 protein sequence is provided as SEQ ID NO:38.

Exemplary rice and Arabidopsis REC8 protein sequences are provided as SEQ ID NOS: 60 and 62, respectively.

In some embodiments, sufficient expression cassettes to produce the MiMe phenotype include at least one expression cassette comprising a promoter operably linked to one or more guide RNA targeting a gene or coding sequence encoding (a) a TAM (Cylin A CYCA1; 2) or DYAD protein or ortholog thereof: (b) a protein involved in initiation of meiotic recombination in plants exemplified herein as SPO11-1; SPO11-2; PRD; PRD2; or PAIR1 (also called PRD3) or ortholog thereof; and (c) a protein necessary for the monopolar orientation of the kinetochores during meiosis for example REC8 protein or ortholog thereof. Orthologs have the functionality of the proteins described herein but are from different plant species. Orthologs can be substantially identical to the polypeptides as provide herein or can otherwise be selected from genomic databases.

In some embodiments, sufficient expression cassettes to produce the MiMe phenotype include at least one expression cassette comprising a promoter operably linked to one or more guide RNA targeting a gene or coding sequence encoding (a) an OSD 1 protein or ortholog thereof: (b) a protein involved in initiation of meiotic recombination in plants exemplified herein as SPO11-1: SPO11-2: PRD: PRD2; or PAIR1 (also called PRD3) or ortholog thereof; and (c) a protein necessary for the monopolar orientation of the kinetochores during meiosis, for example REC8 protein or ortholog thereof.

### EXAMPLE

The ability of another male-expressed transcription factor DWARF TILLER1 (OsDWT1) to induce parthenogenesis if expressed in the egg cell was tested. The DWT1 transcription factor is encoded by a member of the WUSCHEL-Homebox or WOX gene family (Wang et al. 2014). The parthenogenetic ability of OsDWT1 either alone, or in combination with OsBBM1, was test for the ability to increase the parthenogenesis efficiency in rice.

### Methods

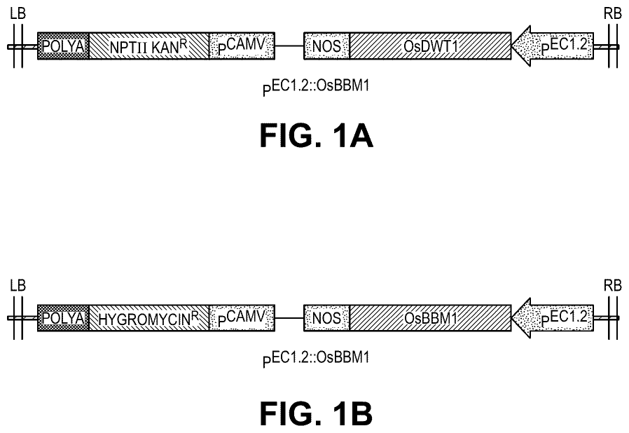

OsDWT1 CDS was cloned under Arabidopsis and rice egg-cell specific promoters (see sequences in List 2 below) for expression the egg cells (FIG. 1, attached). OsDWT1 CDS was initially amplified from cDNAs from rice callus tissues with primers listed in List 1. The CDS was amplified in three fragments which were joined at two unique (NcoI and NheI) restriction sites to complete the sequence. The complete CDS was then cloned into pCAMBIA2300 based binary vectors in which egg cell promoters from Arabidopsis and rice, and Nos transcription terminators were already cloned (FIG. 1A). The binary constructs containing the egg-cell specific OsDWT1 expression cassettes were super transformed, using Agrobacterium mediated transformation, into seeds homozygous for pEC1.2::OsBBM1 from line #8c (FIG. 1B; Khanday et al., 2019). Thirteen T0 transgenic plants were raised with the pEC1.2::OsDWT1:NosT construct. The primary T0 transgenic plants were confirmed for the presence of the transgene by PCR amplifying the NptII selection maker. Ten T0 lines, hemizygous for the pEC1.2::OsDWT1:NosT transgene were analyzed for their capacity to induce haploidy. T1 seeds were germinated on ½ MS media containing 300 mg/L of G418 in a growth chamber with 16/8 hour light/dark cycle at 25° C. for 12 days. In parallel, T1 seeds were also germinated on ½ MS media without G418. The germinated seedlings, resistant to G418 (both hemizygous and homozygous for pEC1.2::OsDWT1:NosT) and those germinated on media without G418 were transferred to the greenhouse after 12 days. The seedlings were allowed to undergo flowering transition and phenotypes were scored after the panicles fully emerged.

### Results

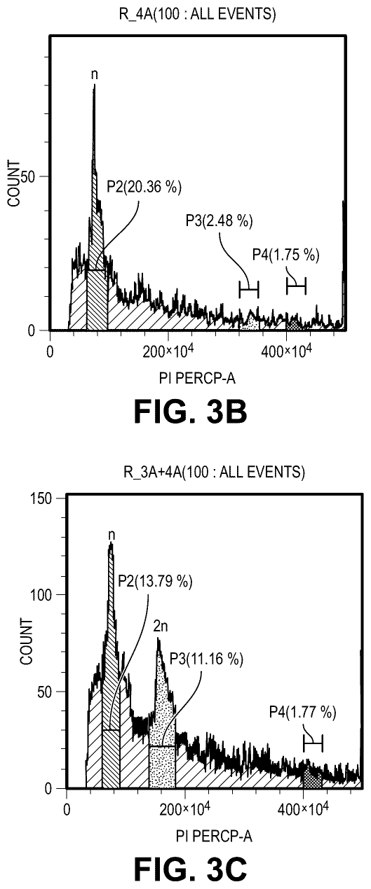

When OsDWT1 alone was expressed in the egg cell, it was unable to induce parthenogenesis. We then tested the possibility of OsBBM1 and OsDWT1 acting synergistically to increase the parthenogenesis efficiency in rice. Several independent TO lines were generated by Agrobacterium transformation (see Methods) and their T1 progenies were analyzed (See methods for details above). Depending upon the efficiency of parthenogenesis, the T1 progenies are expected to be a mixture of haploids (parthenogenetic progeny) and diploids (sexual progeny). The ploidy determination was carried out by observing the plant phenotype. The haploids are dwarf with narrow leaves compared to diploids, and sterile due to meiotic defects (FIG. 2). The final confirmation of ploidy was done by flow cytometry (FIG. 3). As expected, haploid progenies displayed a flow cytometric peak at 80 (FIG. 3B), whereas diploid peaks were double the size of haploids, at about 160 (FIG. 3A). Thus, these results confirm the genome size of haploid and diploid progenies. The haploid induction frequencies were calculated by the number of haploid progenies obtained, divided by the total number of seedlings germinated. From the hemizygous TO mother plant (line #12b), the induction frequency of haploids in T1 generation was calculated to be 45.8% (Table 1A). Since only half the egg cells of the hemizygous TO parent would have inherited the pEC1.2::OsDWT1:NosT transgene, the actual parthenogenesis efficiency (% of egg cells carrying the transgene that underwent parthenogenesis and produced haploids) will be higher, i.e. about 92%.

We then identified homozygous pEC1.2::OsDWT1:NosT diploid T1 individuals by germination on G418: All T2 progeny of homozygotes will be resistant to this antibiotic, whereas hemizygous individuals will produce ¾ resistant and ¼ sensitive T2 progeny. The T2 progeny of these homozygous plants were analyzed to estimate parthenogenesis efficiency. The haploid frequency in these T2 progenies from homozygous T1 mother plants increased to 91% (Table 1A). As the T1 parents were homozygous for the transgene, the parthenogenesis efficiency is equal to the haploid frequency, i.e., 91%. This is a greater than 3-fold increase over the 15-29% efficiency of the parent pEC1.2::OsBBM1 #8c line that was used for transformation of the pEC1.2::OsDWT1:NosT construct (Table 1A).

We also screened for diploid T1 progenies in which pEC1.2::OsDWT1:NosT transgene had segregated out (negative for the pEC1.2::OsDWT1:NosT transgene) from plants germinated on ½ MS media without G418. The parthenogenesis frequency in T2 progenies from these pEC1.2::OsDWT1:NosT negative plants was found to be 20.1% (Table 1A). Thus, the combination of OsBBM1 and OsDWT1 act synergistically to increase the parthenogenesis efficiency, in this instance by 4.5-fold over OsBBM1 alone, and thereby the number of haploid progenies.

We carried out similar analyses for an independent transgenic line #7d (Table 1B). Similar to line #12b, the parthenogenesis efficiencies were much higher than with just the pEC1.2::OsBBM1 transgene. The haploid frequency of pEC1.2::OsDWT1:NosT hemizygous TO mother plants increased to 48.2%, and that of homozygous T1 mother plants increased to 86.4% (Table 1B). The haploid frequency from sibling T1 mother plants of the same line that were negative for presence of the pEC1.2::OsDWT1:NosT transgene, i.e. carrying only pEC1.2::OsBBM1, was 5.5% (Table 1B). Thus, in this instance, the combination of OsBBM1 and OsDWT1 acting synergistically increased the parthenogenesis efficiency by 15-fold over OsBBM1 alone. Together, these results show that OsDWT1 when expressed in the egg cell, increases the parthenogenetic capacity of OsBBM1, by up to 4 to 15-fold over OsBBM1 alone.

### Discussion

Heterosis refers to the enhanced vigor in F1 progenies compared to their inbred parents. F1 hybrids have been used to substantially increase crop yields. However, due to genetic segregation resulting from sexual reproduction, F1 hybrid seeds need to be created afresh for cultivation during every sowing season, as high-yielding hybrids cannot be maintained through normal seed propagation. To circumvent this problem and to fix the vigor in hybrid crops, we combined the parthenogenesis ability of OsBBM1 with a method of substituting mitosis for meiosis, thus bypassing segregation and fertilization to develop a method of synthetic apomixis or clonal seed formation (Khanday et al., 2019).

However, low parthenogenesis frequency (˜29%) remains a bottleneck for this technology for field application, which would require frequencies of at least 80% parthenogenesis to be commercially useful. Through this invention, we have attained parthenogenesis efficiencies of 86 to 91%, which will pave the way for synthetic apomixis technology to be introduced into the farmer's field for hybrid crop cultivation.

TABLE 1
Increase in parthenogenesis frequencies
by DWT1 expressed in rice egg cell
Frequency of parthenogenesis in T1 generation
measured by % of haploids in T2 Progeny

| | | |
|---|---|---|
| | Progeny | |
| | (Genera- | % |
| Genotype of plants | tion) | Haploids |

A. Transformant #12b
T0 genotype
eB1/eB1; eDT/— (Homozygous for BBM1
transgene, hemizygous for DWT1 transgene)
% Haploids in T1 progeny of T0 parent: 45.8% (n = 83)

| eDT/eDT; eB1/eB1 | T2 | 91.0% | (n = 134) |
|---|---|---|---|
| (Homozygous for DWT1 and BBM1) | | | |
| eDT/—; eB1/eB1 | T2 | 42.3% | (n = 156) |
| (Hemizygous for DWT1 transgene, | | | |
| Homozygous for BBM1 transgene) | | | |
| —/—; eB1/eB1 | T2 | 20.1% | (n = 144) |
| (No DWT1 transgene, Homozygous for | | | |
| BBM1 transgene) | | | |
| Baseline comparison: original eB1/eB1 | — | 15%-29% | |
| line used for transformation | | | |
| (Homozygous for BBM1 transgene) | | | |

B. Transformant #7d
T0 genotype
eB1/eB1; eDT/— (Homozygous for BBM1
transgene, hemizygous for DWT1 transgene)
% Haploids in T1 progeny of T0 parent: 48.2% (n = 56)

| eDT/eDT; eB1/eB1 | T2 | 86.4% | (n = 88) |
|---|---|---|---|
| (Homozygous for DWT1 and BBM1) | | | |
| eDT/—; eB1/eB1 | T2 | 36.0% | (n = 114) |
| (Hemizygous for DWT1 transgene, | | | |
| Homozygous for BBM1 transgene) | | | |
| —/—; eB1/eB1 | T2 | 5.5% | (n = 165) |
| (No DWT1 transgene, Homozygous for | | | |
| BBM1 transgene) | | | |
| Baseline comparison: original eB1/eB1 | — | 15%-29% | |
| line | | | |
| (Homozygous for BBM1 transgene) | | | |

Symbols:
eDT = Transgene pEC1.2::DWT1
eB1 = Transgene pEC1.2::BBM1

List 1: DNA Primers for PCR amplification of
sequences
Primers for rice egg cell promoter pECA1

| | |
|---|---|
| REG1 F | ATG GAA TGA TGG ATG AAT GTT CAC |
| | (SEQ ID NO: 81) |
| REG1 R | GG TTT TTC TTT CTA GCT TTG CTG |
| | (SEQ ID NO: 82) |

Primers for amplification of Arabidopsis EC1.1
promoter

| | |
|---|---|
| EC1.1 F | GTTGCCTTATGATTTCTTCGGTTT |
| | (SEQ ID NO: 83) |
| EC1.1 R | TTCTCAACAGATTGATAAGGTCGAAA |
| | (SEQ ID NO: 84) |

Primers for amplification of Arabidopsis EC1.2
promoter

| | |
|---|---|
| DD45PstI F | CTGCAGAAATGTTCCTCGCTGACGTA |
| | (SEQ ID NO: 85) |
| DD45SalI R | GTCGACTATTCTTTCTTTTTGGGGTTTTTG |
| | (SEQ ID NO: 86) |

Primers for amplification of DWT1

| | |
|---|---|
| DWT1 FF | ATG GCG TCG TCG AAC AGG CAC |
| | (SEQ ID NO: 87) |
| DWT1 R2 | GTCCATGGCCGTCGTCACG |
| | (SEQ ID NO: 88) |
| DWT1 F2 | CGT GAC GAC GGC CAT GGA C |
| | (SEQ ID NO: 89) |
| DWT1 R3 | CAGTCAGGCTAGCGGTGGC |
| | (SEQ ID NO: 90) |
| DWT1 F3 | GCC ACC GCT AGC CTG ACT G |
| | (SEQ ID NO: 91) |
| DWT1 RL | TTACATGACAACAATGTAGACGGC |
| | (SEQ ID NO: 92) |

### REFERENCES

1. Anderson S N, Johnson C S, Chesnut J, Jones D S, Khanday I, Woodhouse M, Li C, Conrad L J, Russell S D, Sundaresan V (2017) The zygotic transition is initiated in unicellular plant zygotes with asymmetric activation of parental genomes. Developmental Cell 2017, 43:349-358.e4.
2. Khanday, I., Skinner, D., Yang, B., Mercier, R., Sundaresan, V. (2019). A male-expressed rice embryogenic trigger redirected for asexual propagation through seeds. Nature 565:91-95 DOI: doi.org/10.1038/s41586-018-0785-8
3. Wang W, Li G, Zhao J, Chu H, Lin W, et al. (2014) DWARF TILLER1, a WUSCHEL-Related Homeobox Transcription Factor, Is Required for Tiller Growth in Rice. PLOS Genet 10(3): e1004154. doi:10.1371/journal.pgen. 1004154

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

SEQUENCES

SEQ ID NO: 1
DWT1 Oryza sativa Japonica

| | |
|---|---|
| 1 | massnrhwps mfrskhatqp wqtqpdmags ppsllsgssa gsaggggysl ksspfssvge |
| 61 | ervpdpkprw nprpeqiril eaifnsgmvn pprdeiprir mqlqeygqvg danvfywfqn |
| 121 | rksrsknklr sggtgraglg lggnrasapa aahreavaps ftppppilpa pqpvqpqqql |
| 181 | vspvaaptss sssssdrssg sskparatst qamsvttamd llsplaaach qqmlyqgqpl |
| 241 | esppapapkv hgivphdepv flqwpqspcl savdlgaail ggqymhlpvp apqppsspga |
| 301 | agmfwglcnd vqapnntghk scawsaglgq hwcgsadqlg lgkssaasia tvsrpeeahd |
| 361 | vdatkhgllq ygfgittpqv hvdvtssaag vlppvpssps ppnaavtvas vaatasltdf |
| 421 | aasaisagav annqfqglad fglvagacsg agaaaaaaap eagssvaavv cvsvagaapp |
| 481 | lfypaahfnv rhygdeaell ryrggsrtep vpvdesgvtv eplqqgavyi vvm |

SEQ ID NO: 2 DWL2
Oryza sativa Japonica

| | |
|---|---|
| 1 | maspnrhwps mfrsnlacni qqqqqpdmng ngsssssfll spptaattgn gkpsllssgc |
| 61 | eegtrnpepk prwnprpeqi rilegifnsg mvnpprdeir rirlqlqeyg qvgdanvfyw |
| 121 | fqnrksrtkn klraaghhhh hgraaalpra sappstnivl psaaaaaplt pprrhllaat |
| 181 | sssssssdrs sgssksvkpa aaalltsaai dlfspapapt tqlpacqlyy hshptplard |
| 241 | dqlitspess slllqwpasq ympatelggv lgssshtqtp aaitthpsti spsvllglcn |
| 301 | ealgqhqqet mddmmitcsn pskvfdhhsm ddmsctdavs avnrddekar lgllhygigv |
| 361 | taaanpaphh hhhhhhlasp vhdavsaada staamilpft ttaaatpsnv vatssaladq |
| 421 | lqglldagll qggaapppps atvvavsrdd etmctkttsy sfpatmhlnv kmfgeaavlv |
| 481 | rysgepvlvd dsgvtveplq qgatyyvlvs eeavh |

SEQ ID NO: 3. DWL1
Oryza sativa Japonica

| | |
|---|---|
| 1 | mtllavivfg gggggsrssa lpslstgvag vegsatgpwl pesltlnaat hripstspls |
| 61 | ipqtltitrd ppypmlprsh ghrtggggfs lksspfssvg eervpdpkpr rnprpeqiri |
| 121 | leaifnsgmv npprdeipri rmqlqeygqv gdanvfywfq nrksrsknkl rsggtgragl |
| 181 | glggnrasep paaatahrea vapsftpppi lppqpvqpqq qlvspvaapt slsssssdrs |
| 241 | sgsskparat ltqamsvtaa mdllsplrrs arprqeqrhv |

SEQ ID NO: 4 Maize DWT1

| | |
|---|---|
| 1 | massfnnkts hwpsmfrskh aaepwqaqpd isssppslls gggsssttti grclkhplsg |
| 61 | ysggeertpd pkprwnprpe qirileaifn sgmvnpprde iprirmrlqe ygqvgdanvf |
| 121 | ywfqnrksrs knkqrtgqlg lglarapgcg aaappvtpqp liqnqfqvla spaqapasss |
| 181 | ssssdrssgs skpapqpmsa taaamnfpgp lgaacaqmyy qahpvapvsa lpahkvqdpv |
| 241 | asdepvfqpw lqgyflsaae vasilggqyr hdvpvqqqpp atlpagaflg lynevteptv |
| 301 | tghrtcawgp aglgqfwpvg gadhhqhhkh nttaatntva rdaahehatt lgllqygfea |
| 361 | saametasaa vplaaspgta asvataglts lpastnavvv nydllqglav pgsgsgavgv |
| 421 | stggappvav aaaptaaqeg vvvalcitds vtgksvahnv aaarldvraq fgeaavllra |
| 481 | vgdrggldlv pvpvdalgct veplqhgafy yvlv |

SEQ ID NO: 5 Maize DWL2

| | |
|---|---|
| 1 | massnrhwps myrsslacnf qqpqpqpdmn nggksslmss rceenggrnp eprprwnprp |
| 61 | eqirilegif nsgmvnpprd eirrirlqlq eygpvgdanv fywfqnrksr tkhklraagq |
| 121 | lqpsgsgrsa lqaracapap vtpprnlqla aaapvappts ssssssdrss gssssksvtv |
| 181 | tpttavalas pagaapaavf rqqgvmptta mdlltplpss saalaarqly yqyhsqimap |
| 241 | aappmpdtvi aspeqflpqw qqggqqhyyl patelggvld ghshhthepp aaihrpvsls |
| 301 | psvlfglcne alrqdycadi svvptkglgh ghqfwnsttc gsdmgnsnsk idavsavird |
| 361 | deksrlgllh yyglagattt aaaavapapl aadaaagtat llpssaasdq lqglldaagl |
| 421 | lmgetpptpt atvvavarda vtcaatataq fsvpasmrld vrlafgeaal larhtgeavp |
| 481 | vdesgvtvep lqqdtlyyvl mqatnn |

SEQ ID NO: 6 Maize DWL1

| | |
|---|---|
| 1 | masssfnnsh wpsmfrskha aepcqttqpd isssppslls aggasttttt grclkhsisv |
| 61 | ggeerapdpk prwnprpeqi rileaifnsg mvnpprdeip rirmrlqqyg qvgdanvfyw |
| 121 | fqnrksrskn klrsstagtg rlglqglara pgrgaaaapp pveppplvqn qfhmlaspaq |
| 181 | aptsssssss drssgsskpa aepampataa pmdllgplaa acpqmyyqgs pvapahkvld |
| 241 | lvasvepvfq pwpqgyclsa aevatilggq ymhvpvqqqp paplpagall glcndvtept |
| 301 | avvtghktca wgpaglgqsw pcggadhhqp gknnntaare laheddatkl gllqygfgat |
| 361 | tameaapava plaaspagga vtmasvsast agltgfpast ngvvanydll qglavpggga |
| 421 | gagrapaava vaadaaptaa qegvvalcit dsitgksvah nvaaarldvr aqfgeaavll |
| 481 | rcggergldl epvpvdasgc tveplqrgaf yyvll |

SEQ ID NO: 7: DWT1 = DWARF TILLER1 Nucleotide sequence
ATGGCGTCGTCGAACAGGCACTGGCCGAGCATGTTCAGGTCGAAGCACGCCACGCAGCCG
TGGCAGACGCAGCCTGACATGGCCGGGTCGCCGCCCTCCCTCCTCTCCGGCTCCTCCGCC
GGCAGCGCCGGCGGCGGCGGCTACTCCCTCAAGTCGTCGCCCTTCTCGTCAGTGGGCGAG
GAGAGGGTTCCGGACCCGAAGCCGCGGTGGAACCCGCGGCCGGAGCAGATCCGGATCCTG
GAGGCGATCTTCAACTCCGGCATGGTCAACCCGCCGCGCGACGAGATCCCGCGCATCCGC
ATGCAGCTGCAGGAGTACGGCCAGGTCGGCGACGCCAACGTCTTCTACTGGTTCCAGAAC
CGCAAGTCCCGCTCCAAGAACAAGCTGCGCTCCGGCGGGACAGGCCGCGCGGGGCTCGGC
CTCGGCGGCAACCGGGCCTCCGCGCCGGCGGCGGCGCACCGGGAGGCCGTGGCGCCGTCG
TTCACGCCGCCGCCACCAATCCTCCCGGCGCCCCAGCCGGTGCAGCCGCAGCAGCAGCTT
GTCTCGCCTGTGGCGGCGCCTACCTCGTCGTCGTCTTCCTCCTCCGACCGTTCGTCCGGG
TCCAGCAAGCCTGCGAGGGCTACGTCGACGCAGGCGATGTCCGTGACGACGGCCATGGAC
CTGCTCTCGCCGCTCGCCGCGGCGTGCCACCAGCAGATGCTCTATCAAGGCCAGCCACTG
GAGTCGCCGCCGGCGCCTGCTCCCAAAGTGCACGGCATCGTGCCACACGACGAGCCGGTC
TTCCTGCAGTGGCCGCAGAGCCCCTGCCTGTCGGCCGTCGACCTCGGCGCCGCCATTCTT
GGCGGCCAGTACATGCACCTGCCGGTGCCCGCTCCGCAGCCACCGTCGTCGCCGGGCGCG
GCGGGCATGTTCTGGGGGCTCTGCAACGACGTGCAAGCGCCAAACAACACCGGCCACAAG
AGCTGCGCCTGGAGCGCCGGGCTCGGCCAGCACTGGTGCGGCTCCGCCGATCAGCTCGGC
CTCGGCAAGAGCAGCGCGGCGTCGATCGCCACCGTGTCTAGGCCGGAGGAGGCGCACGAC
GTCGACGCCACGAAGCACGGTCTGCTACAGTACGGCTTTGGCATCACCACGCCGCAAGTG
CACGTGGACGTTACCTCCTCGGCTGCTGGCGTTCTGCCTCCTGTTCCGTCCTCGCCGTCG
CCGCCGAACGCCGCCGTCACCGTCGCGAGCGTGGCCGCCACCGCTAGCCTGACTGATTTT
GCTGCAAGTGCTATATCTGCTGGCGCCGTCGCTAACAATCAGTTTCAAGGTCTCGCGGAT
TTCGGGCTCGTCGCCGGCGCCTGCTCCGGCGCCGGAGCCGCCGCCGCCGCCGCCGCGCCC
GAGGCGGGCAGTTCCGTGGCCGCGGTTGTGTGCGTCAGCGTCGCGGGCGCCGCGCCGCCG
CTCTTCTACCCGGCCGCGCACTTCAACGTGAGGCACTACGGCGACGAGGCCGAGCTGCTC
CGCTACAGAGGAGGCAGCCGCACGGAGCCTGTGCCCGTCGACGAGTCGGGCGTCACCGTC
GAGCCGCTCCAGCAGGGCGCCGTCTACATTGTTGTCATGTAA
SEQ ID NO: 8 DWL2 = DWARF TILLER-LIKE2 Nucleotide sequence
ATGGCCTCACCGAACAGGCACTGGCCGAGCATGTTCAGGTCCAATCTTGCCTGCAACATC
CAGCAGCAGCAGCAGCCTGACATGAACGGCAACGGCAGCTCGTCCTCTTCCTTCCTCCTC
TCGCCACCTACTGCTGCGACCACCGGCAACGGCAAGCCCTCCTTGCTCTCCTCAGGGTGT
GAGGAGGGGACGAGGAATCCGGAGCCGAAGCCGCGGTGGAACCCGAGGCCGGAGCAGATA
AGGATACTGGAGGGGATCTTCAACTCCGGGATGGTGAACCCGCCGCGCGACGAGATCCGC
CGCATCCGCCTGCAGCTGCAGGAGTACGGCCAGGTCGGCGACGCCAACGTCTTCTACTGG
TTCCAGAACCGCAAGTCCCGCACCAAGAACAAGCTGCGCGCCGCCGGCCACCACCACCAC
CACGGCCGCGCCGCCGCCCTGCCGCGCGCGTCGGCGCCGCCGTCGACGAACATCGTACTC
CCCTCTGCAGCGGCGGCGGCGCCCTTGACGCCGCCGCGGCGCCATCTCCTCGCCGCGACC
TCCTCCTCGTCCTCCTCCTCCGACCGCTCCTCCGGGTCCAGCAAGTCGGTGAAACCAGCT
GCTGCCGCGCTGCTGACGTCAGCCGCCATCGACCTTTTCTCGCCGGCGCCGGCGCCGACG
ACCCAGCTGCCCGCGTGCCAGCTCTACTACCATAGCCATCCCACGCCGCTGGCACGTGAT
GATCAGCTCATCACCTCGCCGGAGTCGTCGTCGCTCCTCCTGCAGTGGCCGGCGAGCCAG
TACATGCCGGCGACGGAGCTCGGCGGCGTCCTCGGCTCGTCGTCCCACACGCAAACCCCG
GCAGCGATCACCACCCACCCATCGACGATCTCACCCAGCGTGCTCCTCGGCCTATGCAAC
GAGGCACTAGGGCAGCATCAGCAAGAGACCATGGACGACATGATGATCACCTGCTCCAAC
CCCTCCAAGGTGTTCGACCACCATTCCATGGACGACATGAGCTGCACCGACGCGGTGAGC
GCCGTGAACAGGGACGACGAGAAGGCGAGGCTGGGGTTACTGCACTACGGCATCGGCGTC
ACTGCTGCTGCAAATCCGGCACCACATCATCATCATCATCATCATCATCTTGCCTCTCCT
GTGCATGATGCTGTCTCGGCTGCAGATGCTAGTACGGCGGCCATGATCCTTCCATTCACC
ACCACTGCTGCTGCGACGCCGAGCAACGTCGTCGCTACAAGCTCTGCACTCGCTGATCAG
TTGCAAGGGCTGTTGGATGCTGGGTTGCTGCAGGGAGGGGCGGCGCCGCCGCCGCCCTCG
GCGACGGTGGTGGCGGTGAGCCGCGACGACGAGACGATGTGCACCAAGACCACGAGCTAC
AGCTTCCCGGCGACGATGCACCTCAACGTGAAGATGTTCGGCGAGGCGGCCGTGCTGGTG
CGCTACAGCGGCGAGCCGGTGCTCGTCGACGACTCCGGCGTCACCGTCGAGCCGCTGCAG
CAGGGCGCGACCTACTACGTGCTGGTATCTGAGGAAGCTGTGCATTGA
SEQ ID NO: 9 Rice DWL1 = DWARF TILLER-LIKE1 Nucleotide sequence
ATGATGGCCTTAGGCGTGCCACCGCCTCCCTCGCGCGCCTACGTGTCCGGCCCGCTACGC
GACGATGACACTTTTGGCGGTGATCGTGTTCGGCGGCGGCGGCGGTGGCTCAAGGAGCAG
TGCCCTGCCATCATTGTCCACGGGGGTGGCAGGCGTGGAGGGGTCGGCCACAGGGCCCTG
GCTGCCGGAGTCTCTAAAATGCGTCTCCCAGCCCTAAACGCCGCCACCCACCGGATCCCC
TCCACCTCGCCCTTAAGTATCCCTCAGACCCTCACCATCACCCGCGATCCTCCCTACCCA
ATGCTGCCTCGAAGTCACGGCCACCGGACCGGCGGCGGCGGCTTCTCCCTCAAGTCCTCG
CCCTTCTCGTCAGTGGGCGAGGAGAGGGTTCCGGACCCGAAGCCGCGGCGGAACCCGCGG
CCGGAGCAGATCCGGATCCTGGAGGCCATCTTCAACTCCGGCATGGTCAACCCGCCGCGC
GACGAGATCCCGCGCATCCGCATGCAGCTGCAGGAGTACGGCCAGGTCGGCGACGCCAAC
GTCTTCTACTGGTTCCAGAACCGCAAGTCCCGCTCCAAGAACAAGCTGCGCTCCGGCGGG
ACAGGCCGCGCGGGGCTCGGGCTCGGCGGCAACCGGGCCTCAGAGCCGCCGGCGGCGGCG
ACGGCGCACCGGGAGGCCGTGGCACCGTCGTTCACGCCGCCACCGATCCTCCCGCCCCAG
CCGGTGCAGCCGCAGCAGCAGCTTGTCTCGCCGGTGGCGGCGCCCACCTCGTTGTCGTCA
TCGTCCTCCGACCGCTCGTCCGGGTCCAGCAAGCCCGCGAGGGCTACGTTGACGCAGGCG
ATGTCCGTGACGGCGGCCATGGACCTGCTCTCGCCGCTCCGCCGATCAGCTCGGCCACGG
CAAGAGCAGCGCCATGTCTAG
SEQ ID NO: 10 OsBBM1 GenBank accession number: AAX95437.1
MASITNWLGFSSSSFSGAGADPVLPHPPLQEWGSAYEGGGTVAAAGGEETAAPKLEDFLG
MQVQQETAAAAAGHGRGGSSSVVGLSMIKNWLRSQPPPAVVGGEDAMMALAVSTSASPPV
DATVPACISPDGMGSKAADGGGAAEAAAAAAAQRMKAAMDTFGQRTSIYRGVTKHRWTGR
YEAHLWDNSCRREGQTRKGRQVNAGGYDKEEKAARAYDLAALKYWGTTTTTNFPVSNYEK
ELDEMKHMNRQEFVASLRRKSSGFSRGASIYRGVTRHHQHGRWQARIGRVAGNKDLYLGT
FGTQEEAAEAYDIAAIKFRGLNAVTNFDMSRYDVKSIIESSNLPIGTGTTRRLKDSSDHT
DNVMDINVNTEPNNVVSSHFTNGVGNYGSQHYGYNGWSPISMQPIPSQYANGQPRAWLKQ
EQDSSVVTAAQNLHNLHHFSSLGYTHNFFQQSDVPDVTGFVDAPSRSSDSYSFRYNGTNG
FHGLPGGISYAMPVATAVDQGQGIHGYGEDGVAGIDTTHDLYGSRNVYYLSEGSLLADVE
KEGDYGQSVGGNSWVLPTP*
SEQ ID NO: 11
Arabidopsis thaliana BABY BOOM (AtBBM),
NCBI Protein Accession # NP_001332647.1; NP_197245

| | |
|---|---|
| 1 | MNSMNNWLGF SLSPHDQNHH RTDVDSSTTR TAVDVAGGYC FDLAAPSDES |
| 51 | SAVQTSFLSP FGVTLEAFTR DNNSHSRDWD INGGACNNIN NNEQNGPKLE |
| 101 | NFLGRTTTIY NTNETVVDGN GDCGGGDGGG GGSLGLSMIK TWLSNHSVAN |
| 151 | ANHQDNGNGA RGLSLSMNSS TSDSNNYNNN DDVVQEKTIV DVVETTPKKT |
| 201 | IESFGQRTSI YRGVTRHRWT GRYEAHLWDN SCKREGQTRK GRQVYLGGYD |
| 251 | KEEKAARAYD LAALKYWGTT TTTNEPLSEY EKEVEEMKHM TRQEYVASLR |
| 301 | RKSSGESRGA SIYRGVTRHH QHGRWQARIG RVAGNKDLYL GTFGTQEEAA |
| 351 | EAYDIAAIKF RGLSAVTNFD MNRYNVKAIL ESPSLPIGSS AKRLKDVNNP |
| 401 | VPAMMISNNV SESANNVSGW QNTAFQHHQG MDLSLLQQQQ ERYVGYYNGG |
| 451 | NLSTESTRVC FKQEEEQQHF LRNSPSHMTN VDHHSSTSDD SVTVCGNVVS |
| 501 | YGGYQGFAIP VGTSVNYDPF TAAEIAYNAR NHYYYAQHQQ QQQIQQSPGG |
| 551 | DFPVAISNNH SSNMYFHGEG GGEGAPTFSV WNDT |

SEQ ID NO: 12
Brassica napus BABY BOOM1 (BnBBM1)
NCBI Protein Accession # NP_001302749; AAM33802

| | |
|---|---|
| 1 | mnnnwlgfsl spyeqnhhrk dvyssttttv vdvageycyd ptaasdessa iqtsfpspfg |
| 61 | vvvdaftrdn nshsrdwdin gcacnnihnd eqdgpklenf lgrtttiynt nenvgdgsgs |
| 121 | gcygggdggg gslglsmikt wlrnqpvdnv dnqengnaak glslsmnsst scdnnndsnn |
| 181 | nvvaqgktid dsveatpkkt iesfgqrtsi yrgvtrhrwt gryeahlwdn sckregqtrk |
| 241 | grqvylggyd keekaarayd laalkywgtt tttnfpmsey ekeveemkhm trqeyvaslr |
| 301 | rkssgfsrga siyrgvtrhh qhgrwqarig rvagnkdlyl gtfgtqeeaa eaydiaaikf |
| 361 | rgltavtnfd mnrynvkail espslpigsa akrlkeanrp vpsmmmisnn vsesensasg |
| 421 | wqnaavqhhq gvdlsllhqh qeryngyyyn ggnlssesar acfkqeddqh hflsntqslm |
| 481 | tnidhqssvs ddsvtvcgnv vgyggyqgfa apvncdayaa sefdynarnh yyfaqqqqtq |
| 541 | qspggdfpaa mtnnvgsnmy yhgegggeva ptftvwndn |

SEQ ID NO: 13
Brassica napus BABY BOOM2 (BnBBM2)
NCBI Protein Accession # AAM33801; NP_001303138

| | |
|---|---|
| 1 | mnnnwlgfsl spyeqnhhrk dvcsstttta vdvageycyd ptaasdessa iqtsfpspfg |
| 61 | vvldaftrdn nshsrdwdin gsacnnihnd eqdgpklenf lgrtttiynt nenvgdidgs |
| 121 | gcygggdggg gslglsmikt wlrnqpvdnv dnqengngak glslsmnsst scdnnnyssn |
| 181 | nlvaqgktid dsveatpkkt iesfgqrtsi yrgvtrhrwt gryeahlwdn sckregqtrk |
| 241 | grqvylggyd keekaarayd laalkywgtt tttnfpmsey ekeieemkhm trqeyvaslr |
| 301 | rkssgfsrga siyrgvtrhh qhgrwqarig rvagnkdlyl gtfgtqeeaa eaydiaaikf |
| 361 | rgltavtnfd mnrynvkail espslpigsa akrlkeanrp vpsmmmisnn vsesennasg |
| 421 | wqnaavqhhq gvdlsllqqh qeryngyyyn ggnlssesar acfkqeddqh hflsntqslm |
| 481 | tnidhqssvs ddsvtvcgnv vgyggyqgfa apvncdayaa sefdynarnh yyfaqqqqtq |
| 541 | hspggdfpaa mtnnvgsnmy yhgegggeva ptftvwndn |

SEQ ID NO: 14
Oryza sativa BABY BOOM1 (OsBBM1)
NCBI Protein Accession # XP_015616214

| | |
|---|---|
| 1 | masitnwlgf ssssfsgaga dpvlphpplq ewgsayeggg tvaaaggeet aapkledflg |
| 61 | mqvqqetaaa aaghgrggss svvglsmikn wlrsqpppav vggedammal avstsasppv |
| 121 | datvpacisp dgmgskaadg ggaaeaaaaa aaqrmkaamd tfgqrtsiyr gvtkhrwtgr |
| 181 | yeahlwdnsc rregqtrkgr qvylggydke ekaaraydla alkywgtttt tnfpvsnyek |
| 241 | eldemkhmnr qefvaslrrk ssgfsrgasi yrgvtrhhqh grwqarigrv agnkdlylgt |
| 301 | fgtqeeaaea ydiaaikfrg lnavtnfdms rydvksiies snlpigtgtt rrlkdssdht |
| 361 | dnvmdinvnt epnnvvsshf tngvgnygsq hygyngwspi smqpipsqya ngqprawlkq |
| 421 | eqdssvvtaa qnlhnlhhfs slgythnffq qsdvpdvtgf vdapsrssds ysfryngtng |
| 481 | fhglpggisy ampvatavdq gqgihgyged gvagidtthd lygsrnvyyl segslladve |
| 541 | kegdygqsvg gnswvlptp |

SEQ ID NO: 15
Oryza sativa BABY BOOM (OsBBM)
NCBI Protein Accession # XP_015634444

| | |
|---|---|
| 1 | matmnnwlaf slspqdqlpp sqtnstlisa aattttagds stgdvcfnip qdwsmrgsel |
| 61 | salvaepkle dflggisfse qqhhhggkgg vipssaaacy assgssvgyl ypppsssslq |
| 121 | fadsvmvats spvvahdgvs gggmvsaaaa aaasgnggig lsmiknwlrs qpapqpaqal |
| 181 | slsmnmagtt taqgggamal lagagergrt tpaseslsts ahgattatma ggrkeineeg |
| 241 | sgsagavvav gsesggsgav veagaaaaaa rksvdtfgqr tsiyrgvtrh rwtgryeahl |
| 301 | wdnscrregq trkgrqvylg gydkeekaar aydlaalkyw gpttttnfpv nnyekeleem |
| 361 | khmtrqefva slrrkssgfs rgasiyrgvt rhhqhgrwqa rigrvagnkd lylgtfstqe |
| 421 | eaaeaydiaa ikfrglnavt nfdmsrydvk sildsaalpv gtaakrlkda eaaaaydvgr |
| 481 | iashlggdga yaahyghhhh saaaawptia fqaaaappph aaglyhpyaq plrgwckqeq |
| 541 | dhaviaaahs lqdlhhlnlg aaaaahdffs qamqqqhglg sidnaslehs tgsnsvvyng |
| 601 | dnggggggyi mapmsavsat atavasshdh ggdggkqvqm gydsylvgad ayggggagrm |
| 661 | pswamtpasa paatsssdmt gvchgaqlfs vwndt |

SEQ ID NO: 16
Zea mays BABY BOOM1 (ZmBBM1) GRMZM2G366434
NCBI Protein Accession # NP_001147535

| | |
|---|---|
| 1 | masannwlgf slsgqdnpqp nqdsspaagi disgasdfyg lptqqgsdgh lgvpglrddh |
| 61 | asygimeayn rvpqetqdwn mrgldynggg selsmlvgss gggggngkra vedsepkled |
| 121 | flggnsfvsd qdqsggylfs gvpiassans nsgsntmels miktwlrnnq vaqpqppaph |
| 181 | qpqpeemstd asgssfgcsd smgrnsmvaa ggssqslals mstgshlpmv vpsgaasgaa |
| 241 | sestssenkr asgamdspgs aveavprksi dtfgqrtsiy rgvtrhrwtg ryeahlwdns |
| 301 | crregqsrkg rqvylggydk edkaaraydl aalkywgttt ttnfpisnye keleemkhmt |
| 361 | rqeyiaylrr nssgfsrgas kyrgvtrhhq hgrwqarigr vagnkdlylg tfsteeeaae |
| 421 | aydiaaikfr glnavtnfdm srydvksile sstlpvggaa rrlkdavdhv eagatiwrad |
| 481 | mdgavisqla eagmggyasy ghhgwptiaf qqpsplsvhy pygqpsrgwc kpeqdaaaaa |
| 541 | ahslqdlqql hlgsaahnff qasssstvyn ggagasggyq glgggssflm psstvvaaad |
| 601 | qghsstanqg stcsygddhq egkligydaa mvataaggdp yaaarngyqf sqgsgstvsi |
| 661 | arangyannw sspfnngmg |

SEQ ID NO: 17
Glycine max BABY BOOM1 (GmBBM1)
NCBI Protein Accession # XP_006586645.1; ADP37371

| | |
|---|---|
| 1 | mgsmnllgfs lspheehpss qdhsqttpsr fsfnpdgsis stdvaggcfd ltsdstphll |
| 61 | nlpsygiyea fhrnnsintt qdwkenynsq nlllgtscnk qnmnqnqqqq pklenflggh |
| 121 | sfgeheqtyg gnsastdymf paqpvsaggg gsgggsnnnn nsnsiglsmi ktwlrnqppn |
| 181 | seninnnnes ggnirssvqq tlslsmstgs qsstslpllt asvdngesps dnkqpntsaa |
| 241 | ldstqtgaie taprksidtf gqrtsiyrgv trhrwtgrye ahlwdnscrr egqtrkgrqv |
| 301 | ylggydkeek aaraydlaal kywgtttttn fpishyekel eemkhmtrqe yvaslrrkss |
| 361 | gfsrgasiyr gvtrhhqhgr wqarigrvag nkdlylgtfs tqeeaaeayd vaaikfrgls |
| 421 | avtnfdmsry dvksilestt lpiggaakrl kdmeqvelsv dnghradqvd hsiimsshlt |
| 481 | qginnnyagg gtathhnwhn ahafhqpqpc ttmhypygqr inwckqeqqd nsdaphslsy |
| 541 | sdihqlqlgn ngthnffhtn sglhpmlsmd sasidnssss nsvvydgygg gggynvmpmg |
| 601 | tttavvasdg dqnprsnhgf gdneikalgy esvygsatds yhaharnlyy ltqqqsssvd |
| 661 | tvkasaydqg sacntwvpta ipthaprstt smalchgatt pfsllhe |

SEQ ID NO: 18
Capsicum annuum BABY BOOM (CaBBM)
NCBI Protein Accession # XP_016568915

| | |
|---|---|
| 1 | mkmksmndds sssnnsnsnn nnhssaatns nnwlgfslsp hmkmevtnas etqqqqhphq |
| 61 | qqfaqsfyls sspttmnvst asalcyennp fhsslsvmpl ksdgslcime alsrshadam |
| 121 | vqssspkled flggasqygs hereamalsl dslyyhqnde diqvhshhpy yspmhchgmy |
| 181 | qeslleetkp tqisncdaqm tgnemkswgh yaidqhindt csmvaaaaav aagggggtvg |
| 241 | cndlqslsls mnpgtqsscv tprqisptgl ecvaieskkr asakvaqkqp vhrksidtfg |
| 301 | qrtsqyrgvt rhrwtgryea hlwdnsckke gqtrkgrqvy lggydmedka araydqaalk |
| 361 | ywgpsthinf plenyqkele emknmtrqey vahlrrkssg fsrgasiyrg vtrhhqhgrw |
| 421 | qarigrvagn kdlylgtfst qeeaaeaydv aaikfrgvna vtnfdisryd vekimasnnl |
| 481 | pagelarrtk erepresiey nnisvhknee cvqnnnnngn itdwkmvlyq asnpsigsnn |
| 541 | yrnpsfsval qdligidsin nstshatild heqnkiganh fsnasslvts lgssreaspd |
| 601 | ktaaslvfak ptkfvvpttn vnacipsaql rpipvsmahl pvfaalnda |

SEQ ID NO: 19
Medicago truncatula BABY BOOM (MtBBM),
NCBI Protein Accession # XP_003624212

| | |
|---|---|
| 1 | masmnllgfs lspqeqhpst qdqtvasrfg fnpneisgsd vqgdhcydls shttphhsln |
| 61 | lshpfsiyea fhtnnnihtt qdwkenynnq nlllgtscmn qnvnnnnqqa qpklenflgg |
| 121 | hsftdhqeyg gsnsysslhl pphqpeascg ggdgstsnnn siglsmiktw lrnqppppen |
| 181 | nnnnnnesga rvqtlslsms tgsqssssvp llnanvmsge isssenkqpp ttavvldsnq |
| 241 | tsvvesavpr ksvdtfgqrt siyrgvtrhr wtgryeahlw dnscrregqt rkgrqvylgg |
| 301 | ydkeekaara ydlaalkywg tttttnfpis hyekeveemk hmtrqeyvas lrrkssgfsr |
| 361 | gasiyrgvtr hhqhgrwqar igrvagnkdl ylgtfstqee aaeaydvaai kfrglsavtn |
| 421 | fdmsrydvkt ilesstlpig gaakrlkdme qvelnhvnvd ishrteqdhs iinntshlte |
| 481 | qaiyaatnas nwhalsfqhq qphhhynann mqlqnypygt qtqklwckqe qdsddhstyt |
| 541 | tatdihqlql gnnnnnthnf fglqnimsmd sasmdnssgs nsvvygggdh ggyggnggym |
| 601 | ipmaiandgn qnprsnnnfg eseikgfgye nvfgtttdpy haqaarnlyy qpqqlsvdqg |
| 661 | snwvptaipt laprttnvsl cppftllhe |

SEQ ID NO: 20
Cenchrus ciliaris CcASGR-BBM-like1
NCBI Protein Accession # ACD80125

| | |
|---|---|
| 1 | mgstnnwlrf vsfsggggak daaallplpp sprgdvdeag aepkledflg lqepsaaavg |
| 61 | agrpfavggg assiglsmik nwlrsqpapa gpaagvdsmv laaaaastev agdgaeggga |
| 121 | vadavqqrka aavdtfgqrt siyrgvtkhr wtgryeahlw dnscrregqt rkgrqvylgg |
| 181 | ydkeekaara ydlaalkyrg tttttnfpms nyekeleemk hmsrqeyvas lrrkssgfsr |
| 241 | gasiyrgvtr hhqhgrwqar igsvagnkdl ylgtfstqee aaeaydiaai kfrglnavtn |
| 301 | fdmsrydvks iiessslpvg gapkrlkevp dqsdmginin gdsaghmtai nlltdgndsy |
| 361 | gaesygysgw cptamtpipf qfsighdhsr lwckpeqdna vvaalhnlhh lqhlpapvgt |
| 421 | hnffqpspvq dmtgvadass ppvesnsfly ngdvgyhgam ggsyampvat lvegnsagsg |
| 481 | ygveegtgse ifggrnlysl sqgssgantg kadayeswdp smlvisqksa nvtvchgapv |
| 541 | fsvwk |

SEQ ID NO: 21
Pennisetum squamulatum PsASGR-BBM-like1
NCBI Protein Accession # ACD80127

| | |
|---|---|
| 1 | mgstnnwlrf asfsggggak daaallplpp sprgdvdeag aepkledflg lqepsaaavg |
| 61 | agrpfavggg assiglsmir nwlrsqpapa gpaagvdsmv laaaaastev agdgaeggga |
| 121 | vadavqqrka aavdtfgqrt siyrgvtkhr wtgryeahlw dnscrregqt rkgrqggydk |
| 181 | eekaaraydl aalkyrgttt ttnfpmsnye keleemkhms rqeyvaslrr kssgfsrgas |
| 241 | iyrgvtrhhq hgrwqarigs vagnkdlylg tfstqeeaae aydiaaikfr glnavtnfdm |
| 301 | srydvksiie ssslpvggtp krlkevpdqs dmginingds aghmtainll tdgndsygae |
| 361 | sygysgwcpt amtpipfqfs nghdhsrlwc kpeqdnavva alhnlhhlqh lpapvgthnf |
| 421 | fqpspvqdmt gvadassppv esnsflyngd vgyhgamggs yampvatlve gnsagsgygv |
| 481 | eegtgseifg grnlyslsqg ssgantgkad ayeswdpsml visqksanvt vchgapvfsv |
| 541 | wk |

SEQ ID NO: 22
Pennisetum squamulatum PsASGR-BBM-like2
NCBI Protein Accession # ACD80124.2

| | |
|---|---|
| 1 | mgstnnwlrf asfsggggak daaallplpp sprgdvdeag aepkledflg lqepsaaavg |
| 61 | agrpfavggg assiglsmir nwlrsqpapa gpaagvdsmv laaaaastev agdgaeggga |
| 121 | vadavqqrka aavdtfgqrt siyrgvtkhr wtgryeahlw dnscrregqt rkgrqvylgg |
| 181 | ydkeekaara ydlaalkyrg tttttnfpms nyekeleemk hmsrqeyvas lrrkssgfsr |
| 241 | gasiyrgvtr hhqhgrwqar igsvagnkdl ylgtfstqee aaeaydiaai kfrglnavtn |
| 301 | fdmsrydvks iiessslpvg gtpkrlkevp dqsdmginin gdsaghmtai nlltdgndsy |
| 361 | gaesygysgw cptamtpipf qfsnghdhsr lwckpeqdna vvaalhnlhh lqhlpapvgt |
| 421 | hnffqpspvq dmtgvadass ppvesnsfly ngdvgyhgam ggsyampvat lvegnsagsg |
| 481 | ygveegtgse ifggrnlysl sqgssgantg kadayeswdp smlvisqksa nvtvchgapv |
| 541 | fsvwk |

SEQ ID NO: 23
Rosa canina BABY BOOM1 (RcBBM1)
NCBI Protein Accession # AGZ02154.1

| | |
|---|---|
| 1 | mnmnwlgfsl spqeddhapi shqladqetl asrlgfnsne iqgagggtdv sgggssecfd |
| 61 | vtnsdstasv nhhltpsifg iheaaafnrn ndhihsqdwn mkgagmnssd snnykrtsss |
| 121 | dlstmlmgsn tsttstsysi isqqanlenh hqqlpklenf lgrhsfadhd sssaghdymf |
| 181 | dmnngpgpvs snvmntktns nniglsmikt wlrnqpsqpr dnhhqleqes knskesrnqp |
| 241 | qsslslsmgt gslqtvttat aggaatgett sssdkntkqs pvvtattttg tdaqtqstgg |
| 301 | aieavprkai dtfgqrtsiy rgvtrhrwtg ryeahlwdns crregqtrkg rqvylggydk |
| 361 | edkaaraydl aalkywgttt ttnfpissye keidemkpmt rqeyvaslrr kssgfsrgas |
| 421 | iyrgvtrhhq hgrwqarigr vagnkdlylg tfstqeeaae aydiaaikfr glnavtnfdm |
| 481 | srydvksile ssalpittga takrlkdvqq qqppppadhh hqimlssvld hhgqvirsss |
| 541 | stehdimsnv ysaygsygaq qgcswptlaf nqaqaqaaaa phqapfaagi ngmqlhyspy |
| 601 | gygygnahaq rvwckqeqdt nsnqersfhh qdddhlrqql qlggthnffh dhdqqqqqqq |
| 661 | qqtsglmglm dssaasmehs sgsnsviysg gdhhgnnngy gssttgtggg yimpmvmstv |
| 721 | vanddqnqad gnnnningfg dgddqeanik aqqlgydhhp qnmflgssst idpayqhhas |
| 781 | nrnlyyhlpv qddqhesssv avatsstton mnwvptavpt lahptftvwn dt |

SEQ ID NO: 24
Rosa canina BABY BOOM2 (RcBBM2)
NCBI Protein Accession # AGZ02155.1

| | |
|---|---|
| 1 | mnmnwlgfsl spqeddhapi shqladqetl asrlgfnsne ihgagggtdv sgggssecfd |
| 61 | ltnsdstasv nhhltpsifg iheaaafnrn ndhihsqdwn mkgagmnssd snnykrtsss |
| 121 | dlstmlmgsn tttstscsii sqqanlenhh qqlpklenfl grhsfadhdr ssaghdymfd |
| 181 | mnngpgpvss nvmntktnsn niglsmiktw lrnqpsqprd nhhqleqesk nskesrnqpq |
| 241 | sslslsmgtg slqtvttata ggaataatge ttcssdkntk qspvvtattt tgtdaqtqst |
| 301 | ggaieavprk aidtfgqrts iyrgvtrhrw tgryeahlwd nscrregqtr kgrqvylggy |
| 361 | dkedkaaray dlaalkywgt ttttnfpiss yekeidemkp mtrqeyvasl rrkssgfsrg |
| 421 | asiyrgvtrh hqhgrwqari grvagnkdly lgtfstqeea aeaydiaaik frglnavtnf |
| 481 | dmsrydvksi lessalpitt gatakrlkev qqqqppppad hhhqimlssv ldhhgqiirs |
| 541 | ssstehdims nvysaygsyg aqqgcswptl afnqaqaqaa aaphqapfaa gingmqlhys |
| 601 | pygygygnah aqrvwckqeq dtnsnqessf hhqdddhlrq qlqlggthnf fhdhdqqqts |
| 661 | glmglmdssa asmehssgsn sviysggdhh gnnngygsst tgtgggyimp mvmstvvand |
| 721 | dqnqadgnnn ningfedgdd qeanikaqql gydhhhqnmf lgssstidpa yqhhasnrnl |
| 781 | yyhlpvqddq hesssvavat ssttcnmnwv ptavptlahp tftvwndt |

SEQ ID NO: 25
Ricinus communis AIL6
NCBI Protein Accession # XP_015583464

| | |
|---|---|
| 1 | mapattnwls fslspmemlr sstesqfisy egsstatpsp hyfidnfyan gwgnpkeaqg |
| 61 | attmaaetsi ltsfidpeth hqqvpkledf lgdsssivry sdnsqtdtqd sslthiydqg |
| 121 | saayfseqqd lkaiagfqaf stnsgsevdd sasiarthlg gefmghsids sgndqlggfs |
| 181 | nctaannals lavnnnnnnn gnqsatnskt iapviesdcp kkiadtfgqr tsiyrgvtrh |
| 241 | rwtgryeahl wdnscrregq arkgrqgalf flfspsssyh lslfvacffn yssvkilgiy |
| 301 | axsaiphghv lyffqvtnyt keldemkyvs kqefiaslrr kssgfsrgas iyrgvtrhhq |
| 361 | qgrwqarigr vagnkdlylg tfateeeaae aydiaaikfr gmnavtnfem srydveaimk |
| 421 | salpiggaak rlklsleseq kpnlnheqqp qgsssnsssn nisfasmppv taipcgipfe |
| 481 | nttaqlyhhh hhhhhhqhhn lfhhlqttnn nlggttdiss gsttssmatt msmlpqtaef |
| 541 | flwphhqsy |

SEQ ID NO: 26
Elaeis guineensis EgAP2-1
NCBI Protein Accession # AAV98627; NP_001290493

| | |
|---|---|
| 1 | mdmdtshswl afslsyhqpy llealssapp hggggmtaee rggsaevaam avvgpkledf |
| 61 | lggcgepmgr yaggetgdag giydselkhi aagylqglpa teqqdsemak vaapaesrka |
| 121 | vetfgqrtsi yrgvtrhrwt gryeahlwdn scrregqsrk grqvylggyd keekaarayd |
| 181 | laalkywgpt tttnfpisny ekeleemknm trqefvaslr rkssgfsrga siyrgvtrhh |
| 241 | qhgrwqarig rvagnkdlyl gtfstqeeaa eaydiaaikf rglnavtnfd isrydvksia |
| 301 | nsnlpiggmt grpskatess pssssdamtv eakqlldgrd psaslgfaal pikhdqdfws |
| 361 | lfalqqqqqq qqqqsnqasg fglfssgvtm dfstasngvi sqgcggslvw nggvvgqqqe |
| 421 | qsqnnscssi pyatpiafgg nyegssyvgs wvtpppsyyh epakpnvavf qtpifgme |

SEQ ID NO: 27
Populus trichocarpa BABY BOOM1 (PtBBM1)
NCBI Protein Accession # XP_002316179

| | |
|---|---|
| 1 | masmnnwlgf slshqelpss qsdhhqdhsq ntdsrlgfhs deisgtnvsg ecfdltsdst |
| 61 | apslnlpatf gileafrnnq pqdwnmkslg mnpdtnykta sglpifmgts cnsqtidqnq |
| 121 | epklenflgg hsfgnhehkl ngcntmydtt gdyvfqncsl qlpseatsne rtsnngggdn |
| 181 | knssiglsmi ktwlrnqpap tqqdtnnknn ggaqslslsm stgsqsaasa lpllavnggv |
| 241 | nntggdqsss dnnkqqkstt psldsqtgai esvprksidt fgqrtsiyrg vtrhrwtgry |
| 301 | eahlwdnscr regqtrkgrq vylggydkee kaaraydlaa lkywgttttt nfpitnyeke |
| 361 | ieemkhmtrq eyvaslrrks sgfsrgasiy rgvtrhhqhg rwqarigrva gnkdlylgtf |
| 421 | stqeeaaeay diaaikfrgl navtnfdmsr ydvnsiless tlpiggaakr lkeaehaeia |
| 481 | mdiaqrtddh dnmgsqltdg issygavqhg wptvafqqaq pfsmhypygq rlwckqeqds |
| 541 | dnrsfqelhq lqlgnthnff qpsvlhnlvs mdsssmehss gsnsvvyssg vndgtstgtn |
| 601 | ggyqgigygs sagyavpmat visnndnnhn qgngygdgdq vkalgyenmf spsdpyharn |
| 661 | lhylsqqpsa ggikasaydq gsacynwvpt avptiaaars nnmavchgaq pftvwndgt |

SEQ ID NO: 28
Populus trichocarpa BABY BOOM2 (PtBBM2)
NCBI Protein Accession # XP_002311259

| | |
|---|---|
| 1 | mastnnwlgf slspqelpss qsdhhdhpqn tdsrlrfhsd eisgtdvsge sfdltsdsta |
| 61 | pslnlpasfg ileafrnnqs qdwnnmkrsg inedtsyntt sdvpifmgss cnsqnidqnq |
| 121 | epklenflgg hsfgnhehkl nvcstmygst ghymfhncsl qlpsedasne rtssnggadt |
| 181 | sinnnntnss iglsmiktwl knqpaptqqd tnnksnggaq slslsmstgs qsgsdlplla |
| 241 | vngggnrtrg eqsssdnnkq qkttpsldsq tgaievvprk sidtfgqrts iyrgvtrhrw |
| 301 | tgryeahlwd nscrregqtr kgrqggydke dkaaraydla alkywgtttt tnfpmsnyek |
| 361 | eieemkhmtr qehvaslrrk ssgfsrgasi yrgvtrhhqh grwqarigrv agnkdlylgt |
| 421 | fstqeeaaea ydiaaikfrg lnavtnfdmn rydvnsimes stlpiggaak rlkeaehaei |
| 481 | ttrvqrtddh dstssqltdg isnygtaahh gwptiafqqa qaftmhypyg qrlwckqeqd |
| 541 | sdnhsfqelh qlqlgntqnf lqpsvlhnvm smesssmehs sgsdsvmyss gghdgtgtgt |
| 601 | ngsyqgigyg sntgyaipma tviandvntq dqgngygdge vkalgyenmf sssdpyharn |
| 661 | lyylsqqssa gvikasaydq gstcnnwlpt avptiaarsn nmavchgapt ftvwnest |

SEQ ID NO: 29
Larix gmelinii var. olgensis x Larix kaempferi BABY BOOM (BBM) ; KJ004517
NCBI Protein Accession # AHH34920.1

| | |
|---|---|
| 1 | mgstsnwlaf slsphltvdm pdstqprsts aasnhsrhhn dfsngtvhdc yelhptdtmq |
| 61 | mplrpdgslc ilealdrtqn nqdwqlksle npgsmdlesd vqsqqmmkse lsilaggsse |
| 121 | qmsasigrhk nvdqegpkle dflggaslrg hyndartdsi ygnddafdek mmapglrdvv |
| 181 | pnclngfdvt dtelssgskk tdqnqdstrn insiqnslvq dsydqnsndq ymfqdcslql |
| 241 | ppnsgannmi glsmiktwlr sqpcpenkmn aatnsstpts akdqslgnlt niqslslsms |
| 301 | pgsqssspla lpvqyqntna dspsseskkr slekqslvsv eatprksidt fgqrtsiyrg |
| 361 | vtrhrwtgry eahlwdnscr regqtrkgrq vylggydkee kaaraydlaa lkywgptttt |
| 421 | nfptgnyeke leemkhmtrq eyvaslrrks sgfsrgasiy rgvtrhhqhg rwqarigrva |
| 481 | gnkdlylgtf ssqeeaaeay diaaikfrgl navtnfdmtr ydvnsiless tlpiggaaak |
| 541 | rikdaepsdp svdgrrtdde isstissqia dtltsygnaa ypnghagwpi iafqqqtnph |
| 601 | apafysqqra aagwckqehn niqnhdlqlh fqsstqnflq psmmtsnant vlhnlmnles |
| 661 | saqldgtntn snsglfsnis gnlagnslqm anspipsgit vcdsartpfs tendgsstkn |
| 721 | ssyndnmlsn sdpfarglyy lsqhspsvvk anyenaaynn wmtpavqtla prpnltvcha |
| 781 | piftvwndt |

SEQ ID NO: 30: Arabidopsis DD45 promoter
CCTCTTTGTCACCGTCACTCTTCTCCTCGTTCTCAACGTCTCCAGCAGAGCACTCCCGCCCGTGGCGGATTCCAC
CAACATAGCGGCTAGACTAACCGGAGGAGGACTGATGCAGTGTTGGGATGCACTCTACGAGCTGAAGTCATGTAC
TAATGAGATCGTTCTCTTCTTTCTCAACGGTGAGACCAAACTCGGCTACGGTTGCTGCAACGCCGTTGATGTCAT
TACCACTGATTGTTGGCCGGCGATGCTTACTTCTCTTGGCTTTACACTGGAGGAAACCAATGTCCTCCGTGGTTT
CTGTCAATCTCCGAACTCCGGCGGTTCTTCTCCAGCTCTTTCCCCTGTCAAACTTTGATAAATGTTCCTCGCTGA
CGTAAGAAGACATTAGTAATGGTTATAATATATAGCTTTCTATGAATGTATGGTGAGAAAATGTCTGTTCACTGA
TTTTGAGTTTG
GAATAAAAGCATTTGCGTTTGGTTTATCATTGCGTTTATACAAGGACAGAGATCCACTGAGCTGGAATAGCTTAA
AACCATTATCAGAACAAAATAAACCATTTTTTGTTAAGAATCAGAGCATAGTAAACAACAGAAACAACCTAAGAG
AGGTAACTTGTCCAAGAAGATAGCTAATTATATCTATTTTATAAAAGTTATCATAGTTTGTAAGTCACAAAAGAT
GCAAATAACAGAGAAACTAGGAGACTTGAGAATATACATTCTTGTATATTTGTATTCGAGATTGTGAAAATTTGA
CCATAAGTTTAAATTCTTAAAAAGATATATCTGATCTAGATGATGGTTATAGACTGTAATTTTACCACATGTTTA
ATGATGGATAGTGACACACATGACACATCGACAACACTATAGCATCTTATTTAGATTACAACATGAAATTTTTCT
GTAATACATGTCTTTGTACATAATTTAAAAGTAATTCCTAAGAAATATATTTATACAAGGAGTTTAAAGAAAACA
TAGCATAAAGTTCAATGAGTAGTAAAAACCATATACAGTATATAGCATAAAGTTCAATGAGTTTATTACAAAAGC
ATTGGTTCACTTTCTGTAACACGACGTTAAACCTTCGTCTCCAATAGGAGCGCTACTGATTCAACATGCCAATAT
ATACTAAATACGTTTCTACAGTCAAATGCTTTAACGTTTCATGATTAAGTGACTATTTACCGTCAATCCTTTCCC
ATTCCTCCCACTAATCCAACTTTTTAATTACTCTTAAATCACCACTAAGCTTCGAATCCATCCAAAACCACAATA
TAAAAACAGAACTCTCGTAACTCAATCATCGCAAAACAAAACAAAACAAAACAAAAACCCCAAAAAGAAAGAATA
SEQ ID NO: 31: Rice egg cell-specific promoter sequence from LOC_Os03g18530 OsECA1
gene
ATGGAATGATGGATGAATGTTCACGTTCTTGAGTTCCTAAATGGTACTAATTTTGCAAAAACTTTCTATATGTGT
TTTTTGTTAAGAATGTTGTTTTAAACCCATCTTTTCACTTTATAATATTTAATTAAATCGTTCGTACCCTCGAAT
AGTTATTGCAAATTATACTTAACTATTCAGTCATTCAGCACAAAAGAACAGGGCCATGAAATTGTAATACTAGTA
CATTTCTGTTCTTTTCTTTTCTTTTTGAGGTTGTCTGAAACACCTGTATCTTAAACTATCGCAGACTAGCCAATG
AGTCGTACTCACCTGAAACTGAAACCAAGTGATTAACCAAGCTGGTTCGACAGTAATTCCATCCATAATGCAGCT
CCGGAGCCCTTCATATCCTGCATGTTACTCAAACAACATCCCCACCTCCTCATTTCCTCTCCCCTATTGCATTGC
ATAATTGCAGAAGATTAAGCCGCTAATGCATAATTACACATTATTTGTGTCCACTAATTTTCCCTTTCCCACACG
CTACGAAACTCAAAAGCCGGCCTCCTCGCCTCCTTCCCTGAACGTTACTAATCGCGTCATGTATAAATACAGAGC
TTGCCCACGCACCGGCACATTGCATCGCACTACGCACATCTACACGATACCCAAGCAGCAAAGCTAGAAAGAAAA
ACC
SEQ ID NO: 32: ARABIDOPSIS EGG CELL 1.1 (EC1.1) PROMOTER SEQUENCES FROM
AT1G76750 GENE
GTTGCCTTATGATTTCTTCGGTTTCAAGATGATCAAATAGTTATAGATTTCATGCT
CACACATGCTCATTAGATGTGTACATACTTTACTTACCCAAATCTATTTTCTCGCA
AAGATTTTGATGGTAAAGCTGATTTGGTTCTATTGAACTAAATCAAACGAGTTTC
AGACTGAGTGATTCTAATCCGGCCCATTAGCCCCTAAACAGACCCACTAATTACG
CAGCTTTTAATAGAGTAATTACACCTAGTTTACCCACTAAACCACTAAGCACTAA
TTATCTCACAATCTAATGAGCTTCCCTCGTAATTACTTGGGCTTTCACTCTACCAT
TTATTTGTAACAGTCAAGTCTCTACTGTCTCTATATAAACTCTCTAAAGTTAACAC
ACAATTCTCATCACAAACAAATCAACCAAAGCAACTTCTACTCTTTCTTCTTTCG
ACCTTATCAATCTGTTGAGAA
SEQ ID NO: 33: DWT conserved central domain from rice:
PDPKPRWNPRPEQIRILEAIFNSGMVNPPRDEIPRIRMQLQEYGQVGDANVFYWFQNRKSRSKNKLR
SEQ ID NO: 34
Rice OSD1; Locus# LOC_Os02g37850 CDS Sequence
Os02g37850.2
atgcctgaagtgagaaattccggcggtagggcggcgctcgccgacccctcgggtggtggg
ttctttatcaggaggacgacgtcgccgccgggagccgtggcggtcaagccgctggctcgg
cgggccctgccgccgacgagcaacaaggagaacgtgccgccgtcctgggctgtgaccgtg
agggctacacccaagaggaggagccccctgcccgagtggtacccgaggagcccactccgc
gacatcacgtcagtcgtcaaggcagttgagaggaaaagtcgcctcggaaatgctgcggtt
cggcagcagatccagttgagtgaagattcttcacgatctgtggatccagcaactccagta
caaaaagaagaaggtgtccctcaaagcacaccaacaccaccaactcaaaaggccctggat
gctgctgccccttgtcctggctcaacccaagctgttgcaagcacatcaacagcttacttg
gccgagggcaagccgaaggcatcatcttcttctccatctgactgctcctttcagacacca
tccagaccaaatgatccagctcttgctgatctcatggagaaggaactgtccagctccata
gagcagatagagaagatggtaaggaagaacctcaagagagctccgaaggctgctcagcct
tccaaggtgaccatccagaagcgcaccctgttgtccatgagatga
SEQ ID NO: 35
Rice OSD1; Locus# LOC_Os02g37850 Protein Sequence
MPEVRNSGGRAALADPSGGGFFIRRTTSPPGAVAVKPLARRALPPTSNKENVPPSWAVTV
RATPKRRSPLPEWYPRSPLRDITSVVKAVERKSRLGNAAVROQIQLSEDSSRSVDPATPV
QKEEGVPQSTPTPPTQKALDAAAPCPGSTQAVASTSTAYLAEGKPKASSSSPSDCSFQTP
SRPNDPALADLMEKELSSSIEQIEKMVRKNLKRAPKAAQPSKVTIQKRTLLSMR*
SEQ ID NO: 36
Arabidopsis OSD1; Locus#AT3G57860 CDS Sequence
AT3G57860.1

| | |
|---|---|
| 1 | ATGCCAGAAG CAAGAGATCG AACCGAGAGG CCTGTGGATT ACTCGACTAT |
| 51 | ATTTGCCAAC CGACGGAGAC ATGGTATTTT ACTTGACGAG CCAGATTCAC |
| 101 | GGCTTAGTTT GATTGAATCT CCGGTGAATC CAGATATTGG GTCTATTGGT |
| 151 | GGAACGGGCG GGCTTGTGAG AGGCAATTTC ACTACATGGA GGCCTGGTAA |
| 201 | TGGCAGAGGT GGTCACACTC CATTTAGATT GCCACAGGGA AGAGAGAATA |
| 251 | TGCCCATAGT GACCGCTAGG CGTGGAAGAG GTGGTGGTTT GTTGCCTTCT |
| 301 | TGGTATCCAA GAACACCICT ACGCGACATA ACTCATATTG TGCGGGCTAT |
| 351 | TGAGAGAAGA AGAGGAGCTG GGACTGGAGG AGACGATGGC CGAGTTATTG |
| 401 | AGATCCCAAC TCATCGACAA GTTGGTGTTC TTGAATCTCC AGTACCACTG |
| 451 | TCAGGAGAAC ACAAATGCTC GATGGTCACT CCTGGACCAT CTGTGGGATT |
| 501 | CAAGCGTAGT TGCCCACCAT CAACTGCTAA AGTTCAAAAG ATGTTACTTG |
| 551 | ACATCACTAA AGAGATAGCT GAGGAAGAAG CTGGCTTCAT CACACCCGAG |
| 601 | AAGAAGCTAC TCAATTCTAT TGACAAAGTT GAGAAAATTG TGATGGCGGA |
| 651 | GATCCAGAAG TTGAAGAGCA CTCCTCAAGC TAAAAGGGAA GAGCGGGAGA |
| 701 | AGAGGGTGCG GACTTTAATG ACTATGCGAT GA |

SEQ ID NO: 37
Arabidopsis OSD1; Locus#AT3G57860 Protein Sequence

| | |
|---|---|
| 1 | MPEARDRTER PVDYSTIFAN RRRHGILLDE PDSRESLIES PVNPDIGSIG |
| 51 | GIGGLVRGNF TTWRPGNGRG GHTPFRLPQG RENMPIVTAR RGRGGGLLPS |
| 101 | WYPRTPLRDI THIVRATERR RGAGTGGDDG RVIEIPTHRQ VGVLESPVPL |
| 151 | SGEHKCSMVT PGPSVGFKRS CPPSTAKVQK MLLDITKEIA EEEAGFITPE |
| 201 | KKLLNSIDKV EKIVMAEIQK LKSTPQAKRE EREKRVRTLM TMR |

SEQ ID NO: 38
Rice PAIR1, Locus# LOC_Os03g01590 CDS Sequence
>LOC_Os03g01590.1
ATGAAGCTTAAGATGAACAAGGCCTGCGACATCGCCTCCATCTCCGTCCTCCCTCCCCGG
AGGACCGGAGGGAGCAGCGGCGCGTCGGCTTCCGGTTCCGTGGCGGTGGCGGTGGCGTCT
CAGCCGCGGTCGCAGCCGCTCTCGCAGTCGCAGCAGTCCTTCTCGCAGGGCGCCTCCGCC
TCGCTCTTGCACTCGCAGTCGCAGTTCTCGCAGGTCTCCCTCGACGACAACCTCCTCACC
CTCCTCCCTTCCCCCACCCGCGATCAGAGATTTGGCTTGCATGATGACTCATCCAAGAGG
ATGTCCTCTTTACCAGCCAGTTCAGCTTCTTGCGCGCGAGAAGAGTCTCAGCTGCAACTG
GCAAAATTACCAAGCAACCCAGTGCACCGCTGGAACCCCTCCATTGCAGATACTAGATCA
GGTCAGGTTACTAATGAGGATGTTGAGCGCAAATTTCAGCATCTGGCAAGCTCAGTACAT
AAGATGGGGATGGTGGTAGACTCAGTCCAAAGTGACGTAATGCAGTTAAACAGAGCCATG
AAGGAGGCATCATTAGATTCTGGTAGCATACGGCAAAAGATTGCTGTCCTTGAAAGCTCA
CTTCAGCAAATTCTTAAGGGACAAGACGATCTCAAAGCACTCTTTGGAAGCAGCACAAAA
CACAATCCTGATCAGACAAGTGTTCTGAATTCTCTAGGCAGCAAATTGAATGAGATATCC
TCGACCCTTGCAACCTTGCAGACACAAATGCAAGCAAGACAACTGCAGGGTGATCAGACA
ACTGTTCTGAATTCTAATGCCAGCAAATCGAATGAGATATCCTCGACTCTTGCAACCCTG
CAGACACAAATGCAAGCAGATATAAGACAACTGCGGTGTGACGTCTTCAGAGTTTTTACA
AAAGAGATGGAGGGGGTTGTTAGAGCTATCAGGTCTGTCAATAGTAGGCCTGCTGCAATG
CAAATGATGGCAGACCAGAGTTACCAAGTACCAGTTTCAAATGGATGGACCCAGATTAAC
CAGACACCAGTAGCAGCTGGAAGGTCTCCAATGAACCGAGCACCAGTAGCAGCTGGAAGG
TCCCGGATGAACCAATTACCTGAAACAAAAGTGCTTTCTGCACATTTGGTTTATCCTGCA
AAGGTGACAGATCTGAAGCCAAAGGTGGAGCAGGGAAAGGTAAAAGCAGCTCCACAAAAG
CCGTTTGCTTCGAGCTACTACAGGGTGGCACCTAAACAGGAAGAGGTAGCGATTAGAAAG
GTCAATATACAAGTGCCAGCAAAGAAGGCACCAGTCAGCATAATCATCGAGTCGGATGAT
GACAGTGAAGGACGTGCGTCCTGCGTGATTTTGAAGACAGAAACAGGTAGCAAGGAGTGG
AAAGTGACAAAGCAAGGCACCGAAGAGGGCCTGGAGATCCTGCGGAGGGCGAGGAAGAGG
AGGAGGAGAGAGATGCAGTCCATCGTGCTCGCATCCTAG
SEQ ID NO: 39
Rice PAIR1, Locus# LOC_Os03g01590 Protein Sequence
MKLKMNKACDIASISVLPPRRTGGSSGASASGSVAVAVASQPRSQPLSQSQQSFSQGASA
SLLHSQSQFSQVSLDDNLLTLLPSPTRDQRFGLHDDSSKRMSSLPASSASCAREESQLQL
AKLPSNPVHRWNPSIADTRSGQVTNEDVERKFQHLASSVHKMGMVVDSVQSDVMQLNRAM
KEASLDSGSIRQKIAVLESSLQQILKGQDDLKALFGSSTKHNPDQTSVLNSLGSKLNEIS
STLATLQTQMQARQLQGDQTTVLNSNASKSNEISSTLATLQTQMQADIRQLRCDVFRVFT
KEMEGVVRAIRSVNSRPAAMQMMADQSYQVPVSNGWTQINQTPVAAGRSPMNRAPVAAGR
SRMNQLPETKVLSAHLVYPAKVTDLKPKVEQGKVKAAPQKPFASSYYRVAPKQEEVAIRK
VNIQVPAKKAPVSIIIESDDDSEGRASCVILKTETGSKEWKVTKQGTEEGLEILRRARKR
RRREMQSIVLAS*
SEQ ID NO: 40
Arabidopsis SPO11-2; Locus# AT1G63990 CDS Sequence
>AT1G63990.1

| | |
|---|---|
| 1 | ATGGAGGAAA GTTCAGGACT ATCATCGATG AAGTTCTTCT CCGATCAACA |
| 51 | CCTCTCTTAC GCTGACATTC TTCTTCCTCA CGAAGCTCGT GCTAGAATCG |
| 101 | AAGTTTCTGT TCTCAATCTC CTCCGAATTT TGAACTCCCC AGATCCAGCT |
| 151 | ATCTCCGATC TCTCTCTGAT CAATAGGAAG AGAAGCAATA GTTGTATAAA |
| 201 | CAAAGGGATA CTCACAGATG TTCATATTAT ATTCCTCTCT ACTTCGTTTA |
| 251 | CTAAGAGTTC ATTGACGAAT GCTAAAACAG CTAAAGCTTT TGTTCGTGTG |
| 301 | TGGAAAGTTA TGGAAATATG CTTTCAGATT CTGCTTCAAG AGAAACGAGT |
| 351 | CACACAAAGA GAGCTCTTCT ATAAGTTGCT TTGTGATTCA CCTGATTACT |
| 401 | TTTCATCTCA GATTGAAGTT AACAGAAGTG TCCAAGATGT GGTAGCACTT |
| 451 | CTACGTTGTA GTAGATACAG TCTTGGTATT ATGGCTTCAA GCAGAGGCCT |
| 501 | TGTTGCTGGA AGGTTATTTC TACAAGAACC AGGTAAGGAA GCTGTAGATT |
| 551 | GTTCGGCCTG TGGTTCTTCA GGTTTTGCTA TAACTGGAGA CTTGAATTTG |
| 601 | CTAGACAATA CCATCATGAG AACTGATGCT CGTTATATCA TTATTGTGGA |
| 651 | AAAGCATGCG ATCTTTCATC GGCTCGTGGA AGATCGTGTG TTCAATCACA |
| 701 | TTCCTTGCGT GTTCATCACA GCGAAAGGGT ATCCGGATAT TGCCACAAGG |
| 751 | TTTTTCCTCC ACCGGATGAG CACAACTTTT CCTGATCTGC CAATTCTTGT |
| 801 | TCTAGTTGAT TGGAATCCAG CTGGGTTAGC TATACTATGC ACCTTCAAGT |
| 851 | TTGGAAGCAT AGGGATGGGA CTTGAAGCAT ACAGATACGC TTGCAATGTG |
| 901 | AAGTGGATTG GTCTCCGAGG AGATGATCTG AATCTGATAC CAGAAGAGTC |
| 951 | TTTGGTTCCC TTAAAGCCAA AAGATTCACA GATTGCTAAG AGCTTATTGT |
| 1001 | CCTCCAAAAT ATTGCAGGAA AACTACATAG AGGAGTTGTC ACTGATGGTT |
| 1051 | CAAACTGGTA AAAGAGCGGA AATTGAAGCT CTCTATTGTC ATGGTTATAA |
| 1101 | TTATCTCGGT AAATATATAG CTACCAAGAT CGTGCAAGGC AAATACATAT |
| 1151 | AA |

SEQ ID NO: 41
Arabidopsis SPO11-2; Locus# AT1G63990 Protein Sequence

| | |
|---|---|
| 1 | MEESSGLSSM KFFSDQHLSY ADILLPHEAR ARIEVSVLNL LRILNSPDPA |
| 51 | ISDLSLINRK RSNSCINKGI LTDVSYIFLS TSFTKSSLTN AKTAKAFVRV |
| 101 | WKVMEICFQI LLQEKRVIQR ELFYKLLCDS PDYFSSQIEV NRSVQDVVAL |
| 151 | LRCSRYSLGI MASSRGLVAG RLFLQEPGKE AVDCSACGSS GFAITGDLNL |
| 201 | LDNTIMRTDA RYIIIVEKHA IFHRLVEDRV FNHIPCVFIT AKGYPDIATR |
| 251 | FFLHRMSTTF PDLPILVLVD WNPAGLAILC TFKFGSIGMG LEAYRYACNV |
| 301 | KWIGIRGDDL NLIPEESLVP LKPKDSQIAK SLLSSKILQE NYIEELSIMV |
| 351 | QTGKRAEIEA LYCHGYNYLG KYIATKIVQG KYI |

SEQ ID NO: 42
RICE OsREC8; Locus# LOC_Os05g50410 CDS Sequence
>LOC_Os05g50410.1
ATGTTCTACTCGCACCAGCTCCTCGCGCGGAAGGCTCCGCTCGGCCAGATATGGATGGCG
GCGACGCTTCACTCGAAGATCAACCGGAAGCGGCTTGACAAGCTCGACATCATCAAAATC
TGTGAGGAGATTTTGAACCCGTCGGTACCCATGGCACTAAGGCTCTCCGGAATTCTCATG
GGTGGTGTGGCGATCGTGTACGAGAGGAAGGTGAAGGCTCTGTATGATGATGTGTCTCGG
TTTCTGATTGAGATCAACGAGGCATGGCGGGTCAAGCCAGTCGCAGACCCCACCGTACTT
CCCAAGGGCAAAACCCAAGCCAAGTATGAAGCAGTAACACTGCCAGAGAATATCATGGAT
ATGGATGTGGAGCAGCCCATGCTTTTCTCAGAGGCTGATACTACAAGGTTCCGGGGAATG
CGTTTGGAGGATTTGGATGACCAATACATTAATGTCAACCTAGACGATGATGACTTCTCG
CGCGCTGAGAATCATCACCAAGCTGATGCAGAAAATATCACCCTGGCTGATAATTTCGGG
TCTGGGCTTGGAGAGACTGATGTGTTCAATCGTTTTGAGAGATTCGACATAACAGATGAT
GATGCAACTTTCAATGTCACTCCTGATGGACACCCACAGGTTCCAAGTAATCTGGTTCCT
TCTCCACCTAGGCAGGAAGACTCTCCTCAGCAACAAGAAAACCATCATGCTGCCTCATCC
CCTCTTCACGAAGAAGCTCAACAAGGGGGGGCATCTGTAAAAAATGAGCAAGAGCAGCAG
AAGATGAAGGGTCAGCAACCTGCTAAATCATCAAAGAGAAAAAAACGTAGGAAAGATGAT
GAGGTGATGATGGATAACGACCAGATAATGATCCCAGGAAATGTATATCAAACATGGCTG
AAGGATCCATCAAGCCTCATTACCAAAAGGCACAGAATCAACAGTAAAGTTAATCTTATT
CGGTCAATCAAGATAAGAGACCTCATGGACTTGCCCCTCGTTTCTCTAATATCTTCCTTG
GAGAAGTCACCCTTAGAATTTTATTATCCTAAGGAACTTATGCAGCTTTGGAAGGAATGT
ACTGAAGTCAAGTCCCCAAAAGCTCCATCTTCAGGAGGGCAGCAGTCATCATCACCAGAA
CAACAGCAAAGAAACTTGCCTCCTCAGGCATTTCCAACCCAGCCTCAGGTTGATAATGAC
AGGGAAATGGGATTTCACCCAGTGGACTTTGCAGATGACATCGAAAAACTCCGAGGAAAC
ACTAGTGGGGAATATGGAAGAGATTATGATGCTTTTCACAGTGATCATAGTGTTACTCCT
GGAAGTCCTGGGCTAAGTCGCAGGTCTGCTTCAAGCTCTGGTGGCTCTGGACGGGGATTT
ACGCAGTTGGATCCAGAAGTACAGTTGCCATCCGGAAGGTCCAAGAGGCAGCATTCATCT
GGAAAAAGCTTTGGGAACCTCGATCCAGTTGAAGAAGAATTCCCATTCGAGCAAGAACTT
AGAGATTTCAAGATGAGAAGGCTTTCAGATGTTGGGCCAACTCCAGACCTGCTGGAAGAA
ATCGAACCTACTCAAACCCCATATGAAAAGAAATCCAATCCTATCGACCAGGTCACACAA
TCAATCCACTCGTACCTCAAGCTACACTTTGACACCCCAGGGGCCTCACAGTCTGAATCA
TTAAGTCAGCTAGCACATGGGATGACTACAGCAAAGGCTGCCCGACTCTTCTATCAAGCA
TGCGTTTTAGCAACTCATGATTTTATCAAGGTTAACCAGCTGGAACCATACGGAGACATC
TTGATCTCGAGGGGACCAAAGATGTGA
SEQ ID NO: 43
RICE OsREC8; Locus# LOC_Os05g50410Protein Sequence
MFYSHQLLARKAPLGQIWMAATLHSKINRKRLDKLDIIKICEEILNPSVPMALRLSGILM
GGVAIVYERKVKALYDDVSRFLIEINEAWRVKPVADPTVLPKGKTQAKYEAVTLPENIMD
MDVEQPMLFSEADTTRFRGMRLEDLDDQYINVNLDDDDFSRAENHHQADAENITLADNFG
SGLGETDVFNRFERFDITDDDATFNVTPDGHPQVPSNLVPSPPRQEDSPQQQENHHAASS
PLHEEAQQGGASVKNEQEQQKMKGQQPAKSSKRKKRRKDDEVMMDNDQIMIPGNVYQTWL
KDPSSLITKRHRINSKVNLIRSIKIRDLMDLPLVSLISSLEKSPLEFYYPKELMQLWKEC
TEVKSPKAPSSGGQQSSSPEQQQRNLPPQAFPTQPQVDNDREMGFHPVDFADDIEKLRGN
TSGEYGRDYDAFHSDHSVTPGSPGLSRRSASSSGGSGRGFTQLDPEVQLPSGRSKRQHSS
GKSFGNLDPVEEEFPFEQELRDFKMRRLSDVGPTPDLLEEIEPTQTPYEKKSNPIDQVTQ
SIHSYLKLHFDTPGASQSESLSQLAHGMTTAKAARLFYQACVLATHDFIKVNQLEPYGDI
LISRGPKM*
SEQ ID NO: 44
Arabidopsis REC8; Locus# AT5G05490 CDS Sequence
> AT5G05490.1

| | |
|---|---|
| 1 | ATGTTGAGAC TGGAGAGTTT GATAGTAACA GTGTGGGGAC CAGCGACGCT |
| 51 | TCTAGCTCGA AAAGCTCCGT TGGGTCAGAT ATGGATGGCC GCTACATTGC |
| 101 | ACGCGAAGAT CAACCGGAAG AAACTAGATA AGCTCGATAT CATTCAAATC |
| 151 | TGCGAAGAGA TTTTGAATCC GTCGGTTCCG ATGGCTCTTA GACTCTCCGG |
| 201 | GATTCTTATG GGTGGTGTTG TGATTGTTTA TGAGAGGAAA GTGAAGCTCC |
| 251 | TATTCGATGA TGTTAATCGC TTTCTGGTTG AAATTAATGG AGCTTGGGGC |
| 301 | ACAAAATCTG TTCCGGATCC CACTTTACTA CCTAAAGGAA AAACCCATGC |
| 351 | CAGGAAAGAG GCTGTTACAT TGCCTGAGAA CGAAGAAGCT GATTTTGGAG |
| 401 | ATTTTGAACA GACTCGTAAT GTTCCTAAAT TTGGCAATTA CATGGATTTT |
| 451 | CAGCAGACTT TTATTTCCAT GCGGTTAGAT GAATCCCATG TTAACAATAA |
| 501 | CCCCGAGCCA GAAGATCTTG GACAGCAGTT CCATCAAGCT GATGCCGAGA |
| 551 | ATATCACACT CTTTGAGTAT CATGGTTCAT TCCAGACCAA CAATGAAACA |
| 601 | TATGATCGTT TTGAAAGATT TGACATCGAA GGAGATGATG AAACACAGAT |
| 651 | GAACTCCAAT CCAAGAGAAG GCGCTGAAAT ACCTACAACT CTCATCCCAT |
| 701 | CACCACCTCG TCATCATGAC ATTCCCGAAG GAGTCAACCC CACAAGCCCT |
| 751 | CAGCGCCAGG AGCAACAGGA GAATCGTAGG GACGGATTTG CTGAGCAGAT |
| 801 | GGAGGAACAA AACATACCGG ACAAAGAGGA ACACGATAGA CCACAACCAG |
| 851 | CGAAAAAGAG AGCAAGAAAG ACAGCTACTT CAGCGATGGA TTATGAGCAA |
| 901 | ACTATTATCG CTGGTCATGT TTACCAGTCA TGGCTCCAGG ATACTTCTGA |
| 951 | CATTCTCTGT AGGGGGGAAA AGAGAAAGGT TCGAGGAACT ATCCGGCCAG |
| 1001 | ACATGGAAAG TTTCAAACGT GCGAATATGC CACCTACACA ACTCTTTGAA |
| 1051 | AAGGACAGTT CTTACCCGCC TCAGCTTTAC CAGCTTTGGT CAAAGAATAC |
| 1101 | TCAAGTTCTT CAAACCTCAT CATCTGAATC TCGACATCCT GATCTCCGTG |
| 1151 | CGGAACAATC TCCAGGGTTT GTTCAGGAGA GAATGCATAA CCACCATCAA |
| 1201 | ACAGACCATC ATGAGCGCAG TGACACAAGC TCCCAAAATC TTGATAGTCC |
| 1251 | CGCAGAAATA CTCCGGACAG TTCGTACTGG GAAAGGTGCT TCAGTAGAAA |
| 1301 | GCATGATGGC TGGATCTCGA GCAAGCCCTG AAACTATTAA CCGCCAGGCT |
| 1351 | GCTGATATTA ATGTCACGCC ATTCTATTCT GGAGATGATG TGAGATCCAT |
| 1401 | GCCTAGTACA CCATCCGCAC GTGGAGCAGC TTCAATTAAC AACATAGAGA |
| 1451 | TCAGCTCTAA AAGTCGCATG CCCAATAGAA AAAGACCAAA TTCCTCACCA |
| 1501 | AGAAGAGGAC TCGAACCAGT GGCGGAAGAG AGACCGTGGG AGCACCGTGA |
| 1551 | ATATGAGTTT GAGTTTTCAA TGTTACCTGA AAAACGCTTC ACAGCCGATA |
| 1601 | AAGAAATACT ATTTGAAACT GCATCTACAC AGACTCAAAA GCCAGTGTGC |
| 1651 | AATCAATCAG ACGAGATGAT AACAGATAGC ATCAAAAGTC ACCTGAAGAC |
| 1701 | ACACTTTGAA ACACCTGGAG CTCCTCAAGT GGAATCTCTT AACAAGCTCG |
| 1751 | CTGTTGGAAT GGACAGAAAC GCTGCAGCTA AACTCTTCTT CCAATCCTGT |
| 1801 | GTGTTAGCTA CTCGCGGAGT CATCAAGGTA AACCAAGCAG AGCCTTATGG |
| 1851 | GGACATTCTC ATTGCAAGAG GACCCAACAT GTAA |

SEQ ID NO: 45
Arabidopsis REC8; Locus# AT5G05490 Protein Sequence

| | |
|---|---|
| 1 | MLRLESLIVT VWGPATLLAR KAPLGQIWMA ATLHAKINRK KLDKLDIIQI |
| 51 | CEEILNPSVP MALRLSGILM GGVVIVYERK VKLLFDDVNR FLVEINGAWR |
| 101 | TKSVPDFTLL PKGKTHARKE AVTLPENEEA DFGDFEQTRN VPKFGNYMDF |
| 151 | QQTFTSMRLD ESHVNNNPEP EDLGQQFHQA DAENITLFEY HGSFQTNNET |
| 201 | YDRFERFDIE GDDETQMNSN PREGAEIPTT LIPSPPRHHD IPEGVNPTSP |
| 251 | QRQEQQENRR DGFAEQMEEQ NIPDKEEHDR PQPAKKRARK TATSAMDYEQ |
| 301 | TIIAGHVYQS WLQDTSDILC RGEKRKVRGT IRPDMESFKR ANMPPTQLFE |
| 351 | KDSSYPPQLY QLWSKNTQVL QTSSSESRHP DLRAEQSPGF VQERMHNHHQ |
| 401 | TDHHERSDTS SQNLDSPAEI LRTVRTGKGA SVESMMAGSR ASPETINRQA |
| 451 | ADINVTPFYS GDDVRSMPST PSARGAASIN NIEISSKSRM PNRKRPNSSP |
| 501 | RPGLEPVAEE RPWEHREYEF EFSMLPEKRF TADKEILFET ASTQTQKPVC |
| 551 | NQSDEMITDS IKSHLKTHFE TPGAPQVESL NKLAVGMDRN AAAKLFFQSC |
| 601 | VLATRGVIKV NQAEPYGDIL IARGPNM |

SEQ ID NO: 46
Arabidopsis Gene Name: TAM1 (TARDY ASYNCHRONOUS MEIOSIS1); Locus
#AT1G77390 CDS Sequence
>AT1G77390.1

| | |
|---|---|
| 1 | ATGTCTTCTT CGTCGAGAAA TCTATCTCAG GAGAATCCGA TTCCTCGTCC |
| 51 | GAACTTAGCC AAGACTCGAA CCTCACTCCG CGATGTTGGA AACCGTCGTG |
| 101 | CTCCCCTCGG CGACATCACA AATCAGAAGA ATGGATCTAG AAATCCTTCA |
| 151 | CCGTCGTCTA CTCTGGTGAA TTGTTCAAAT AAGATCGGCC AATCTAAGAA |
| 201 | AGCACCAAAA CCTGCTTTAT CTCGTAATTG GAATTTGGGA ATTCTCGATT |
| 251 | CCGGTTTACC TCCCAAGCCA AATGCGAAAT CAAACATAAT CGTTCCTTAC |
| 301 | GAAGACACCG AATTGCTCCA AAGCGATGAT AGTCTTCTAT GTTCTTCACC |
| 351 | TGCATTATCC TTGGATGCCT CTCCTACTCA ATCTGACCCG TCAATTTCCA |
| 401 | CTCATGACTC TTTGACGAAC CACGTTGTAG ATTACATGGT CGAGAGCACT |
| 451 | ACTGATGATG GAAATGATGA TGATGATGAT GAGATTGTTA ACATTGATAG |
| 501 | TGACTTGATG GATCCACAGC TTTGTGCTTC TTTTGCTTGT GATATCTACG |
| 551 | AGCATTTGCG TGTATCTGAG GTGAACAAAA GACCGGCTCT AGATTACATG |
| 601 | GAAAGAACTC AGTCAAGCAT CAATGCTAGC ATGCGTTCTA TACTGATTGA |
| 651 | CTGGCTTGTG GAGGTTGCTG AAGAGTATAG GCTTTCGCCC GAGACGTTGT |
| 701 | ATTTGGCAGT AAACTACGTT GATCGGTATC TTACAGGAAA TGCAATCAAC |
| 751 | AAGCAAAATC TGCAGCTACT TGGTGTTACC TGCATGATGA TAGCAGCAAA |
| 801 | ATATGAAGAA GTGTGTGTGC CGCAAGTGGA GGATTTCTGT TACATCACTG |
| 851 | ATAACACATA CTTAAGAAAT GAGCTTTTGG AGATGGAGTC TTCTGTTCTG |
| 901 | AACTACTTGA AGTTCGAATT AACAACTCCA ACAGCAAAAT GTTTCTTGAG |
| 951 | GCGCTTTCTT CGTGCTGCTC AAGGCAGAAA GGAGGTACCA TCACTGCTGT |
| 1001 | CTGAGTGTCT GGCCTGCTAT CTCACCGAAT TATCGCTGTT AGATTACGCT |
| 1051 | ATGCTTCGAT ACGCTCCATC ACTTGTTGCA GCCTCTGCAG TTTTCTTGGC |
| 1101 | ACAATACACT CTACACCCTT CAAGAAAACC ATGGAATGCT ACGCTAGAGC |
| 1151 | ATTACACATC GTACAGGGCT AAACATATGG AAGCATGCGT TAAGAATCTT |
| 1201 | CTTCAGCTGT GTAATGAGAA ACTCTCATCT GATGTGGTTG CAATCAGAAA |
| 1251 | GAAGTACAGT CAACACAAAT ACAAGTTTGC AGCAAAGAAG CTTTGTCCCA |
| 1301 | CGTCACTACC GCAAGAGCTT TTCCTCTGA |

SEQ ID NO:47
Arabidopsis TAM1 Protein Sequence
>AT1G77390.1

| | |
|---|---|
| 1 | MSSSSRNLSQ ENPIPRFNLA KTRTSLRDVG NRRAPLGDIT MQKNGSRNPS |
| 51 | PSSTLVNCSN KIGQSKKAPK PALSRNWNLG ILDSGLPPKP NAKSNIIVPY |
| 101 | EDTELLQSDD SLLCSSPALS LDASPTQSDP SISTHDSLTN HVVDYMVEST |
| 151 | TDDGNDDDDD EIVNIDSDLM DPQLCASFAC DIYEHLRVSE VNKRPALDYM |
| 201 | ERTQSSINAS MRSILIDWLV EVAEEYRLSP ETLYLAVNYV DRYLTGNAIN |
| 251 | KQNLQLLGVT CMMIAAKYEE VCVPQVEDFC YITDNTYLRN ELLEMESSVL |
| 301 | NYLKFELTTP TAKCFLRRFL RAAQGRKEVP SLLSECLACY LTELSLLDYA |
| 351 | MLRYAPSLVA ASAVFLAQYT LHPSRKPWNA TLEHYTSYRA KHMEACVKNL |
| 401 | LQLCNEKLSS DVVAIRKKYS QHKYKFAAKK LCPTSLPQEL FL |

SEQ ID NO: 48
cyclin-A1; LOCUS# LOC_Os12g20324 CDS Sequence
>LOC_Os12g20324.3
ATGTCGACGTGCGACTCAATGAAAAGCCCAGACTTTGAGTATATTGATAATGGGGATTCC
TCCTCAGTTCTAGGTTCCTTGCAGCGAAGAGCAAACGAGAACCTGCGTATCTCAGAGGAT
AGAGATGTTGAAGAAACTAAGTGGAAGAAGGATGCTCCTTCCCCAATGGAAATCGACCAA
ATTTGTGATGTTGACAATAACTACGAGGATCCGCAGTTGTGTGCTACTCTTGCTTCTGAT
ATCTACATGCACTTGCGCGAGGCCGAGACCAGGAAACATCCATCAACCGATTTTATGGAA
ACACTCCAAAAGGATGTAAACCCAAGCATGAGAGCGATCCTGATAGACTGGCTTGTGGAA
GTCGCTGAAGAATATCGTCTTGTTCCTGATACATTATACCTGACAGTTAACTACATTGAC
CGTTATCTTTCTGGCAATGAGATCAATCGTCAAAGACTGCAATTACTTGGAGTTGCTTGT
ATGCTTATTGCTGCAAAATACAAGGAGATATGTGCACCTCAAGTAGAAGAATTCTGCTAT
ATAACTGACAACACATACTTCAGAGATGAGGTTTTGGAAATGGAAGCTTCTGTCCTGAAT
TACCTGAAGTTTGAAATGACTGCACCTACAGCAAAATGCTTTTTGAGGAGATTTGTCCGT
GTTGCACAAGTATCTGATGAGGATCCAGCATTGCATCTTGAGTTCCTAGCCAATTATGTT
GCTGAGCTATCACTGCTGGAGTACAATCTACTTTCTTACCCTCCTTCACTAGTAGCGGCA
TCAGCTATTTTCCTGGCCAAATTCATACTGCAGCCAGCAAAGCACCCTTGGAATTCCACC
CTTGCTCACTACACACAATACAAGTCGTCAGAGTTAAGCGACTGCGTTAAGGCATTGCAC
CGCCTTTTCTGTGTTGGTCCTGGGAGTAACCTTCCTGCAATCAGGGAGAAGTATACCCAA
CATAAGTACAAATTTGTGGCGAAGAAGCCCTGCCCACCCTCAATACCGACCGAATTCTTT
CGCGACTCAACATGCTGA
SEQ ID NO: 49
cyclin-A1; LOCUS# LOC_Os12g20324 Protein Sequence
MSTCDSMKSPDFEYIDNGDSSSVLGSLQRRANENLRISEDRDVEETKWKKDAPSPMEIDQ
ICDVDNNYEDPQLCATLASDIYMHLREAETRKHPSTDFMETLQKDVNPSMRAILIDWLVE
VAEEYRLVPDTLYLTVNYIDRYLSGNEINRQRLQLLGVACMLIAAKYKEICAPQVEEFCY
ITDNTYFRDEVLEMEASVLNYLKFEMTAPTAKCFLRRFVRVAQVSDEDPALHLEFLANYV
AELSLLEYNLLSYPPSLVAASAIFLAKFILQPAKHPWNSTLAHYTQYKSSELSDCVKALH
RLFCVGPGSNLPAIREKYTQHKYKFVAKKPCPPSIPTEFERDSTC
SEQ ID NO: 50
cyclin-A1; LOCUS#LOC_Os05g14730
CDS Sequence
>LOC_Os05g14730.1
ATGTCTAAGGAAGATGCTATGTCAACTGGTGATTCAACGGAAAGCCTTGATATTGATTGC
CTTGATGATGGGGACTCCGAAGTGGTATCTTCCTTGCAACATTTGGCAGATGATAAGCTT
CATATTTCTGACAACAGGGATGTTGCAGGTGTGGCATCCAAATGGACGAAGCATGGTTGT
AATTCAGTAGAAATTGATTATATCGTCGACATTGACAACAACCATGAGGATCCACAGCTG
TGTGCAACTCTTGCTTTTGACATTTACAAGCACTTGCGAGTGGCTGAGACCAAGAAAAGG
CCTTCAACAGATTTTGTGGAAACCATTCAGAAGAACATTGACACAAGCATGAGGGCAGTG
TTAATAGACTGGCTTGTGGAAGTCACAGAAGAATATCGGCTTGTACCTGAAACCTTATAC
CTCACAGTCAATTACATTGACCGGTATCTCTCGAGCAAGGTGATCAATCGGCGGAAAATG
CAATTACTTGGTGTCGCTTGCCTGCTTATAGCTTCTAAGTATGAAGAGATATGCCCACCC
CAAGTAGAAGAGCTCTGCTATATTTCTGACAATACATACACTAAGGATGAGGTTTTGAAA
ATGGAAGCTTCTGTCCTGAAATACTTGAAGTTTGAGATGACTGCACCTACAACAAAATGC
TTTTTGAGGAGATTTCTACGAGCTGCTCAAGTATGCCATGAGGCTCCAGTTTTGCATCTT
GAGTTCCTAGCTAATTACATTGCGGAGCTATCACTTCTGGAGTACAGCTTAATTTGCTAT
GTACCGTCACTTATAGCTGCGTCTTCTATTTTCTTGGCGAAGTTTATCCTTAAGCCAACA
GAGAATCCTTGGAATTCAACACTTTCATTCTACACACAATACAAACCATCCGACCTATGC
AATTGTGCAAAAGGACTACACCGGCTTTTCTTGGTTGGCCCTGGAGGCAACCTTCGAGCA
GTTAGAGAAAAATACAGTCAACACAAGTACAAATTCGTAGCAAAGAAGTACTCTCCACCA
TCAATTCCAGCAGAGTTTTTCGAAGATCCAAGCAGCTACAAGCCTGATTAA
SEQ ID NO: 51
cyclin-A1; LOCUS#LOC_Os05g14730Protein Sequence
MSKEDAMSTGDSTESLDIDCLDDGDSEVVSSLQHLADDKLHISDNRDVAGVASKWTKHGC
NSVEIDYIVDIDNNHEDPQLCATLAFDIYKHLRVAETKKRPSTDFVETIQKNIDTSMRAV
LIDWLVEVTEEYRLVPETLYLTVNYIDRYLSSKVINRRKMQLLGVACLLIASKYEEICPP
QVEELCYISDNTYTKDEVLKMEASVLKYLKFEMTAPTTKCFLRRFLRAAQVCHEAPVLHL
EFLANYIAELSLLEYSLICYVPSLIAASSIFLAKFILKPTENPWNSTLSFYTQYKPSDLC
NCAKGLHRLFLVGPGGNLRAVREKYSQHKYKFVAKKYSPPSIPAEFFEDPSSYKPD
SEQ ID NO: 52
cyclin-A1; Locus# LOC_Os01g13229 CDS Sequence
>LOC_Os01g13229.1
ATGGCCTTGGTGTGTGCGGAATGCCGTCTTGTTGACATCGTCCGGGTGCATGCGAACCTG
ATGGTACCCGAAATGGAGATCCAGCTTGGAGAAGAAGTGGGCGTCGCAAAGTTCATCAAG
CAATTCGTCGAGAACCAAAATGGGAAACATGTCCTTGGCAGTCACCGTGTTGAGGGCTCG
GTAGTCCACGTAGAAGCGCCATGGCTTGTGGACCAACAGAACCGACGACGAGAATGGCGA
CGTGTTGGGGCAGATGGAGAGCTTGTCAGCGCATTATTTCTCCAATTCGTCGTTTTGAAG
CTGTGGGTAGCGATACGGTCGAACCACTACCGGGGGCTTGCCGGGCTTGAGGTGGATGTG
ATGATTGATGGATCGGGTCGGTGGGAGGCCGGTTGGAGCGCGAAGAGGTCGTCAAATTCC
GCCAGCAAGGACGGAAGGAGATCGTCGGCGTGCAGTGCAGCGCAGCTGAGGTCGAGGGCG
AAGCAGTCGACGTCGAACACCTCGGTGGCCACCTACAGGCGAAGCCCGTGCACGACACCC
GCGCCGCGGATGCGATCGTTGTCAGCCACCACCACGCGGAGACCTTGGCGTACCGGTTGG
AGAGGTAGGCCGATGCGCTGGGCCAGCTTGGTGGTGATGAAATTGTGTGTTGAAGCCGGA
GTCAACAAGGGCCATCACCTGCTCGGCCGCGATACGCGCCACGAGGCACATCGTGCTAGT
GCCATTGACACCGAAAATCGCGTTGAGCGAGATGTGTGGTTCATCGGTGTCGTCTGTGTC
GTCCCATTCGCTGAAATTAGCCGTGGTGTCGTCGTACTCAAGAGGCCTTGTTTACAGCGC
TCTGGTAACTCTGCTGGCGAGAGACAACGAAATTGGTGTCCTGGCACGAGCCAAGGCCAT
GGCCTCCTGGAAATCCTCGGGCTGTTGGAGCTCGACATCGATGCAGATGTCGTCGGTGAG
CCCCGCTGTGAAGAGCTGAACTTGTTGGCGGTCGGTGAGGAGGTCGGACGTGCGGCTACC
CAACGCCAAAAGGCGCTGCTGGTACGTCGTCACCGTGCCAACCTGAGTGAGATGCTTGAG
TTCACCCAGGGAATTGTTGCGGATCGGCGGTCCATACTGACCGTAACAGAGCTGCTTGAA
CGTATCCCAATCCGGAGGACCCATGTGCTGCTCATAATGGAAGTACCATTCCTGTGCAGC
TCAAGTGAGATGACCAGGAAACGTCCATCAACTGATTTTATGGAAACAATCCAAAAGGAT
GTAAACCCAAGCATGAGAGCGATCCTGATAGACTGGCTTGTGGAAGTCGCTGAAGAATAT
CGTCTTGTTCCTGATACATTATACCTGACAGTTAACTACATTGATCGTTATCTTTCTGGC
AATGAGATCAATCGTCAAAGACTGCAATTACTTGGAGTTGCTTGTATGCTTATTGCTGCA
AAATACGAGGAGATATGTGCACCTCAAGTAGAAGAATTCTGCTATATAACTGACAACACA
TACTTCAGAGATGAGTGCTGGAATGAATCGAACTCTAATAACTCTCTTATTGCCTACAAC
AGGAGATTTGTCCGTGTTGCACAAGTATCGGATGAGCTTTTCATCGTGCAGGATCCAGCA
TTGCATCTTGAGTTCCTAGCCAATTATGTTGCTGAGCTATCACTGCTGGAGTACAATCTA
CTTTCTTACCCTCCTTCACTAGTAGCGGCATCGGCTATTTTCTTGGCCAAATTCATACTG
CAGCCAACAAAGCACCCTTGGAATTCCACCCTTGCTCACTACACACAATACAAGTCGTCA
GAGTTAAGCGACTGTGTAAAGGCATTGCACCGCCTTTTTAGCGTTGGTCCCGGGAGTAAC
CTTCCTGCAATCAGGGAGAAGTATACCCAACATAAGATACTGCATGCAGCTGATGTGATC
GACTTGAACATGGCAAATGCATTTAAGAATGTGAAAATATTATGTCAATGTCCCTGTCAA
TGCAACCTTCTTGAAGAAGTCATGCTCAAGCTATTTCCATACTGGAAGCTAAGCACAGCT
GTTTAG
SEQ ID NO: 53
cyclin-A1; Locus# LOC_Os01g13229 Protein Sequence
MALVCAECRLVDIVRVHANLMVPEMEIQLGEEVGVAKFIKQFVENQNGKHVLGSHRVEGS
VVHVEAPWLVDQQNRRREWRRVGADGELVSALFLQFVVLKLWVAIRSNHYRGLAGLEVDV
MIDGSGRWEAGWSAKRSSNSASKDGRRSSACSAAQLRSRAKQSTSNTSVATYRRSPCTTP
APRMRSLSATTTRRPWRTGWRGRPMRWASLVVMKLCVEAGVNKGHHLLGRDTRHEAHRAS
AIDTENRVERDVWFIGVVCVVPFAEISRGVVVLKRPCLQRSGNSAGERQRNWCPGTSQGH
GLLEILGLLELDIDADVVGEPRCEELNLLAVGEEVGRAATQRQKALLVRRHRANLSEMLE
FTQGIVADRRSILTVTELLERIPIRRTHVLLIMEVPFLCSSSEMTRKRPSTDFMETIQKD
VNPSMRAILIDWLVEVAEEYRLVPDTLYLTVNYIDRYLSGNEINRQRLQLLGVACMLIAA
KYEEICAPQVEEFCYITDNTYFRDECWNESNSNNSLIAYNRRFVRVAQVSDELFIVQDPA
LHLEFLANYVAELSLLEYNLLSYPPSLVAASAIFLAKFILQPTKHPWNSTLAHYTQYKSS
ELSDCVKALHRLFSVGPGSNLPAIREKYTQHKILHAADVIDLNMANAFKNVKILCQCPCQ
CNLLEEVMLKLFPYWKLSTAV*
SEQ ID NO: 54
cyclin-A1; Locus # LOC_Os12g31810 CDS Sequence
>LOC_Os12g31810.1
ATGGCTGGAAGGAAGGAAAATCCGGTGCTTACTGCTTGCCAAGCACCCAGTGGTCGAATC
ACACGAGCTCAAGCTGCTGCAAATCGTGGACGGTTTGGGTTTGCTCCCTCCGTATCACTA
CCCGCAAGAACTGAACGAAAGCAGACAGCAAAAGGAAAGACAAAAAGGGGAGCTTTGGAT
GAAATCACTAGTGCAAGTACTGCAACTTCAGCTCCTCAGCCTAAACGGCGCACAGTGCTC
AAGGATGTAACCAACATCGGCTGTGCCAACTCATCCAAAAATTGCACCACCACGAGCAAG
CTGCAGCAAAAGTCAAAGCCCACCCAAAGGGTGAAACAAATCCCGAGCAAAAAGCAGTGT
GCAAAGAAGGTTCCTAAGCTACCCCCTCCGGCTGTTGCTGGAACTTCATTTGTGATTGAT
TCTAAAAGTTCTGAAGAAACTCAAAAGGTGGAGCTTTTGGCAAAAGCAGAGGAACCCACA
AATTTGTTTGAAAACGAGGGGTTACTGTCATTGCAGAATATTGAGCGAAACAGGGACAGT
AATTGCCATGAGGCATTCTTTGAGGCAAGAAACGCCATGGATAAACATGAACTCGCTGAC
TCCAAGCCTGGTGACTCTAGTGGTTTAGGTTTTATAGATATTGACAATGATAATGGAAAT
CCTCAAATGTGTGCTTCCTATGCTTCAGAGATATACACAAATCTGATGGCCTCTGAGCTT
ATCAGAAGACCCAGGTCAAATTACATGGAGGCTTTGCAACGTGACATCACAAAGGGCATG
CGAGGCATTCTCATTGATTGGCTTGTTGAGGTTTCTGAAGAATATAAGCTTGTGCCAGAC
ACACTCTACCTAACCATTAATCTTATTGACCGATTTCTTTCTCAACATTATATTGAAAGA
CAGAAACTCCAACTTCTTGGAATAACAAGCATGCTGATTGCCTCGAAATATGAAGAGATA
TGTGCTCCTCGTGTTGAAGAATTTTGTTTCATAACTGACAATACATACACAAAAGCTGAG
GTGCTGAAAATGGAGGGCCTGGTGCTTAATGATATGGGGTTTCATCTATCTGTTCCAACA
ACAAAAACATTTCTCAGGAGATTCCTTAGAGCCGCACAGGCTTCTCGTAATGTTCCTTCA
ATTACCTTGGGATATCTGGCCAATTATCTTGCAGAGCTGACCCTGATCGATTACAGTTTC
CTCAAATTTCTTCCTTCAGTGGTGGCAGCATCTGCAGTCTTTCTTGCAAGATGGACACTT
GACCAATCTGACATTCCATGGAATCATACTCTTGAGCACTACACTTCTTACAAAAGCTCT
GATATTCAAATATGTGTCTGTGCTCTACGGGAACTGCAGCATAACACCAGTAATTGCCCT
CTCAATGCTATACGTGAAAAGTATAGGCAACAAAAGTTTGAGTGTGTAGCCAACCTGACA
TCACCGGAGCTGGGGCAGTCACTCTTCAGCTGA
SEQ ID NO: 55
cyclin-A1; Locus # LOC_Os12g31810 Protein Sequence
MAGRKENPVLTACQAPSGRITRAQAAANRGRFGFAPSVSLPARTERKQTAKGKTKRGALD
EITSASTATSAPQPKRRTVLKDVTNIGCANSSKNCTTTSKLQQKSKPTQRVKQIPSKKQC
AKKVPKLPPPAVAGTSFVIDSKSSEETQKVELLAKAEEPTNLFENEGLLSLQNIERNRDS
NCHEAFFEARNAMDKHELADSKPGDSSGLGFIDIDNDNGNPQMCASYASEIYTNLMASEL
IRRPRSNYMEALQRDITKGMRGILIDWLVEVSEEYKLVPDTLYLTINLIDRELSQHYIER
QKLQLLGITSMLIASKYEEICAPRVEEFCFITDNTYTKAEVLKMEGLVLNDMGFHLSVPT
TKTFLRRFLRAAQASRNVPSITLGYLANYLAELTLIDYSFLKFLPSVVAASAVFLARWTL
DQSDIPWNHTLEHYTSYKSSDIQICVCALRELQHNTSNCPLNAIREKYRQQKFECVANLT
SPELGQSLFS*
SEQ ID NO: 56
cyclin-A1; Locus # LOC_Os01g13260 CDS Sequence
>LOC_Os01g13260.1
ATGTCGAGCAACCTAGCAGCCTCCCGCCGCTCGTCGTCGTCGTCCTCGGTGGCGGCGGCG
GCGGCGGCGAAGCGACCCGCGGTGGGGGAGGGAGGAGGAGGAGGAGGAGGGAAGGCGGCA
GCGGGCGCCGCCGCGGCAAAGAAGCGCGTGGCGCTTAGCAACATCAGCAACGTCGCCGCT
GGTGGTGGCGCCCCAGGGAAGGCCGGCAATGCGAAGTTGAATTTAGCTGCCTCAGCTGCA
CCAGTGAAGAAGGGATCTTTGGCCAGTGGCCGCAATGTGGGCACGAATCGGGCCTCGGCG
GTGAAATCGGCTTCCGCCAAGCCGGCTCCGGCCATATCCCGCCATGAGAGCGCCACACAG
AAGGAGTCTGTTCTTCCTCCTAAAGTGCCTAGCATTGTGCCGACTGCTGCACTGGCACCT
GTCACTGTACCCTGCAGCAGCTTCGTCTCCCCTATGCATTCAGGAGATTCAGTTTCGGTT
GACGAGACGATGTCGACGTGTGACTCAATGAAAAGCCCAGAATTTGAGTACATTGATAAT
GGGGATTCCTCCTCAGTTCTAGGTTCCTTGCAGCGAAGAGCAAACGAAAACCTGCGTATC
TCAGAGGATAGAGATGTCGAAGAAACTAAGTGGAAGAAGGATGCTCCTTCCCCAATGGAA
ATCGACCAAATTTGTGATGTTGACAATAACTACGAGGATCCGCAGTTGTGTGCTACTCTT
GCTTCTGATATCTACATGCACTTGCGCGAGGCTGAGACCAGGAAACGTCCATCAACTGAT
TTTATGGAAACAATCCAAAAGGATGTAAACCCAAGCATGAGAGCGATCCTGATAGACTGG
CTTGTGGAAGTCGCTGAAGAATATCGTCTTGTTCCTGATACATTATACCTGACAGTTAAC
TACATTGATCGTTATCTTTCTGGCAATGAGATCAATCGTCAAAGACTGCAATTACTTGGA
GTTGCTTGTATGCTTATTGCTGCAAAATACGAGGAGATATGTGCACCTCAAGTAGAAGAA
TTCTGCTATATAACTGACAACACATACTTCAGAGATGAGGTTTTGGAAATGGAAGCTTCT
GTCCTGAATTACCTGAAGTTTGAAGTGACTGCACCTACAGCAAAATGCTTTTTGAGGAGA
TTTGTCCGTGTTGCACAAGTATCGGATGAGGATCCAGCATTGCATCTTGAGTTCCTAGCC
AATTATGTTGCTGAGCTATCACTGCTGGAGTACAATCTACTTTCTTACCCTCCTTCACTA
GTAGCGGCATCGGCTATTTTCTTGGCCAAATTCATACTGCAGCCAACAAAGCACCCTTGG
AATTCCACCCTTGCTCACTACACACAATACAAGTCGTCAGAGTTAAGCGACTGTGTAAAG
GCATTGCACCGCCTTTTTAGCGTTGGTCCCGGGAGTAACCTTCCTGCAATCAGGGAGAAG
TATACCCAACATAAGTACAAATTTGTGGCGAAGAAGCCCTGCCCACCCTCAATACCGACC
GAATTCTTTCGCGACGCAACATGCTGA
SEQ ID NO: 57
cyclin-A1; Locus # LOC_Os01g13260 Protein Sequence
MSSNLAASRRSSSSSSVAAAAAAKRPAVGEGGGGGGGKAAAGAAAAKKRVALSNISNVAA
GGGAPGKAGNAKLNLAASAAPVKKGSLASGRNVGTNRASAVKSASAKPAPAISRHESATQ
KESVLPPKVPSIVPTAALAPVTVPCSSFVSPMHSGDSVSVDETMSTCDSMKSPEFEYIDN
GDSSSVLGSLQRRANENLRISEDRDVEETKWKKDAPSPMEIDQICDVDNNYEDPQLCATL
ASDIYMHLREAETRKRPSTDFMETIQKDVNPSMRAILIDWLVEVAEEYRLVPDTLYLTVN
YIDRYLSGNEINRQRLQLLGVACMLIAAKYEEICAPQVEEFCYITDNTYFRDEVLEMEAS
VLNYLKFEVTAPTAKCFLRRFVRVAQVSDEDPALHLEFLANYVAELSLLEYNLLSYPPSL
VAASAIFLAKFILQPTKHPWNSTLAHYTQYKSSELSDCVKALHRLFSVGPGSNLPAIREK
YTQHKYKFVAKKPCPPSIPTEFFRDATC*
SEQ ID NO: 58
Cyclin-A3; Locus # LOC_Os12g39210 CDS Sequence
>LOC_Os12g39210.1
ATGGCTGACAAGGAGAACTCCACCCCGGCCTCCGCGGCGCGGCTCACCCGCTCGTCTGCG
GCGGCTGGGGCGCAGGCCAAGCGTTCGGCCGCCGCGGGCGTCGCCGACGGTGGCGCGCCG
CCGGCGAAGAGGAAGCGCGTCGCGCTCAGCGACCTCCCGACCCTCTCCAACGCCGTCGTC
GTCGCCCCCCGCCAGCCGCACCACCCCGTCGTCATCAAGCCGTCGTCCAAGCAGCCCGAG
CCCGCCGCGGAGGCGGCGGCGCCCAGCGGCGGCGGCGGCGGCTCCCCCGTGTCATCCGCG
TCGACGTCGACGGCGTCGCCCTCCTCCGGTTGGGACCCGCAGTACGCCTCCGACATCTAC
ACCTACCTCCGATCCATGGAGGTGGAGGCGCGGAGGCAGTCGGCGGCGGACTACATCGAG
GCGGTGCAGGTGGACGTGACGGCGAACATGCGGGCCATCCTCGTGGACTGGCTGGTGGAG
GTCGCCGACGAGTACAAGCTCGTCGCCGACACGCTCTACCTCGCCGTCTCCTACCTCGAC
CGCTACCTCTCCGCCCACCCGCTCAGGCGCAACAGGCTGCAGCTCCTCGGCGTCGGCGCC
ATGCTCATCGCTGCGAAGTACGAGGAGATTAGCCCTCCTCATGTGGAGGATTTCTGCTAC
ATCACTGATAATACGTACACTAGGCAGGAGGTTGTCAAGATGGAGAGCGACATACTCAAG
CTTCTCGAGTTCGAGATGGGCAATCCTACCATCAAGACATTCCTCAGGCGGTTCACGAGA
TCTTGCCAGGAAGACAAAAAGCGCTCCAGCTTGTTATTGGAGTTCATGGGGAGTTATCTT
GCTGAGCTTAGTCTACTTGACTACGGCTGTCTCCGGTTCTTGCCATCGGTGGTTGCTGCC
TCAGTGGTGTTTGTTGCTAAACTGAACATTGATCCGTACACCAATCCTTGGAGCAAGAAG
ATGCAGAAGTTGACAGGATACAAGGTGTCTGAACTGAAGGATTGCATCTTGGCCATTCAT
GACTTGCAGCTCAGAAAAAAATGTTCAAACTTAACTGCAATTCGCGACAAGTACAAGCAA
CACAAGTTCAAGTGTGTCTCAACATTGCTTCCCCCTGTTGATATCCCTGCGTCATACCTC
CAAGATTTAACAGAGTAG
SEQ ID NO: 59
Cyclin-A3; Locus # LOC_Os12g39210 Protein Sequence
MADKENSTPASAARLTRSSAAAGAQAKRSAAAGVADGGAPPAKRKRVALSDLPTLSNAVV
VAPRQPHHPVVIKPSSKQPEPAAEAAAPSGGGGGSPVSSASTSTASPSSGWDPQYASDIY
TYLRSMEVEARRQSAADYIEAVQVDVTANMRAILVDWLVEVADEYKLVADTLYLAVSYLD
RYLSAHPLRRNRLQLLGVGAMLIAAKYEEISPPHVEDFCYITDNTYTRQEVVKMESDILK
LLEFEMGNPTIKTFLRRFTRSCQEDKKRSSLLLEFMGSYLAELSLLDYGCLRFLPSVVAA
SVVFVAKLNIDPYTNPWSKKMQKLTGYKVSELKDCILAIHDLQLRKKCSNLTAIRDKYKQ
HKFKCVSTLLPPVDIPASYLQDLTE*
SEQ ID NO: 60
Cyclin-A3; Locus# LOC_Os03g41100 CDS sequence
>LOC_Os03g41100.1
ATGGCCGGCAAGGAGAACGCCGCGGCGGCGCAGCCCCGCCTCACCCGCGCCGCCGCCAAG
CGCGCGGCCGCCGTCACCGCCGTGGCCGTCGCCGCCAAGCGCAAGCGCGTCGCGCTCAGC
GAGCTCCCCACGCTGTCCAACAACAACGCCGTGGTGCTCAAGCCGCAGCCGGCGCCCAGG
GGCGGCAAGAGGGCCGCCTCCCACGCCGCCGAGCCCAAGAAGCCAGCTCCGCCGCCGGCG
CCGGCGGTGGTGGTCGTGGTCGACGACGACGAGGAGGGGGAGGGGGATCCGCAGCTCTGC
GCGCCCTACGCCTCCGACATCAACTCCTACCTCCGCTCCATGGAGGTGCAAGCGAAGCGG
CGGCCGGCGGCGGACTACATCGAGACGGTGCAGGTGGACGTGACGGCCAACATGCGAGGC
ATCCTGGTCGACTGGCTCGTCGAGGTCGCCGAGGAGTACAAGCTCGTCTCCGACACGCTC
TACCTCACCGTCTCCTACATCGACCGCTTCCTCTCCGCCAAATCCATCAACCGCCAGAAG
CTGCAGCTCCTCGGCGTCTCCGCCATGCTCATCGCCTCGAAGTATGAGGAGATCAGCCCC
CCAAATGTGGAGGATTTCTGCTATATAACCGACAATACCTATATGAAACAGGAGGTTGTC
AAGATGGAGCGCGATATACTGAATGTTCTCAAGTTTGAGATGGGCAATCCTACAACCAAG
ACGTTCCTGAGGATGTTCATCAGATCTAGCCAAGAAGACGATAAGTATCCTAGCCTTCCC
TTGGAGTTCATGTGTAGCTATCTTGCCGAGCTGAGCCTGCTGGAGTACGGCTGTGTTCGG
CTCTTGCCATCCGTTGTTGCAGCCTCAGTGGTGTTTGTTGCAAGGCTAACCCTTGATTCA
GACACCAATCCTTGGAGCAAGAAGTTGCAAGAGGTGACCGGCTACAGGGCATCTGAGTTG
AAGGATTGCATTACCTGCATACATGACTTGCAGCTAAACAGGAAAGGGTCATCTCTAATG
GCTATCCGGGACAAGTACAAGCAACATAGGTTCAAGGGCGTATCAACATTGTTACCCCCT
GTTGAGATCCCTGCATCATACTTCGAAGACCTAAACGAGTAG
SEQ ID NO: 61
Cyclin-A3; Locus# LOC_Os03g41100 Protein sequence
MAGKENAAAAQPRLTRAAAKRAAAVTAVAVAAKRKRVALSELPTLSNNNAVVLKPQPAPR
GGKRAASHAAEPKKPAPPPAPAVVVVVDDDEEGEGDPQLCAPYASDINSYLRSMEVQAKR
RPAADYIETVQVDVTANMRGILVDWLVEVAEEYKLVSDTLYLTVSYIDRFLSAKSINRQK
LQLLGVSAMLIASKYEEISPPNVEDFCYITDNTYMKQEVVKMERDILNVLKFEMGNPTTK
TFLRMFIRSSQEDDKYPSLPLEFMCSYLAELSLLEYGCVRLLPSVVAASVVFVARLTLDS
DTNPWSKKLQEVTGYRASELKDCITCIHDLQLNRKGSSLMAIRDKYKQHRFKGVSTLLPP
VEIPASYFEDLNE
SEQ ID NO: 62
Arabidopsis Gene Name: DYAD; Locus #AT5G51330, CDS Sequence

| | |
|---|---|
| 1 | ATGAGTAGTA CGATGTTCGT GAAACGGAAT CCGATTAGAG AAACCACCGC |
| 51 | CGGGAAAATC TCTTCGCCGT CGTCACCGAC TTTGAATGTT GCAGTCGCGC |
| 101 | ATATAAGAGC TGGATCTTAT TACGAAATCG ATGCTTCGAT TCTTCCTCAG |
| 151 | AGATCGCCGG AAAATCTTAA ATCGATTAGA GTCGTCATGG TGAGCAAAAT |
| 201 | CACGGCGAGT GACGTGTCTC TCCGGTACCC AAGCATGTTT TCACTCCGAT |
| 251 | CGCATTTCGA TTACAGTAGG ATGAACCGGA ATAAACCGAT GAAGAAGAGG |
| 301 | AGTGGTGGTG GTCTTCTTCC TGTTTTCGAC GAGAGTCATG TGATGGCTTC |
| 351 | GGAGCTAGCT GGAGACTTGC TTTACAGAAG AATCGCACCT CATGAACTTT |
| 401 | CTATGAATAG AAATTCCTGG GGTTTCTGGG TTTCTAGTTC TTCTCGCAGG |
| 451 | AACAAATTTC CAAGAAGGGA GGTGGTTTCT CAACCGGCGT ACAATACTCG |
| 501 | TCTCTGTCGC GCTGCTTCAC CGGAGGGAAA GTGCTCGTCT GAGCTGAAAT |
| 551 | CGGGAGGGAT GATCAAGTGG GGAAGGAGAT TGCGTGTGCA GTATCAGAGT |
| 601 | CGGCATATTG ATACTAGGAA GAATAAGGAA GGTGAGGAGA GTTCTAGAGT |
| 651 | GAAGGATGAA GTTTACAAAG AAGAAGAGAT GGAGAAAGAA GAGGATGATG |
| 701 | ATGATGGGAA TGAAATAGGA GGCACTAAAC AAGAGGCAAA GGAGATAACT |
| 751 | AATGGAAATC GTAAGAGAAA GCTGATTGAA TCAAGTACTG AGAGACTCGC |
| 801 | TCAGAAAGCT AAGGTTTATG ATCAGAAGAA GGAAACTCAA ATTGTGGTTT |
| 851 | ATAAGAGGAA ATCAGAGAGG AAGTTCATTG ATAGATGGTC TGTTGAGAGG |
| 901 | TACAAACTAG CTGAGAGGAA CATGTTAAAA GTGATGAAGG AGAAGAATGC |
| 951 | AGTGTTTGGC AACTCCATAC TCAGGCCAGA GTTGAGGTCA GAAGCAAGGA |
| 1001 | AGCTGATTGG TGACACAGGT CTATTGGATC ATCTGCTTAA GCACATGGCT |
| 1051 | GGTAAGGTGG CTCCTGGAGG TCAAGATAGG TTTATGAGAA AGCACAATGC |
| 1101 | AGATGGGGCA ATGGAGTATT GGTTGGAGAG TTCTGATTTG ATTCACATAA |
| 1151 | GGAAAGAAGC AGGAGTTAAA GATCCTTACT GGACTCCTCC ACCTGGTTGG |
| 1201 | AAGCTTGGTG ACAACCCTTC TCAAGATCCT GTCTGCGCTG GAGAAATCCG |
| 1251 | TGACATCAGA GAAGAATTAG CTAGCCTGAA AAGAGAATTG AAGAAACTTG |
| 1301 | CGTCAAAGAA GGAAGAGGAG GAGCTTGTTA TCATGACTAC GCCTAATTCT |
| 1351 | TGTGTTACTA GTCAGAATGA TAATCTGATG ACTCCAGCAA AGGAAATCTA |
| 1401 | CGCTGATCTG CTGAAAAAGA AATACAAAAT TGAGGACCAG CTAGTGATTA |
| 1451 | TTGGAGAAAC CTTGCGTAAA ATGGAGGAAG ACATGGGATG GCTTAAGAAA |
| 1501 | ACAGTGGACG AGAACTATCC TAAAAAGCCA GACTCAACAG AGACACCTTT |
| 1551 | GCTACTAGAG GATTCACCAC CAATACAGAC ACTAGAAGGA GAAGTGAAGG |
| 1601 | TGGTGAACAA GGGTAACCAA ATCACAGAGT CACCTCAAAA CAGAGAAAAA |
| 1651 | GGAAGGAAGC ATGATCAACA AGAAAGATCA CCACTTTCAC TAATAAGCAA |
| 1701 | CACTGGTTTC AGAATCTGCA GGCCTGTGGG GATGTTCGCA TGGCCCCAAT |
| 1751 | TGCCTGCTCT TGCTGCTGCT ACTGATACTA ATGCTTCTTC GCCAAGTCAC |
| 1801 | AGACAAGCCT ACCCATCCCC TTTTCCAGTC AAGCCACTTG CAGCTAAGCG |
| 1851 | TCCTCTTGGC TTGACGTTTC CCTTCACCAT CATACCCGAA GAAGCTCCCA |
| 1901 | AGAATCTCTT CAACGTTTGA |

SEQ ID NO: 63
Arabidopsis DYAD; Locus #AT5G51330 Protein Sequence

| | |
|---|---|
| 1 | MSSTMFVKRN PIRETTAGKI SSPSSPTLNV AVAHIRAGSY YEIDASILPQ |
| 51 | RSPENLKSIR VVMVSKITAS DVSLRYPSMF SLRSHFDYSR MNRNKPMKKR |
| 101 | SGGGLLPVFD ESHVMASELA GDLLYRRIAP HELSMNRNSW GFWVSSSSRR |
| 151 | NKFPRREVVS QPAYNTRLCR AASPEGKCSS ELKSGGMIKW GRRLRVQYQS |
| 201 | RHIDTRKNKE GEESSRVKDE VYKEEEMEKE EDDDDGNEIG GTKQEAKEIT |
| 251 | NGNRKRKLIE SSTERLAQKA KVYDQKKETQ IVVYKRKSER RFIDRWSVER |
| 301 | YKLAERNMLK VMKEKNAVFG NSILRPELRS EARKLIGDTG LLDHLLKHMA |
| 351 | GKVAPGGQDR FMRKHNADGA MEYWLESSDL IHIRKEAGVK DPYWTPPPGW |
| 401 | KLGDNPSQDP VCAGEIRDIR EELASLKREL KKLASKKEEE ELVIMTTPNS |
| 451 | CVTSQNDNLM TPAKEIYADL LKKKYKIEDQ LVIIGETLRK MEEDMGNLKK |
| 501 | TVDENYPKKP DSTETPLLLE DSPPIQTLEG EVKVVNKGNQ ITESPQNREK |
| 551 | GRKHDQQERS PLSLISNTGF RICRPVGMFA WPQLPALAAA TDTNASSPSH |
| 601 | RQAYPSPFPV KPLAAKRPLG LTFPFTIIPE EAPKNLFNV |

SEQ ID NO: 64
Rice homologs of AtDYAD SWITCH1; LOC_Os12g42820 CDS Sequence
>LOC_Os12g42820.1
ATGACCGCCGCCGCCGTCAACGCCTCGCGCGTCATGCGCCGCCGCGCCGCCGGCGAGGAC
CTGGGCGACGGCGACTTCTGGGCCGGTGGCGCCCCGCGCCTCTACGACTTCTCCCAGCAG
GAGCAGAAGCCGTTCTTGCCGGCGCCGCCGTCGCCCGCGCCCGTCCCGGCGTCGCCGCCG
TCCCCCGCGGCGGAGTCGGTGGCGCCGTGCCTCCTCACGCTGCAGTGCAGCGGCGTCGGG
TGGGGGGTCAGGAAGCGGGTCAGGTACGTCGGGAGGCACCACCACCTCGCGCGCCACCAC
GCTCCCGAACGCGCCGTGGACGCCGCGCGGGACGACGACGAGGCGAGCTCCGCCAAGGCC
AAGAACGAGAGCCCGAAGGAGGAGGCGGCGGCGGCAGAAGAAGACGACGACGTCGAACAC
AAGGTGGCGGTGCGCACCACGTCGGAGGAGAAGAAGAAGAAGAGGAGGAGGAAGCGCGGC
CGTGGCCGTGTCCGTGGCCATGGCGTCGCCAAGCGTCCCAAGAAGGAGGATGAGGAGGGG
ACGAAGCTCTCGGCTCCCAAGGCCGAGCAGCTCGAGGAGGAGGAGGAGGGCGCCGCGGTG
GCGGCGCCGAGCGGCATGATCGACCGGTGGAAGGCGACCCGGTACGCCACCGCGGAGGCG
TCGCTGCTCGCCATCATGCGCGCCCACGGCGCGCGCGCCGGGAAGCCCGTCCCGCGCGCG
GCGCTGCGGGAGGAGGCGCGCGCCCACATCGGCGACACGGGCCTCCTCGACCACCTCCTC
AGGCACATCGCCGACAAGGTGGCGCCCGGCGGCGCCGAGCGGTTCCGGCGGCGGCACAAC
GCCGGCGGCGGGCTGGAGTACTGGCTGGAGCCCGCCGAGCTCGCCGCCGTGCGGCGGAAG
GCCGGCGTGGCCGACCCGTACTGGGTGCCTCCTCCCGGATGGAAGCCAGGGGACCCCGTG
TCGCCGGAGGGCTACTTGCTGGAGGTGAGGAAGCAGGTGGAGCAGCTCGCCGTTGAGCTC
GCCGGCGTCAGAAGGCACATGGATCACCTCACTTCCAATGTGAGTCAAGTGGGCAAGGAA
ATCAAATCTGAGGCTGAGAAGTCCTACAATACATGTCAGGGTGGGGACCCACCCTACCTT
GACCGGATCTCGATCCGTGCCTTCGCCCGGAAGCTAAGCCGCAGCTCGTCCTTGAGCGGG
ACCCAGCGCCGTCCCCCGCCTGACACGGGGGACAGCCCTGTCATTCCCTCATAA
SEQ ID NO: 65
SWITCH1; LOC_Os12g42820 Protein Sequence
MTAAAVNASRVMRRRAAGEDLGDGDFWAGGAPRLYDFSQQEQKPFLPAPPSPAPVPASPP
SPAAESVAPCLLTLQCSGVGWGVRKRVRYVGRHHHLARHHAPERAVDAARDDDEASSAKA
KNESPKEEAAAAEEDDDVEHKVAVRTTSEEKKKKRRRKRGRGRVRGHGVAKRPKKEDEEG
TKLSAPKAEQLEEEEEGAAVAAPSGMIDRWKATRYATAEASLLAIMRAHGARAGKPVPRA
ALREEARAHIGDTGLLDHLLRHIADKVAPGGAERFRRRHNAGGGLEYWLEPAELAAVRRK
AGVADPYWVPPPGWKPGDPVSPEGYLLEVRKQVEQLAVELAGVRRHMDHLTSNVSQVGKE
IKSEAEKSYNTCQGGDPPYLDRISIRAFARKLSRSSSLSGTQRRPPPDTGDSPVIPS*
SEQ ID NO: 66
Locus #LOC_Os12g42830 CDS Sequence
>LOC_Os12g42830.1
ATGACCGCCGCCGCCGTCAACGCCTCGCGCGTCATGCGCCGCCGCGCCGCAGGCGAGGAC
CTGGGCGACGGCGGCGATGGCGACGGCGACTTCTGGGCCGGTGGCGCCCCCCGCCTCTAC
GACTTCTCCCAGCAGGAGCAGAAGCCGTTCTTGCCCGCGCCCGCGCCCGCGCCGCCGTCG
CCCGCGCCCGTCCCGGCGTCGCCGCCGTCCCCCGCGGCGGAGTCGGTGGCGCCGTGCCTC
CTCACGCTGCAGTGCAGCGGCGTCGGGTGGGGTGTCAGGAAGCGGGTCCGGTACGTCGGG
AGGCACCACCACCTCGCGCGCCACCACGCTCCCGAGCGCGCCGTGGACGCCGCGCGGGAC
GACGACGAGGCGAGCTCCGCCAAGGCCAAGAACGAGAGCCCGAAGGAGGAGGCAGCGGCG
GCAGAAGAAGACGACGACAACGTCGAACACAAGGTGGCGGTGCCCACCACGTCGGAGGAG
AAGAAGAGGAGGAGGAGGAGGAAGCGTGGCCGTGGCCGTGTCGGTGGCCATGGCGTCGCC
AAGCGTCCCAAGAAGGAGGAGGAGGAGGAGGAGACGAAGCTCTCGGCTCCCAAGGCCGAG
CAGCTCGAGGAGGAGGAGGGCGCCGCGGTGGCGGCGCCGAGCGGCATGATCGACCGGTGG
AAGGCGACCCGGTACGCCACCGCGGAGGCGTCGCTGCTCGCCATCATGCGCGCCCGCGGC
GCGCGCGCCGGGAAGCCCGTCCCGCGCGGGGCGCTGCGGGAGGAGGCGCGCGCCCACATT
GGCGACACGGGCCTCCTCGACCACCTCCTCAGGCACATCGCCGACAAGGTGGCGCCCGGC
GGCGCCGAGCGGTTCCGGCGGCGGCACAACGCCGGCGGCGGGCTGGAGTACTGGCTCGAG
CCCGCCGAGCTCGCCGCCGTGCGGCGGAACGCCGGCGTGGCCGACCCGTACTGGGTGCCT
CCTCCCGGATGGAAGCCAGGGGACCCCGTCTCGCCGGAGGGCTACTTGCTGGAGGTGAGG
AAGCAGGTGGAGAAGCTCGCCGTGGAGCTCGCCGGCGTCAGAAGGCACATGGATCACCTC
TCTTCCAATGTGAGTCAAGTGGGCAAGGAAATCAAATCTGAGGCTGAGAAATCCTACAAT
ACATGCCAGGAGAAGTATGCCTGTATGGAGAAAGCCAATGGCAATCTGGAAAAGCAGCTT
CTGTCCTTGGAGGAGAAGTATGAGAATGCAACACACGCAAATGGCGAGCTGAAGGAGGAG
TTGTTGTTTCTCAAGGAGAAGTTTGTGAGTGTGGTCGAGAACAACACCAGACTGGAGCAC
CAGCTGACTGCTTTATCCACTTCTTTCCTGTCTCTAAAGGAGGAACTGCTCTGGCTGGAA
AAAGAAGAAGCTGATCTGTATGTCAAGGAACCATGGGAAGACGACGATGAAAAGCAAGAA
CACGATGCCGGGAAAGAGGCGAAGGACGACGATGTCGCCGGCGTCAGTGCAGCCAACGAC
CAGCCGGACGTCGACGGCGATGGCACCACCACCACCACCACCACCAGCAGCAATGGTGGC
AGCGGGAAGAGAACATCGAGGAAGTGCAGCGTGCGCATCTCCAAGCCGCAGGGCGCGTTC
CAGTGGCCGACGCCGAGCCTGCCGTTCTCGCCGGAGCTCGCCGCGCCGCCGTCGCCGCCG
CTGACCCCGACGGCGCCCGTCGTCGCCGGCGCCGCCAACTTCGCCACCATGGACGAGCTC
TACGAGTACATGATGGCCGGCGGCCTCCCCACGCCACCGTCCACCACCAGCAACGCCGGG
AAGCTCCCCTCGCTGCCCGCCGCCACGGCCTGCGCCACGACGCCGCCGGTGAAGACGGCG
GACGCCGCCGGCGACGTGGGCACCGAGCTGGCACTGGCCACTCCCGCCTACTGA
SEQ ID NO: 67
Locus #LOC_Os12g42830 Protein sequence
MTAAAVNASRVMRRRAAGEDLGDGGDGDGDFWAGGAPRLYDFSQQEQKPFLPAPAPAPPS
PAPVPASPPSPAAESVAPCLLTLQCSGVGWGVRKRVRYVGRHHHLARHHAPERAVDAARD
DDEASSAKAKNESPKEEAAAAEEDDDNVEHKVAVPTTSEEKKRRRRRKRGRGRVGGHGVA
KRPKKEEEEEETKLSAPKAEQLEEEEGAAVAAPSGMIDRWKATRYATAEASLLAIMRARG
ARAGKPVPRGALREEARAHIGDTGLLDHLLRHIADKVAPGGAERFRRRHNAGGGLEYWLE
PAELAAVRRNAGVADPYWVPPPGWKPGDPVSPEGYLLEVRKQVEKLAVELAGVRRHMDHL
SSNVSQVGKEIKSEAEKSYNTCQEKYACMEKANGNLEKQLLSLEEKYENATHANGELKEE
LLFLKEKFVSVVENNTRLEHQLTALSTSFLSLKEELLWLEKEEADLYVKEPWEDDDEKQE
HDAGKEAKDDDVAGVSAANDQPDVDGDGTTTTTTTSSNGGSGKRTSRKCSVRISKPQGAF
QWPTPSLPFSPELAAPPSPPLTPTAPVVAGAANFATMDELYEYMMAGGLPTPPSTTSNAG
KLPSLPAATACATTPPVKTADAAGDVGTELALATPAY*
SEQ ID NO: 68
SWI1; LOCUS# LOC_Os03g44760 CDS Sequence
>LOC_Os03g44760.1
ATGGACGCGGAGATGGCGGCTCCTGCGCTTGCGGCAGCTCATCTGCTGGACTCGCCCATG
AGGCCACAGGTGAGCAGATACTACTCCAAGAAGAGGGGTAGCAGCCACAGCAGAAATGGC
AAGGATGATGCCAACCATGACGAGTCCAAGAACCAATCACCCGGCTTGCCCCTGAGCAGA
CAGAGCCTGTCCTCATCTGCCACCCACACCTACCACACCGGAGGGTTCTACGAGATCGAC
CACGAGAAGCTTCCCCCCAAATCCCCAATTCATCTCAAGTCCATACGCGTGGTAAAGGTG
AGCGGCTACACAAGCCTGGACGTCACAGTGAGCTTCCCGTCCCTCCTGGCGCTGCGAAGC
TTCTTCTCCTCCTCCCCACGGTCGTGCACTGGGCCGGAGCTCGACGAGCGCTTCGTCATG
AGCAGCAACCACGCGGCCCGCATCCTGCGCCGTCGGGTGGCCGAGGAGGAGCTCGCGGGC
GACGTGATGCACCAGGACAGCTTCTGGCTCGTCAAGCCCTGCCTCTATGACTTCTCCGCG
TCGTCACCACATGATGTGCTGACCCCGTCGCCGCCGCCTGCCACAGCGCAGGCGAAGGCG
CCGGCAGCCAGTTCCTGCCTTCTCGACACCTTGAAGTGCGACGGCGCCGGGTGGGGCGTG
AGGCGCCGTGTCAGGTACATTGGTCGCCACCACGATGCTTCCAAGGAGGCCAGCGCTGCC
AGCCTCGATGGCTACAACACAGAGGTCAGCGTCCAGGAGGAGCAGCAGCAGCGACTGCGG
CTTCGACTGCGGTTGCGACAACGCCGGGAGCAGGAAGACAACAAGAGCACTAGCAATGGC
AAGAGGAAGCGGGAGGAGGCAGAGAGCAGCATGGACAAGAGCAGAGCCGCCAGGAAGAAG
AAAGCCAAGACTTACAAGAGTCCCAAGAAGGTGGAGAAGAGGCGCGTCGTGGAGGCTAAA
GACGGCGACCCTCGGCGCGGCAAGGACCGGTGGTCGGCCGAGCGGTACGCAGCGGCGGAG
AGGAGCCTGCTGGATATAATGCGCTCCCATGGTGCCTGCTTCGGTGCGCCGGTGATGCGG
CAGGCTCTGCGGGAGGAAGCCCGCAAGCATATCGGTGACACCGGCCTCCTTGACCACCTG
CTCAAGCACATGGCCGGCAGGGTACCGGAAGGCAGCGCGGACCGGTTCCGTCGCCGGCAC
AATGCGGATGGTGCCATGGAGTACTGGCTGGAGCCGGCGGAGCTTGCCGAGGTACGGCGG
CTGGCTGGAGTGTCTGATCCATACTGGGTGCCGCCACCTGGGTGGAAGCCAGGTGATGAC
GTGTCCGCAGTCGCCGGTGACCTCCTGGTCAAGAAGAAGGTGGAAGAGCTCGCTGAGGAG
GTTGATGGTGTAAAAAGGCACATCGAGCAGCTCAGTTCTAATTTGGTGCAGCTGGAGAAG
GAAACAAAATCTGAGGCAGAGCGATCTTACAGCTCTAGGAAGGAGAAGTATCAGAAGTTG
ATGAAGGCAAATGAAAAGCTCGAGAAACAGGTGTTATCTATGAAGGACATGTATGAGCAT
CTGGTTCAGAAAAAGGGTAAGCTGAAGAAGGAGGTGCTGTCCTTGAAGGATAAATACAAG
CTTGTGCTGGAGAAGAATGATAAACTGGAGGAACAGATGGCTAGTCTCTCCAGCTCCTTC
CTTTCTTTGAAGGAACAATTGCTGCTGCCAAGAAATGGAGATAATCTGAACATGGAAAGG
GAAAGGGTGGAAGTGACTTTGGGCAAGCAAGAAGGCCTTGTTCCCGGCGAACCACTGTAT
GTTGATGGTGGTGACCGGATCAGCCAGCAAGCAGATGCCACCGTCGTCCAAGTCGGCGAG
AAGAGGACGGCGAGGAAGAGCAGCTTCCGCATCTGCAAGCCACAGGGAACGTTCATGTGG
CCACACATGGCGTCTGGCACGAGCATGGCCATCAGTGGGGGAGGCAGCAGCAGCTGCCCT
GTCGCCTCCGGGCCAGAGCAGCTCCCTCGCAGCAGCAGCTGCCCCAGCATTGGGCCTGGT
GGCCTCCCGCCGTCGTCACGAGCCCCAGCCGAGGTGGTGGTCGCGTCGCCACTGGACGAG
CACGTGGCGTTCCGCGGGGGCTTCAACACGCCGCCCTCGGCATCGTCCACCAACGCCGCC
GCTGCCGCCAAGCTGCCTCCCCTGCCCAGCCCGACGTCACCTCTCCAGACACGGGCCCTG
TTCGCCGCTGGCTTCACTGTCCCGGCATTACACAACTTCTCCGGCCTCACCTTACGCCAT
GTGGACTCCTCGTCGCCGTCGTCCGCGCCATGCGGTGCTAGGGAGAAGATGGTGACCCTG
TTCGATGGAGACTGCCGGGGGATCAGCGTCGTGGGCACCGAGCTGGCACTGGCCACTCCG
TCCTACTGCTGA
SEQ ID NO: 69
SWI1; LOCUS# LOC_Os03g44760 Protein sequence
MDAEMAAPALAAAHLLDSPMRPQVSRYYSKKRGSSHSRNGKDDANHDESKNQSPGLPLSR
QSLSSSATHTYHTGGFYEIDHEKLPPKSPIHLKSIRVVKVSGYTSLDVTVSFPSLLALRS
FFSSSPRSCTGPELDERFVMSSNHAARILRRRVAEEELAGDVMHQDSFWLVKPCLYDESA
SSPHDVLTPSPPPATAQAKAPAASSCLLDTLKCDGAGWGVRRRVRYIGRHHDASKEASAA
SLDGYNTEVSVQEEQQQRLRLRLRLRQRREQEDNKSTSNGKRKREEAESSMDKSRAARKK
KAKTYKSPKKVEKRRVVEAKDGDPRRGKDRWSAERYAAAERSLLDIMRSHGACFGAPVMR
QALREEARKHIGDTGLLDHLLKHMAGRVPEGSADRFRRRHNADGAMEYWLEPAELAEVRR
LAGVSDPYWVPPPGWKPGDDVSAVAGDLLVKKKVEELAEEVDGVKRHIEQLSSNLVQLEK
ETKSEAERSYSSRKEKYQKLMKANEKLEKQVLSMKDMYEHLVQKKGKLKKEVLSLKDKYK
LVLEKNDKLEEQMASLSSSFLSLKEQLLLPRNGDNLNMERERVEVTLGKQEGLVPGEPLY
VDGGDRISQQADATVVQVGEKRTARKSSFRICKPQGTFMWPHMASGTSMAISGGGSSSCP
VASGPEQLPRSSSCPSIGPGGLPPSSRAPAEVVVASPLDEHVAFRGGFNTPPSASSTNAA
AAAKLPPLPSPTSPLQTRALFAAGFTVPALHNFSGLTLRHVDSSSPSSAPCGAREKMVTL
FDGDCRGISVVGTELALATPSYC
SEQ ID NO: 70
ECS1 PROMOTER. Sense strand:
gagatttgggaaatgtgcaatttgggtttatctggttttgttgttttggttatttagttttgagtccggtttgaagaaatgcttcatagatatataa
atgtaaccagaaatataaataaatcataactaagtagtattatatttttgttaagcttaattatttcaattccaagtctttcctaagaatttgttgaa
aatttataatttacgttacactttgtaaaatcagaacgatccaattcacaagattaatgctacgactctgtttttttctttaaaaataaatcaataa
tcatctccactaaacctctcaataacttagcagtcttaatgaaatttaaagctaatctatcaatcacattctcaccacgtcgccaaactcgttg
ccgtttcaatcttttcaagttccctttcatgctgtttataaacccttgcactctttcactcacagactcactacaagtctacaccacaaacttac
caaatcatccaaaa
SEQ ID NO: 71
ECS2 PROMOTER. Sense strand:
acacgttatggaaagcaaagaataacaaaagtaatattcttacctatcttttttagttggaaaacttgcattgtgtaacgtattcaaacattttc
gaaatggtttattggtttttgtatataattaatttgggttaaactgatatattttatgagataaatatagaatctcatgtgcttatacaaagcaact
atattaattttgttaaccgtaagttacaaaaacagtggtcggaggaaatcaggaaaataaaaagagagaaaagagtctacacaatgggc
caattattataagtaaatgatagtcatgaaagcccatttcagaagaagatcttttggaaatgagaatagtgctctaggctcactggtccttttt
actattggtatagaaactgtcaaagcccaacaggtttaaactagcatttcaggcgctgtaattcttctgcagtttgtttgtataaacttggaat
atggatgggttaaacactgatatttcttcactcgttttgtctcacatactgttcgatgcttaacacaggtcttaaAAAGAAACTGG
GTTTGATGTCTCAAATCTACTCAAGAAAGAAAGATATCTTGAGTTTGCATCGAGA
CAGAAAAGAGTACGACTATACATAGCTGCTGGGGTAGTAGCCTGCAGAGAATAC
AGATTTTGAACCACGTACAAGGAACCAAATCAGTGTATGTATCTAACTATTAACC
TTGTGGTGTGATCTTGTCCTCTTAGGTATTGTGGAATCCTTGTAGGAAATGTCATG
GCAACTCATTAGTCATCTTGAACCAAATGAGATGATACATGATGGTCTCAAATTG
GACATGGTGGCACCTTTTGTTTCGTGAGTGGCTTTCAATTTATCTCCATAGAAATT
GTTTAATTTTCGTTATTGGTGCCTGTCAATAAAAATTACAAACATATGCAGAGCG
TTGGATTCGTGGATCGTTGAACATCCTATTGAGAGACAGGGCCAGCCTCCTAATT
GTATGACATCGTCTCTTTCACAATATACTCATTAAATGAGAGGTTGAGATTTGAC
TTATTTGCTTTATACAGCCTGCACAGTGTGGAAGACCCCTCTAAAGACTGAACTG
GGGACAGCAACAATGGGAATCTGAccatcctcatgacagtacctggaaagagtctcagaagcttcaagttcagt
acgcagcttgaccagtctttcagtcatatagccataggggttgaattagtgtccatcttcccattgtgattaacgttctgatttagcatgcac
cttcgaattaagtgaatctattaccatgtgaccaagccattgcattactaatataagcatatcacatttcccttttctccgtgccaactgaattt
gaattattttccctcaacttaatcacatgttttcctcacggccaaaagtactctcagtgttacatgattaccacaacaaatgatttaaactttga
acttttgaagttatcgagcaacatggcaaatcctggtcctatatgacataacatgagttcctctgcctattgtaaaattaggaaacacaaaa
ccaaaatgattatatctggtattatagtgtggtgtataacatatactcacacaagatatgctcttaagatgataaatgtctaatcttccaagtc
ccaattttgaaaacgttgatattaatttcccctcaaccccactagcctcaaattaaattagcagccttagtgtgaaattaaaagatagctaat
gaattgcatttcagactttcacctccccactcacgtagctataactccttaccgtttcaaatctcttcacttccccaattttgttgtgtataaaaa
cctcttctccacttcactctttccaccacaaactttctaaaactaatcaaca
SEQ ID NO: 72:
Amborella trichopoda DWT1 protein sequence
MASSNRHWPSMFKSKPCNQWQHDINSPLICQKPPFTAEERSPEPKPRWNPKPEQIRILEAIFNSGMVN
PP
REEIRRIRAQLQEYGQVGDANVFYWFQNRKSRSKHKHKQLHQSSAKPATPSPPTVPNQNYQPTPQSSQ
TP
NSSSSSSEKSEASPVQLGSIKPGATVNVMEGLNAANSPTCSVNQVAYLGSQPEPSPLFFQTESGCEMS
AF
SELANMLQQQEKMKMGHIAMNDILNGVGEGTANSNGCSGGGGRVTVFINEMAFEVGAGGRVNVREAFG
EA
MLIHSSGHPVPTNEWGFTLQPLQHGHFYYLV
SEQ ID NO: 73:
Amborella DWT1 protein conserved 67 amino acid domain
PEPKPRWNPKPEQIRILEAIFNSGMVNPPREEIRRIRAQLQEYGQVGDANVFYWFQNR
KSRSKHKHK

1. A plant comprising:
a first expression cassette comprising a first plant egg-specific promoter operably linked to a polynucleotide encoding a Dwarf Tiller 1 (DWT1) polypeptide; and
a second expression cassette comprising a second plant egg-specific promoter operably linked to polynucleotide encoding a Babyboom polypeptide,
wherein the plant has more efficient parthenogenesis than a control plant lacking at least the first, and optionally the second, expression cassette.

2. The plant of claim 1, wherein the plant is diploid and progeny from the plant resulting from parthenogenesis are haploid.

3. The plant of claim 1, wherein the plant further comprises sufficient mitosis instead of meiosis (MiME) expression cassettes comprising a promoter operably linked to gRNAs to induce a MiME phenotype such that the plant produces clonal seed.

4. The plant of claim 3, wherein the MiMe expression cassettes comprise:
an expression cassette comprising a promoter operably linked to a gRNA that targets OSD1 or an ortholog thereof;
an expression cassette comprising a promoter operably linked to a gRNA that targets ATREC8 or an ortholog thereof;
an expression cassette comprising a promoter operably linked to a gRNA that targets SPO11, or PRD1, or PRD2 or PRD3/PAIR1 or an ortholog thereof.

5. The plant of claim 1, wherein the DWT1 polypeptide comprises an amino acid sequence at least 80%, 85%, 90%, 95%, or 98% identical to SEQ ID NO:33.

6. The plant of claim 1, wherein the DWT1 polypeptide comprises an amino acid sequence at least 65%, 70%, 75%, 80%, 85%, 90%, 95%, or 98% identical to SEQ ID NO: 1, 2, 3, 4, 5, 6 or 72.

7. The plant of claim 1, wherein the Babyboom polypeptide comprises an amino acid sequence at least 80%, 85%, 90%, 95%, or 98% identical to SEQ ID NO: 10-29.

8. The plant of claim 1, wherein the first egg-specific promoter and the second egg-specific promoter are the same.

9. The plant of claim 1, wherein the first egg-specific promoter and the second egg-specific promoter are the different.

10. The plant of claim 1, wherein the first egg-specific promoter and the second egg-specific promoter or both comprise SEQ ID NO:30, SEQ ID NO:31 or SEQ ID NO:32.

11. The plant of claim 1, wherein the plant is a rice plant.

12. A method of making the plant of claim 1, the method comprising,
introducing the first expression cassette and the second expression cassette into the plant.

13. The method of claim 12, wherein the introducing comprises transformation of the plant with the first or second or both expression cassettes, introducing the first or second or both expression cassettes into the plant with a sexual cross, or introducing one of the first and second expression cassettes into the plant via transformation and introducing one of the first and second expression cassettes into the plant via a sexual cross.

14. A method of generating haploid progeny, the method comprising
cultivating a plant of claim 1; and
collecting haploid seed from the plant.

15. A method of generating clonal progeny, the method comprising
growing a plant of claim 1, and
collecting clonal seed from the plant.

16. A nucleic acid comprising an expression cassette comprising a plant egg-specific promoter operably linked to a polynucleotide encoding a DWT1 polypeptide.

17. The nucleic acid of claim 16, wherein the promoter comprises SEQ ID NO: 30, SEQ ID NO:31 or SEQ ID NO:32.

18. The nucleic acid of claim 16, wherein the DWT1 polypeptide comprises an amino acid sequence at least 80%, 85%, 90%, 95%, or 98% identical to SEQ ID NO:33.

19. The nucleic acid of claim 16, wherein the DWT1 polypeptide comprises an amino acid sequence at least 65%, 70%, 75%, 80%, 85%, 90%, 95%, or 98% identical to SEQ ID NO: 1, 2, 3, 4, 5, 6 or 72.
